(19) United States
(12) Patent Application Publication
(10) Pub. No.: US 2008/0306111 A1
(43) Pub. Date: Dec. 11, 2008

(54) TRICYCLIC DELTA- OPIOID MODULATORS

(76) Inventors: John R. Carson, Norristown, PA (US); Scott L. Dax, Landenberg, PA (US); Bart DeCorte, Southhampton, PA (US); Li Liu, Doylestown, PA (US); Mark McDonnell, Lansdale, PA (US); James McNally, Souderton, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/187,680

(22) Filed: Aug. 7, 2008

Related U.S. Application Data
(63) Continuation of application No. 11/314,300, filed on Dec. 21, 2005.
(60) Provisional application No. 60/638,314, filed on Dec. 22, 2004.

(51) Int. Cl. A61K 31/4545; C07D 405/14; A61K 31/4535; C07D 409/04; A61K 31/453

(52) U.S. Cl. 514/318; 546/196; 514/320; 546/194; 546/202; 514/324

(57) ABSTRACT

The invention is directed to delta opioid receptor modulators. More specifically, the invention relates to tricyclic δ-opioid modulators. Pharmaceutical and veterinary compositions and methods of treating mild to severe pain and various diseases using compounds of the invention are also described.

### CROSS-REFERENCE TO RELATED APPLICATIONS

This is a continuation of application Ser. No. 11/314,300, filed Dec. 21, 2005, which claims the benefit of U.S. application Ser. No. 60/638,314, filed Dec. 22, 2004, which is incorporated herein in its entirety.

### STATEMENT REGARDING FEDERALLY SPONSORED RESEARCH OR DEVELOPMENT

The research and development of the invention described below was not federally sponsored.

### BACKGROUND OF THE INVENTION

The term “opiate” has been used to designate pharmacologically active alkaloids derived from opium, e.g., morphine, codeine, and many semi-synthetic congeners of morphine. After the isolation of peptide compounds with morphine-like actions, the term opioid was introduced to refer generically to all drugs with morphine-like actions. Included among opioids are various peptides that exhibit morphine-like activity, such as endorphins, enkephalins and dynorphins. However, some sources use the term “opiate” in a generic sense, and in such contexts, opiate and opioid are interchangeable. Additionally, the term opioid has been used to refer to antagonists of morphine-like drugs as well as to characterize receptors or binding sites that combine with such agents.

Opioids are generally employed as analgesics, but they may have many other pharmacological effects as well. Morphine and related opioids produce certain of their major effects on the central nervous and digestive systems. The effects are diverse, including analgesia, drowsiness, mood changes, respiratory depression, dizziness, mental clouding, dysphoria, pruritus, increased pressure in the biliary tract, decreased gastrointestinal motility, nausea, vomiting, and alterations of the endocrine and autonomic nervous systems.

When therapeutic doses of morphine are given to patients with pain, they report that the pain is less intense, less discomforting, or entirely gone. In addition to experiencing relief of distress, some patients experience euphoria. However, when morphine in a selected pain-relieving dose is given to a pain-free individual, the experience is not always pleasant; nausea is common, and vomiting may also occur. Drowsiness, inability to concentrate, difficulty in mentation, apathy, lessened physical activity, reduced visual acuity, and lethargy may ensue.

Two distinct classes of opioid molecules can bind opioid receptors: the opioid peptides (e.g., the enkephalins, dynorphins, and endorphins) and the alkaloid opiates (e.g., morphine, etorphine, diprenorphine and naloxone). Subsequent to the initial demonstration of opiate binding sites (Pert, C. B. and Snyder, S. H., Science (1973) 179:1011-1014), the differential pharmacological and physiological effects of both opioid peptide analogues and alkaloid opiates served to delineate multiple opioid receptors. Accordingly, three molecularly and pharmacologically distinct opioid receptor types have been described: delta, kappa and mu. Furthermore, each type is believed to have sub-types (Wollemann, M., J Neurochem (1990) 54:1095-1101; Lord, J. A., et al., Nature (1977) 267:495-499).

All three of these opioid receptor types appear to share the same functional mechanisms at a cellular level. For example, the opioid receptors cause inhibition of adenylate cyclase, and inhibition of neurotransmitter release via both potassium channel activation and inhibition of Ca²⁺ channels (Evans, C. J., In: Biological Basis of Substance Abuse, S. G. Korenman & J. D. Barchas, eds., Oxford University Press (in press); North, A. R., et al., Proc Natl Acad Sci USA (1990) 87:7025-29; Gross, R. A., et al., Proc Natl Acad Sci USA (1990) 87:7025-29; Sharma, S. K., et al., Proc Natl Acad Sci USA (1975) 72:3092-96). Although the functional mechanisms are the same, the behavioral manifestations of receptor-selective drugs differ greatly (Gilbert, P. E. & Martin, W. R., J Pharmacol Exp Ther (1976) 198:66-82). Such differences may be attributable in part to the anatomical location of the different receptors.

Delta receptors have a more discrete distribution within the mammalian CNS than either mu or kappa receptors, with high concentrations in the amygdaloid complex, striatum, substantia nigra, olfactory bulb, olfactory tubercles, hippocampal formation, and the cerebral cortex (Mansour, A., et al., Trends in Neurosci (1988) 11:308-14). The rat cerebellum is remarkably devoid of opioid receptors including delta opioid receptors.

D. Delorme, E. Roberts and Z. Wei, World Patent WO/28275 (1998) discloses diaryl methylidenylpiperidines that are opioid analgesics, but does not disclose or suggest the compounds of the present invention.

C. Kaiser, and others (J. Med. Chem. 1974, Volume 17, pages 57-61) disclose some piperidylidene derivatives of thioxanthenes, xanthenes, dibenoxepins and acridans that are neuroleptic agents. These authors, however, do not disclose or suggest either the structure or the activity of the compounds of the present invention.

British Patent GB 1128734 (1966) discloses derivatives of 6,11-dihydrodibenzo[b,e]oxepine that are anticholinergic, anti-convulsive, muscle-relaxing, sedating, diuretic, and/or vasoactive agents. These, agents, however, differ significantly from the compounds of the present invention both structurally and pharmacologically.

There is a continuing need for new delta opioid receptor modulators as analgesics. There is a further need for delta opioid receptor selective agonists as analgesics having reduced side effects. There is also a need for delta opioid receptor antagonists as immunosuppressants, antiinflammatory agents, agents for the treatment of neurological and psychiatric conditions, agents for the treatment of urological and reproductive conditions, medicaments for drug and alcohol abuse, agents for treating gastritis and diarrhea, cardiovascular agents and agents for the treatment of respiratory diseases, having reduced side effects.

### SUMMARY OF THE INVENTION

The present invention is directed to compounds of Formula (I) and to compositions comprising one or more compounds of Formula (I):

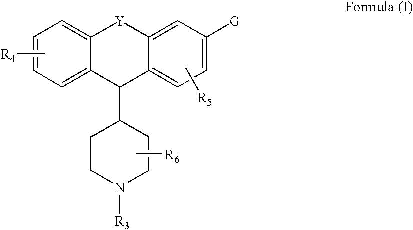

wherein:
G is —C(Z)N(R₁)R₂, C₆₋₁₀aryl, or a heterocycle selected from the group consisting of imidazolyl, triazolyl, tetrazolyl, oxadiazolyl, thiadiazolyl, oxathiadiazolyl, imidazolinyl, tetrahydropyrimidinyl, thienyl, pyrazolyl, pyrimidinyl, triazinyl, furyl, indazolyl, indolyl, indolinyl, isothiazolyl, isoxazolyl, oxazolyl, isoxadiazolyl, benzoxazolyl, quinolinyl, isoquinolinyl, and pyridinyl; wherein aryl and the heterocycles of G are optionally substituted with one to three substituents independently selected from the group consisting of C₁₋₈alkanyl, C₂₋₈alkenyl, C₂₋₈alkynyl, C₁₋₈alkanyloxy, hydroxy(C₁₋₈)alkanyl, carboxy(C₁₋₈)alkanyl, C₁₋₈alkanylcarbonylamino, halogen, hydroxy, cyano, nitro, oxo, thioxo, amino, C₁₋₆alkanylamino, di(C₁₋₆alkanyl)amino, C₁₋₈alkanylthio, C₁₋₈alkanylsulfonyl, C₁₋₈alkanylsulfonylamino, aminocarbonyl, aminothiocarbonyl, aminocarbonylamino, aminothiocarbonylamino, C₁₋₈alkanylaminocarbonyl, di(C₁₋₈alkanyl)aminocarbonyl, and C₁₋₆alkanyloxycarbonylamino;
R₁ is a substituent selected from the group consisting of hydrogen, C₁₋₈alkanyl, C₂₋₈alkenyl, and C₂₋₈alkynyl;
R₂ is a substituent selected from the group consisting of hydrogen; C₁₋₈alkanyl; C₂₋₈alkenyl; C₂₋₈alkynyl; C₆₋₁₀aryl; and C₁₋₈cycloalkanyl; wherein C₁₋₈alkanyl is optionally substituted with one to three substituents independently selected from the group consisting of phenyl, amino, C₁₋₆alkanylamino, di(C₁₋₆alkanyl)amino, C₁₋₆alkanyloxy, thioC₁₋₆alkanyloxy, hydroxy, fluoro, chloro, cyano, aminocarbonyl, C₁₋₈alkanylaminocarbonyl, di(C₁₋₈alkanyl)aminocarbonyl, C₁₋₆alkanyloxycarbonyl, and aryloxy; and wherein any aryl-containing substituents and C₁₋₈cycloalkanyl substituents of R₂ are optionally substituted with one to three substituents independently selected from the group consisting of C₁₋₈alkanyl, C₂₋₈alkenyl, C₂₋₈alkynyl, C₁₋₈alkanyloxy, trifluoromethyl, trifluoromethoxy, phenyl, halogen, cyano, hydroxy, C₁₋₈alkanylthio, C₁₋₈alkanylsulfonyl, and C₁₋₈alkanylsulfonylamino;
or R₁ and R₂ taken together with the nitrogen to which they are attached form a 5-7 membered cycloheteroalkyl optionally substituted with one to three substituents independently selected from the group consisting of C₁₋₈alkanyl, hydroxy(C₁₋₈)alkanyl, hydroxy, amino, C₁₋₆alkanylamino, di(C₁₋₆alkanyl)amino, and halogen;
R₃ is a substituent selected from the group consisting of hydrogen, C₁₋₈alkanyl, halo₁₋₃(C₁₋₈)alkanyl, C₂₋₈alkenyl, C₂₋₈alkynyl, C₃₋₈cycloalkanyl, cycloalkanyl(C₁₋₈)alkanyl, C₁₋₈alkanyloxy(C₁₋₈)alkanyl, C₁₋₈alkanylthio(C₁₋₈)alkanyl, hydroxyC₁₋₈alkanyl, C₁₋₈alkanyloxycarbonyl, halo₁₋₃(C₁₋₈)alkanylcarbonyl, formyl, thioformyl, carbamimidoyl, phenylimino(C₁₋₈)alkanyl, phenyl(C₁₋₈)alkanyl, phenyl(C₁₋₈)alkenyl, phenyl(C₁₋₈)alkynyl, naphthyl(C₁₋₈)alkanyl and heteroaryl(C₁₋₈)alkanyl wherein the heteroaryl is selected from the group consisting of benzo[1,3]dioxolyl, imidazolyl, furanyl, pyridinyl, thienyl, indazolyl, indolyl, indolinyl, isoindolinyl, isoquinolinyl, isothiazolyl, isoxazolyl, oxazolyl, pyrazinyl, pyrazolyl, pyridazinyl, pyrimidinyl, pyrrolyl, quinolinyl, isoquinolinyl, tetrazolyl, thiazolyl; wherein phenyl, naphthyl and heteroaryl are optionally substituted with one to three substituents independently selected from the group consisting of C₁₋₆alkanyl, C₂₋₆alkenyl, C₁₋₆alkanyloxy, amino, C₁₋₆alkanylamino, di(C₁₋₆alkanyl)amino, C₁₋₆alkanylcarbonyl, C₁₋₆alkanylcarbonyloxy, C₁₋₆alkanylcarbonylamino, C₁₋₆alkanylthio, C₁₋₆alkanylsulfonyl, halogen, hydroxy, cyano, fluoro(C₁₋₆)alkanyl, thioureido, and fluoro(C₁₋₆)alkanyloxy; alternatively, when phenyl and heteroaryl are optionally substituted with alkanyl or alkanyloxy substituents attached to adjacent carbon atoms, the two substituents can together form a fused cyclic alkanyl or cycloheteroalkanyl selected from the group consisting of —(CH₂)₃₋₅—, —O(CH₂)₂₋₄—, —(CH₂)₂₋₄O—, and —O(CH₂)₁₋₃O—;
R₄ is one to three substituents independently selected from the group consisting of hydrogen; C₁₋₆alkanyl; C₂₋₆alkenyl; C₂₋₆alkynyl; aryl(C₂₋₆)alkynyl; C₁₋₆alkanyloxy; amino; C₁₋₆alkanylamino; di(C₁₋₆alkanyl)amino; C₆₋₁₀arylamino wherein C₆₋₁₀aryl is optionally substituted with one to three substitutents independently selected from the group consisting of C₁₋₆alkanyl, C₁₋₆alkoxy, halogen, and hydroxyl; formylamino; pyridinylamino; C₁₋₆alkanylcarbonyl; C₁₋₆alkanylcarbonyloxy; C₁₋₆alkanyloxycarbonyl; aminocarbonyl; C₁₋₆alkanylaminocarbonyl; di(C₁₋₆alkanyl)aminocarbonyl; C₁₋₆alkanylcarbonylamino; C₁₋₆alkanylthio; C₁₋₆alkanylsulfonyl; halogen; hydroxy; cyano; hydroxycarbonyl; C₆₋₁₀aryl; chromanyl; chromenyl; furanyl; imidazolyl; indazolyl; indolyl; indolinyl; isoindolinyl; isoquinolinyl; isothiazolyl; isoxazolyl; naphthyridinyl; oxazolyl; pyrazinyl; pyrazolyl; pyridazinyl; pyridinyl; pyrimidinyl; pyrrolyl; quinazolinyl; quinolinyl; quinolizinyl; quinoxalinyl; tetrazolyl; thiazolyl; thienyl; fluoroalkanyl and fluoroalkanyloxy; or optionally; when R₄ is two substituents attached to adjacent carbon atoms, the two substituents together form a single fused moiety, wherein the fused moiety is —(CH₂)₃₋₅—, —O(CH₂)₂₋₄—, —(CH₂)₂₋₄O—, —O(CH₂)₁₋₃O—, or —S—C(NH₂)═N—;
R₅ is one to two substituents independently selected from the group consisting of hydrogen, C₁₋₆alkanyl, C₂₋₆alkenyl, C₁₋₆alkanyloxy, amino, C₁₋₆alkanylamino, di(C₁₋₆alkanyl)amino, C₁₋₆alkanylcarbonyl, C₁₋₆alkanylcarbonyloxy, C₁₋₆alkanyloxycarbonyl, C₁₋₆alkanylaminocarbonyl, C₁₋₆alkanylcarbonylamino, C₁₋₆alkanylthio, C₁₋₆alkanylsulfonyl, halogen, hydroxy, cyano, fluoro(C₁₋₆)alkanyl and fluoro(C₁₋₆)alkanyloxy;
R₆ is one to four substituents independently selected from the group consisting of hydrogen, C₁₋₆alkanyl, C₂₋₆alkenyl, C₁₋₆alkanyloxy, amino, C₁₋₆alkanylamino, di(C₁₋₆alkanyl)amino, C₁₋₆alkanylcarbonyl, C₁₋₆alkanylcarbonyloxy, C₁₋₆alkanyloxycarbonyl, C₁₋₆alkanylaminocarbonyl, C₁₋₆alkanylcarbonylamino, C₁₋₆alkanylthio, C₁₋₆alkanylsulfonyl, halogen, hydroxy, cyano, fluoro(C₁₋₆)alkanyl and fluoro(C₁₋₆)alkanyloxy;
Y is O or S;
Z is O, S, NH, N(C₁₋₆alkanyl), N(OH), N(OC₁₋₆alkanyl), or N(phenyl); and enantiomers, diastereomers, tautomers, solvates, or pharmaceutically acceptable salts thereof.

Finally, the present invention is directed to veterinary and pharmaceutical compositions containing compounds of Formula (I) wherein the compositions are used to treat mild to severe pain in warm-blooded animals.

### DETAILED DESCRIPTION OF THE INVENTION

As used herein, the following underlined terms are intended to have the following meanings:

“C_{a-b}” (where a and b are integers) refers to a radical containing from a to b carbon atoms inclusive. For example, C₁₋₃ denotes a radical containing 1, 2 or 3 carbon atoms

“Alkyl:” refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl”, “alkenyl” and/or “alkynyl” is used, as defined below. In preferred embodiments, the alkyl groups are (C₁-C₆) alkyl, with (C₁-C₃) being particularly preferred.

“Alkanyl:” refers to a saturated branched, straight-chain or cyclic monovalent hydrocarbon radical derived by the removal of one hydrogen atom from a single carbon atom of a parent alkane. Typical alkanyl groups include, but are not limited to, methanyl; ethanyl; propanyls such as propan-1-yl, propan-2-yl, cyclopropan-1-yl, etc.; butyanyls such as butan-1-yl, butan-2-yl, 2-methyl-propan-1-yl, 2-methyl-propan-2-yl, cyclobutan-1-yl, etc.; and the like. In preferred embodiments, the alkanyl groups are (C₁₋₈) alkanyl, with (C₁₋₃) being particularly preferred.

“Alkenyl” refers to an unsaturated branched, straight-chain or cyclic monovalent hydrocarbon radical having at least one carbon-carbon double bond derived by the removal of one hydrogen atom from a single carbon atom of a parent alkene. The radical may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” refers to an unsaturated branched, straight-chain or cyclic monovalent hydrocarbon radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Heteroalkyl” and Heteroalkanyl” refer to alkyl or alkanyl radicals, respectively, in which one or more carbon atoms (and any necessary associated hydrogen atoms) are independently replaced with the same or different heteroatoms (including any necessary hydrogen or other atoms). Typical heteroatoms to replace the carbon atom(s) include, but are not limited to, N, P, O, S, Si, etc. Preferred heteroatoms are O, N and S. Thus, heteroalkanyl radicals can contain one or more of the same or different heteroatomic groups, including, by way of example and not limitation, epoxy (—O—), epidioxy (—O—O—), thioether (—S—), epidithio (—SS—), epoxythio (—O—S—), epoxyimino (—O—NR′—), imino (—NR′—), biimino (—NR′—NR′—), azino (═N—N═), azo (—N═N—), azoxy (—N—O—N—), azimino (—NR′—N═N—), phosphano (—PH—), λ⁴-sulfano (—SH₂—), sulfonyl (—S(O)₂—), and the like, where each R′ is independently hydrogen or (C₁-C₆) alkyl.

“Parent Aromatic Ring System:” refers to an unsaturated cyclic or polycyclic ring system having a conjugated π electron system. Specifically included within the definition of “parent aromatic ring system” are fused ring systems in which one or more rings are aromatic and one or more rings are saturated or unsaturated, such as, for example, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like

“Aryl:” refers to a monovalent aromatic hydrocarbon radical derived by the removal of one hydrogen atom from a single carbon atom of a parent aromatic ring system. Typical aryl groups include, but are not limited to, radicals derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In preferred embodiments, the aryl group is (C₅₋₂₀) aryl, with (C₅₋₁₀) being particularly preferred. Particularly preferred aryl groups are phenyl and naphthyl groups.

“Arylalkyl:” refers to an acyclic alkyl group in which one of the hydrogen atoms bonded to a carbon atom, typically a terminal carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. [In preferred embodiments, the arylalkyl group is (C₆₋₂₆) arylalkyl, e.g., the alkanyl, alkenyl or alkynyl moiety of the arylalkyl group is (C₁₋₆) and the aryl moiety is (C₅₋₂₀). In particularly preferred embodiments the arylalkyl group is (C₆₋₁₃), e.g., the alkanyl, alkenyl or alkynyl moiety of the arylalkyl group is (C₁₋₃) and the aryl moiety is (C₅₋₁₀). Even more preferred arylalkyl groups are phenylalkanyls.

“Alkanyloxy:” refers to a saturated branched, straight-chain or cyclic monovalent hydrocarbon alcohol radical derived by the removal of the hydrogen atom from the hydroxide oxygen of the alcohol. Typical alkanyloxy groups include, but are not limited to, methanyloxy; ethanyloxy; propanyloxy groups such as propan-1-yloxy (CH₃CH₂CH₂O—), propan-2-yloxy ((CH₃)₂CHO—), cyclopropan-1-yloxy, etc.; butanyloxy groups such as butan-1-yloxy, butan-2-yloxy, 2-methyl-propan-1-yloxy, 2-methyl-propan-2-yloxy, cyclobutan-1-yloxy, etc.; and the like. In preferred embodiments, the alkanyloxy groups are (C₁₋₈) alkanyloxy groups, with (C₁₋₃) being particularly preferred.

“Parent Heteroaromatic Ring System:” refers to a parent aromatic ring system in which one carbon atom is replaced with a heteroatom. Heteratoms to replace the carbon atoms include N, O, and S. Specifically included within the definition of “parent heteroaromatic ring systems” are fused ring systems in which one or more rings are aromatic and one or more rings are saturated or unsaturated, such as, for example, arsindole, chromane, chromene, indole, indoline, xanthene, etc. Typical parent heteroaromatic ring systems include, but are not limited to, carbazole, imidazole, indazole, indole, indoline, indolizine, isoindole, isoindoline, isoquinoline, isothiazole, isoxazole, naphthyridine, oxadiazole, oxazole, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl:” refers to a monovalent heteroaromatic radical derived by the removal of one hydrogen atom from a single atom of a parent heteroaromatic ring system. Typical heteroaryl groups include, but are not limited to, radicals derived from carbazole, imidazole, indazole, indole, indoline, indolizine, isoindole, isoindoline, isoquinoline, isothiazole, isoxazole, naphthyridine, oxadiazole, oxazole, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In preferred embodiments, the heteroaryl group is a 5-20 membered heteroaryl, with 5-10 membered heteroaryl being particularly preferred.

“Cycloheteroalkyl:” refers to a saturated or unsaturated monocyclic or bicyclic alkyl radical in which one carbon atom is replaced with N, O or S. In certain specified embodiments the cycloheteroalkyl may contain up to four heteroatoms independently selected from N, O or S. Typical cycloheteroalkyl moieties include, but are not limited to, radicals derived from imidazolidine, morpholine, piperazine, piperidine, pyrazolidine, pyrrolidine, quinuclidine, and the like. In preferred embodiments, the cycloheteroalkyl is a 3-6 membered cycloheteroalkyl.

“Cycloheteroalkanyl:” refers to a saturated monocyclic or bicyclic alkanyl radical in which one carbon atom is replaced with N, O or S. In certain specified embodiments the cycloheteroalkanyl may contain up to four heteroatoms independently selected from N, O or S. Typical cycloheteroalkanyl moieties include, but are not limited to, radicals derived from imidazolidine, morpholine, piperazine, piperidine, pyrazolidine, pyrrolidine, quinuclidine, and the like. In preferred embodiments, the cycloheteroalkanyl is a 3-6 membered cycloheteroalkanyl.

“Cycloheteroalkenyl:” refers to a saturated monocyclic or bicyclic alkenyl radical in which one carbon atom is replaced with N, O or S. In certain specified embodiments the cycloheteroalkenyl may contain up to four heteroatoms independently selected from N, O or S. Typical cycloheteroalkenyl moieties include, but are not limited to, radicals derived from imidazoline, pyrazoline, pyrroline, indoline, pyran, and the like. In preferred embodiments, the cycloheteroalkanyl is a 3-6 membered cycloheteroalkanyl.

“Substituted:” refers to a radical in which one or more hydrogen atoms are each independently replaced with the same or different substituent(s). Typical substituents include, but are not limited to, —X, —R, —O—, ═O, —OR, —O—OR, —SR, —S—, ═S, —NRR, ═NR, —CX₃, —CN, —OCN, —SCN, —NCO, —NCS, —NO, —NO₂, ═N₂, —N₃, —NHOH, —S(O)₂₋₀—, —S(O)₂OH, —S(O)₂R, —P(O)(O—)₂, —P(O)(OH)₂, —C(O)R, —C(O)X, —C(S)R, —C(S)X, —C(O)OR, —C(O)O⁻, —C(S)OR, —C(O)SR, —C(S)SR, —C(O)NRR, —C(S)NRR and —C(NR)NRR, where each X is independently a halogen (preferably —F, —Cl or —Br) and each R is independently —H, alkyl, alkanyl, alkenyl, alkynyl, alkylidene, alkylidyne, aryl, arylalkyl, arylheteroalkyl, heteroaryl, heteroarylalkyl or heteroaryl-heteroalkyl, as defined herein. Preferred substituents include hydroxy, halogen, C₁₋₈alkyl, C₁₋₈alkanyloxy, fluorinated alkanyloxy, fluorinated alkyl, C₁₋₈alkylthio, C₃₋₈cycloalkyl, C₃₋₈cycloalkanyloxy, nitro, amino, C₁₋₈alkylamino, C₁₋₈dialkylamino, C₃₋₈cycloalkylamino, cyano, carboxy, C₁₋₇alkanyloxycarbonyl, C₁₋₇alkylcarbonyloxy, formyl, carbamoyl, phenyl, aroyl, carbamoyl, amidino, (C₁₋₈alkylamino)carbonyl, (arylamino)carbonyl and aryl(C₁₋₈alkyl)carbonyl.

With reference to substituents, the term “independently” means that when more than one of such substituent is possible, such substituents may be the same or different from each other.

Throughout this disclosure, the terminal portion of the designated side chain is described first, followed by the adjacent functionality toward the point of attachment. Thus, for example, a “phenylC₁₋₆alkanylaminocarbonylC₁₋₆alkyl” substituent refers to a group of the formula

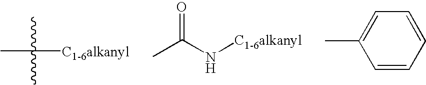

An embodiment of the present invention is directed to compounds of Formula (I) wherein the structure of Formula (I) is as defined below.

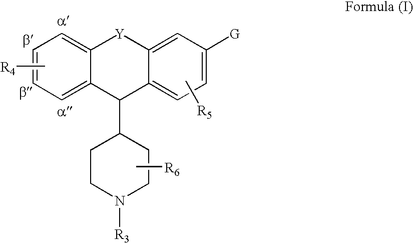

The present invention is directed to analgesic and anti-pyretic uses of compositions comprising a compound of Formula (I):

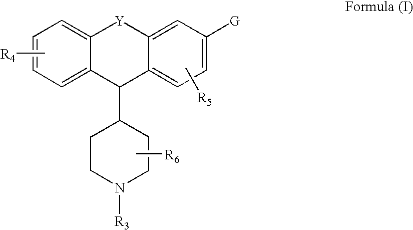

wherein:
G is —C(Z)N(R₁)R₂, C₆₋₁₀aryl, or a heterocycle selected from the group consisting of: imidazolyl, triazolyl, tetrazolyl, oxadiazolyl, thiadiazolyl, oxathiadiazolyl, imidazolinyl, tetrahydropyrimidinyl, thienyl, pyrazolyl, pyrimidinyl, triazinyl, furyl, indazolyl, indolyl, indolinyl, isothiazolyl, isoxazolyl, oxazolyl, isoxadiazolyl, benzoxazolyl, quinolinyl, isoquinolinyl, and pyridinyl; wherein aryl and the heterocycles of G are optionally substituted with one to three substituents independently selected from the group consisting of C₁₋₈alkanyl, C₂₋₈alkenyl, C₂₋₈alkynyl, C₁₋₈alkanyloxy, hydroxy(C₁₋₈)alkanyl, carboxy(C₁₋₈)alkanyl, C₁₋₈alkanylcarbonylamino, halogen, hydroxy, cyano, nitro, oxo, thioxo, amino, C₁₋₆alkanylamino, di(C₁₋₆alkanyl)amino, C₁₋₈alkanylthio, C₁₋₈alkanylsulfonyl, C₁₋₈alkanylsulfonylamino, aminocarbonyl, aminothiocarbonyl, aminocarbonylamino, aminothiocarbonylamino, C₁₋₈alkanylaminocarbonyl, di(C₁₋₈alkanyl)aminocarbonyl, and C₁₋₆alkanyloxycarbonylamino;
R₁ is a substituent selected from the group consisting of hydrogen, C₁₋₈alkanyl, C₂₋₈alkenyl, and C₂₋₈alkynyl;
R₂ is a substituent selected from the group consisting of hydrogen; C₁₋₈alkanyl; C₂₋₈alkenyl; C₂₋₈alkynyl; C₆₋₁₀aryl; and C₁₋₈cycloalkanyl; wherein C₁₋₈alkanyl is optionally substituted with one to three substituents independently selected from the group consisting of phenyl, amino, C₁₋₆alkanylamino, di(C₁₋₆alkanyl)amino, C₁₋₆alkanyloxy, thioC₁₋₆alkanyloxy, hydroxy, fluoro, chloro, cyano, aminocarbonyl, C₁₋₈alkanylaminocarbonyl, di(C₁₋₈alkanyl)aminocarbonyl, C₁₋₆alkanyloxycarbonyl, and aryloxy; and wherein any aryl-containing substituents and C₁₋₈cycloalkanyl substituents of R₂ are optionally substituted with one to three substituents independently selected from the group consisting of C₁₋₈alkanyl, C₂₋₈alkenyl, C₂₋₈alkynyl, C₁₋₈alkanyloxy, trifluoromethyl, trifluoromethoxy, phenyl, halogen, cyano, hydroxy, C₁₋₈alkanylthio, C₁₋₈alkanylsulfonyl, and C₁₋₈alkanylsulfonylamino;
or R₁ and R₂ taken together with the nitrogen to which they are attached form a 5-7 membered cycloheteroalkyl optionally substituted with one to three substituents independently selected from the group consisting of C₁₋₈alkanyl, hydroxy(C₁₋₈)alkanyl, hydroxy, amino, C₁₋₆alkanylamino, di(C₁₋₆alkanyl)amino, and halogen;
R₃ is a substituent selected from the group consisting of hydrogen, C₁₋₈alkanyl, halo₁₋₃(C₁₋₈)alkanyl, C₂₋₈alkenyl, C₂₋₈alkynyl, C₃₋₈cycloalkanyl, cycloalkanyl(C₁₋₈)alkanyl, C₁₋₈alkanyloxy(C₁₋₈)alkanyl, C₁₋₈alkanylthio(C₁₋₈)alkanyl, hydroxyC₁₋₈alkanyl, C₁₋₈alkanyloxycarbonyl, halo₁₋₃(C₁₋₈)alkanylcarbonyl, formyl, thioformyl, carbamimidoyl, phenylimino(C₁₋₈)alkanyl, phenyl(C₁₋₈)alkanyl, phenyl(C₁₋₈)alkenyl, phenyl(C₁₋₈)alkynyl, naphthyl(C₁₋₈)alkanyl and heteroaryl(C₁₋₈)alkanyl wherein the heteroaryl is selected from the group consisting of benzo[1,3]dioxolyl, imidazolyl, furanyl, pyridinyl, thienyl, indazolyl, indolyl, indolinyl, isoindolinyl, isoquinolinyl, isothiazolyl, isoxazolyl, oxazolyl, pyrazinyl, pyrazolyl, pyridazinyl, pyrimidinyl, pyrrolyl, quinolinyl, isoquinolinyl, tetrazolyl, thiazolyl; wherein phenyl, naphthyl and heteroaryl are optionally substituted with one to three substituents independently selected from the group consisting of C₁₋₆alkanyl, C₂₋₆alkenyl, C₁₋₆alkanyloxy, amino, C₁₋₆alkanylamino, di(C₁₋₆alkanyl)amino, C₁₋₆alkanylcarbonyl, C₁₋₆alkanylcarbonyloxy, C₁₋₆alkanylcarbonylamino, C₁₋₆alkanylthio, C₁₋₆alkanylsulfonyl, halogen, hydroxy, cyano, fluoro(C₁₋₆)alkanyl, thioureido, and fluoro(C₁₋₆)alkanyloxy; alternatively, when phenyl and heteroaryl are optionally substituted with alkanyl or alkanloxy substituents attached to adjacent carbon atoms, the two substituents can together form a fused cyclic alkanyl or cycloheteroalkanyl selected from the group consisting of —(CH₂)₃₋₅—, —O(CH₂)₂₋₄—, —(CH₂)₂₋₄O—, and —O(CH₂)₁₋₃O—;
R₄ is one to three substituents independently selected from the group consisting of hydrogen; C₁₋₆alkanyl; C₂₋₆alkenyl; C₂₋₆alkynyl; aryl(C₂₋₆)alkynyl; C₁₋₆alkanyloxy; amino; C₁₋₆alkanylamino; di(C₁₋₆alkanyl)amino; C₆₋₁₀arylamino wherein C₆₋₁₀aryl is optionally substituted with one to three substitutents independently selected from the group consisting of C₁₋₆alkanyl, C₁₋₆alkoxy, halogen, and hydroxy; formylamino; pyridinylamino; C₁₋₆alkanylcarbonyl; C₁₋₆alkanylcarbonyloxy; C₁₋₆alkanyloxycarbonyl; aminocarbonyl; C₁₋₆alkanylaminocarbonyl; di(C₁₋₆alkanyl)aminocarbonyl; C₁₋₆alkanylcarbonylamino; C₁₋₆alkanylthio; C₁₋₆alkanylsulfonyl; halogen; hydroxy; cyano; hydroxycarbonyl; C₆₋₁₀aryl; chromanyl; chromenyl; furanyl; imidazolyl; indazolyl; indolyl; indolinyl; isoindolinyl; isoquinolinyl; isothiazolyl; isoxazolyl; naphthyridinyl; oxazolyl; pyrazinyl; pyrazolyl; pyridazinyl; pyridinyl; pyrimidinyl; pyrrolyl; quinazolinyl; quinolinyl; quinolizinyl; quinoxalinyl; tetrazolyl; thiazolyl; thienyl; fluoroalkanyl and fluoroalkanyloxy; or optionally, when R₄ is two substituents attached to adjacent carbon atoms, the two substituents together form a single fused moiety, wherein the fused moiety is —(CH₂)₃₋₅—, —O(CH₂)₂₋₄—(CH₂)₂₋₄O—, —O(CH₂)₁₋₃O—, or —S—C(NH₂)═N—;
R₅ is one to two substituents independently selected from the group consisting of hydrogen, C₁₋₆alkanyl, C₂₋₆alkenyl, C₁₋₆alkanyloxy, amino, C₁₋₆alkanylamino, di(C₁₋₆alkanyl)amino, C₁₋₆alkanylcarbonyl, C₁₋₆alkanylcarbonyloxy, C₁₋₆alkanyloxycarbonyl, C₁₋₆alkanylaminocarbonyl, C₁₋₆alkanylcarbonylamino, C₁₋₆alkanylthio, C₁₋₆alkanylsulfonyl, halogen, hydroxy, cyano, fluoro(C₁₋₆)alkanyl and fluoro(C₁₋₆)alkanyloxy;
R₆ is one to four substituents independently selected from the group consisting of hydrogen, C₁₋₆alkanyl, C₂₋₆alkenyl, C₁₋₆alkanyloxy, amino, C₁₋₆alkanylamino, di(C₁₋₆alkanyl)amino, C₁₋₆alkanylcarbonyl, C₁₋₆alkanylcarbonyloxy, C₁₋₆alkanyloxycarbonyl, C₁₋₆alkanylaminocarbonyl, C₁₋₆alkanylcarbonylamino, C₁₋₆alkanylthio, C₁₋₆alkanylsulfonyl, halogen, hydroxy, cyano, fluoro(C₁₋₆)alkanyl and fluoro(C₁₋₆)alkanyloxy;
Y is O or S;
Z is O, S, NH, N(C₁₋₆alkanyl), N(OH), N(OC₁₋₆alkanyl), or N(phenyl); and enantiomers, diastereomers, tautomers, solvates, or pharmaceutically acceptable salts thereof.

Embodiments of the present invention include compounds of Formula (J) wherein, preferably:
a) G is —C(Z)N(R₁)R₂, phenyl, or a heterocycle selected from the group consisting of imidazolyl, triazolyl, tetrazolyl, oxadiazolyl, thiadiazolyl, oxathiadiazolyl, imidazolinyl, tetrahydropyrimidinyl, thienyl, pyrazolyl, pyrimidinyl, triazinyl, isothiazolyl, isoxazolyl, oxazolyl, isoxadiazolyl, and pyridinyl; wherein phenyl and the heterocycles of G are optionally substituted with one to three substituents independently selected from the group consisting of C₁₋₈alkanyl, C₁₋₈alkanyloxy, hydroxy(C₁₋₈)alkanyl, carboxy(C₁₋₈)alkanyl, C₁₋₈alkanylcarbonylamino, halogen, hydroxy, cyano, oxo, thioxo, amino, C₁₋₆alkanylamino, di(C₁₋₆alkanyl)amino, C₁₋₈alkanylthio, aminocarbonyl, aminothiocarbonyl, C₁₋₈alkanylaminocarbonyl, di(C₁₋₈alkanyl)aminocarbonyl, and C₁₋₆alkanyloxycarbonylamino;
b) G is —C(Z)N(R₁)R₂, phenyl, or a heterocycle selected from the group consisting of imidazolyl, tetrazolyl, oxadiazolyl, thiadiazolyl, oxathiadiazolyl, imidazolinyl, thienyl, pyrazolyl, pyrimidinyl, triazinyl, isothiazolyl, isoxazolyl, oxazolyl, isoxadiazolyl, and pyridinyl; wherein phenyl and the heterocycles of G (described herein) are optionally substituted with one to three substituents independently selected from the group consisting of C₁₋₄alkanyl, C₁₋₄alkanyloxy, hydroxy(C₁₋₄)alkanyl, carboxy(C₁₋₄)alkanyl, C₁₋₄alkanylcarbonylamino, hydroxy, cyano, oxo, thioxo, amino, C₁₋₆alkanylamino, di(C₁₋₆alkanyl)amino, C₁₋₈alkanylthio, aminocarbonyl, aminothiocarbonyl, C₁₋₈alkanylaminocarbonyl, and di(C₁₋₈alkanyl)aminocarbonyl;
c) G is —C(Z)N(R₁)R₂, phenyl, or a heterocycle selected from the group consisting of imidazolyl, tetrazolyl, oxadiazolyl, thiadiazolyl, oxathiadiazolyl, thienyl, isothiazolyl, isoxazolyl, isoxadiazolyl, and pyridinyl; wherein phenyl and the heterocycles of G (described herein) are optionally substituted with one to three substituents independently selected from the group consisting of C₁₋₄alkanyl, C₁₋₄alkanyloxy, hydroxy(C₁₋₄)alkanyl, C₁₋₄alkanylcarbonylamino, hydroxy, cyano, oxo, thioxo, and aminocarbonyl;
d) G is —C(Z)N(R₁)R₂, phenyl, or a heterocycle selected from the group consisting of tetrazolyl, oxadiazolyl, and pyridinyl; wherein phenyl and the heterocycles of G are optionally substituted with one to three substituents independently selected from the group consisting of C₁₋₄alkanylcarbonylamino and oxo;
e) G is N,N-diethylaminocarbonyl, 3-(N,N-diethylaminocarbonyl)-phenyl, 2-methylcarbonylaminophenyl, N—N-diethylamidino, pyridin-3-yl, 3-(S)hydroxypyrrolidin-1-ylcarbonyl, N-ethylaminocarbonyl, 1H-tetrazol-4-yl, pyridine-4-yl, or 4H-[1,2,4]-oxadiazol-5-oxo-3-yl;
f) R₁ is a substituent selected from the group consisting of hydrogen and C₁₋₄alkanyl;
g) R₁ is selected from the group consisting of hydrogen, methyl, ethyl, and propyl;
h) R₁ is selected from the group consisting of hydrogen, methyl, or ethyl;
i) R₁ is selected from the group consisting of hydrogen and ethyl;
j) R₂ is selected from the group consisting of hydrogen; C₁₋₄alkanyl; phenyl; and C₁₋₆cycloalkanyl; wherein C₁₋₄alkanyl is optionally substituted with one to three substituents independently selected from the group consisting of phenyl, amino, C₁₋₆alkanylamino, di(C₁₋₆alkanyl)amino, C₁₋₄alkanyloxy, hydroxy, fluoro, chloro, cyano, aminocarbonyl, C₁₋₈alkanylaminocarbonyl, di(C₁₋₈alkanyl)aminocarbonyl, and phenoxy; and wherein any phenyl-containing substituents and C₁₋₆cycloalkanyl substituents of R₂ are optionally substituted with one to three substituents independently selected from the group consisting of C₁₋₈alkanyl, C₁₋₈alkanyloxy, trifluoromethyl, phenyl, fluoro, hydroxy, C₁₋₈alkanylthio, C₁₋₈alkanylsulfonyl, and C₁₋₈alkanylsulfonylamino; or R₁ and R₂ taken together with the nitrogen to which they are attached form a 5-7 membered cycloheteroalkyl optionally substituted with one to three substituents independently selected from the group consisting of C₁₋₄alkanyl, hydroxy(C₁₋₄)alkanyl, hydroxy, amino, C₁₋₆alkanylamino, di(C₁₋₆alkanyl)amino, and fluoro;
k) R₂ is selected from the group consisting of hydrogen, C₁₋₄alkanyl, phenyl, and C₁₋₆cycloalkanyl, wherein C₁₋₄alkanyl is optionally substituted with one to three substituents independently selected from the group consisting of phenyl, C₁₋₄alkanyloxy, hydroxy, fluoro, aminocarbonyl, C₁₋₈alkanylaminocarbonyl, di(C₁₋₈alkanyl)aminocarbonyl, and phenoxy; and wherein any phenyl-containing substituent of R₂ is optionally substituted with one to three substituents independently selected from the group consisting of C₁₋₆alkanyl, C₁₋₆alkanyloxy, fluoro, hydroxy, and C₁₋₆alkanylthio; or R₁ and R₂ taken together with the nitrogen to which they are attached form a 5-7 membered cycloheteroalkyl optionally substituted with one to three substituents independently selected from the group consisting of C₁₋₄alkanyl and hydroxy;
l) R₂ is selected from the group consisting of hydrogen, C₁₋₄alkanyl and phenyl, wherein C₁₋₄alkanyl is optionally substituted with one to three substituents independently selected from the group consisting of phenyl, C₁₋₄alkanyloxy, hydroxy, fluoro, and phenoxy; and wherein any phenyl-containing substituent of R₂ is optionally substituted with one to three substituents independently selected from the group consisting of C₁₋₆alkanyl, C₁₋₆alkanyloxy, fluoro, and hydroxy; or R₁ and R₂ taken together with the nitrogen to which they are attached form a pyrrolidinyl or piperidinyl ring wherein said pyrrolidinyl or piperidinyl is optionally substituted with a substituent selected from the group consisting of C₁₋₃alkanyl and hydroxy;
m) R₂ is selected from the group consisting of hydrogen and C₁₋₄alkanyl, or R₁ and R₂ taken together with the nitrogen to which they are attached form a pyrrolidinyl ring optionally substituted with hydroxy;
n) R₂ is hydrogen or ethyl, or R₁ and R₂ taken together with the nitrogen to which they are attached form a pyrrolidinyl ring optionally substituted with hydroxy;
o) R₃ is selected from the group consisting of hydrogen, C₁₋₈alkanyl, C₂₋₈alkenyl, C₂₋₈alkynyl, C₁₋₈alkanyloxy(C₁₋₈)alkanyl, C₁₋₈alkanylthio(C₁₋₈)alkanyl, hydroxyC₁₋₈alkanyl, thioformyl, phenylimino(C₁₋₈)alkanyl, phenyl(C₁₋₈)alkanyl, and heteroaryl(C₁₋₈)alkanyl wherein heteroaryl is selected from the group consisting of benzo[1,3]dioxolyl, imidazolyl, furanyl, pyridinyl, thienyl, indolyl, indolinyl, isoquinolinyl, pyrazinyl, pyrazolyl, pyridazinyl, pyrimidinyl, pyrrolyl, quinolinyl, isoquinolinyl, tetrazolyl; wherein phenyl and heteroaryl are optionally substituted with one to three substituents independently selected from the group consisting of C₁₋₆alkanyloxy and hydroxy; or optionally, when phenyl and heteroaryl are optionally substituted with two substituents attached to adjacent carbon atoms, the two substituents together form a single fused moiety; wherein the moiety is selected from —O(CH₂)₁₋₃O—;
p) R₃ is selected from the group consisting of hydrogen, methyl, allyl, 2-methyl-allyl, propynyl, hydroxyethyl, methylthioethyl, methoxyethyl, thioformyl, phenyliminomethyl, phenethyl, and heteroaryl(C₁₋₈)alkanyl wherein the heteroaryl is selected from the group consisting of benzo[1,3]dioxolyl, imidazolyl, furanyl, pyridinyl, thienyl, pyrimidinyl, pyrrolyl, quinolinyl, isoquinolinyl, tetrazolyl; wherein the phenyl in any phenyl-containing substituent is optionally substituted with one hydroxyl group;
q) R₃ is hydrogen, C₁₋₈alkanyl, C₂₋₈alkenyl, C₃₋₈cycloalkanyl, phenyl(C₁₋₈)alkanyl, or heteroaryl(C₁₋₈)alkanyl wherein the heteroaryl is imidazolyl, furanyl, pyridinyl, or thienyl;
r) R₃ is hydrogen, methyl, allyl, or heteroarylmethyl wherein heteroaryl is selected from the group consisting of benzo[1,3]dioxolyl, imidazolyl, furanyl, pyridinyl, and thienyl;
s) R₃ is hydrogen, methyl, ethyl, propenyl, cyclopropylmethyl, benzyl, phenethyl, or heteroarylmethyl wherein the heteroaryl is furanyl, imidazolyl, pyridinyl, or thienyl;
t) R₄ is one to three substituents independently selected from the group consisting of hydrogen; C₁₋₆alkanyl; C₁₋₆alkanyloxy; C₆₋₁₀arylamino wherein C₆₋₁₀aryl is optionally substituted with one to three substitutents independently selected from the group consisting of C₁₋₆alkanyl, C₁₋₆alkoxy, halogen, and hydroxy; formylamino; pyridinylamino; aminocarbonyl; C₁₋₆alkanylaminocarbonyl; C₁₋₆alkanylcarbonylamino; halogen; hydroxy; C₆₋₁₀aryl; chromanyl; chromenyl; furanyl; imidazolyl; indazolyl; indolyl; indolinyl; isoindolinyl; isoquinolinyl; isothiazolyl; isoxazolyl; naphthyridinyl; oxazolyl; pyrazinyl; pyrazolyl; pyridazinyl; pyridinyl; pyrimidinyl; pyrrolyl; quinazolinyl; quinolinyl; quinolizinyl; quinoxalinyl; tetrazolyl; thiazolyl; and thienyl;
u) R₄ is one to two substituents independently selected from the group consisting of hydrogen, C₁₋₄alkanyl, C₁₋₄alkanyloxy, halogen, phenyl, furanyl, imidazolyl, indazolyl, indolyl, indolinyl, isoindolinyl, isoquinolinyl, isothiazolyl, isoxazolyl, oxazolyl, pyrazinyl, pyrazolyl, pyridazinyl, pyridinyl, pyrimidinyl, pyrrolyl, quinolinyl, tetrazolyl, thiazolyl, thienyl, and hydroxy;
v) R₄ is one to two substituents independently selected from the group consisting of hydrogen, methyl, methoxy, bromo, fluoro, α′- or β′-phenyl, α′- or β′-pyridinyl, α′- or β′-furanyl, and hydroxy;
w) R₄ is one substituent selected from the group consisting of hydrogen, methoxy, chloro, and hydroxy;
x) R₄ is one substituent and is hydrogen or hydroxy;
y) R₅ is one to two substituents independently selected from the group consisting of hydrogen and halogen;
z) R₅ is hydrogen;
aa) R₆ is one to four substituents independently selected from the group consisting of hydrogen, C₁₋₆alkanyl, C₂₋₆alkenyl, C₁₋₆alkanyloxy, halogen, hydroxy, fluoro(C₁₋₆)alkanyl and fluoro(C₁₋₆)alkanyloxy;
bb) R₆ is one to two substituents independently selected from the group consisting of hydrogen and C₁₋₄alkanyl;
cc) R₆ is one to two substituents independently selected from the group consisting of hydrogen and methyl;
dd) R₆ is hydrogen;
ee) Y is O or S;
ff) Y is O;
gg) Z is O, NH, N(C₁₋₆alkanyl), N(OH), N(OC₁₋₆alkanyl), or N(phenyl);
hh) Z is O, NH, or N(OH);
ii) Z is O or NH;
jj) Z is O;
kk) R₄ is hydrogen and Y is O;
ll) R₄ is α′-hydroxy and Y is O;
mm) R₄ is hydrogen and Y is S;
nn) R₄ is α′-hydroxyl and Y is S;
   and combinations of a) through x) above.

One embodiment of the present invention is a compound of Formula (J) wherein:
G is —C(Z)N(R₁)R₂, phenyl, or a heterocycle selected from the group consisting of imidazolyl, triazolyl, tetrazolyl, oxadiazolyl, thiadiazolyl, oxathiadiazolyl, imidazolinyl, tetrahydropyrimidinyl, thienyl, pyrazolyl, pyrimidinyl, triazinyl, isothiazolyl, isoxazolyl, oxazolyl, isoxadiazolyl, and pyridinyl; wherein phenyl and the heterocycles of G are optionally substituted with one to three substituents independently selected from the group consisting of C₁₋₈alkanyl, C₁₋₈alkanyloxy, hydroxy(C₁₋₈)alkanyl, carboxy(C₁₋₈)alkanyl, C₁₋₈alkanylcarbonylamino, halogen, hydroxy, cyano, oxo, thioxo, amino, C₁₋₆alkanylamino, di(C₁₋₆alkanyl)amino, C₁₋₈alkanylthio, aminocarbonyl, aminothiocarbonyl, C₁₋₈alkanylaminocarbonyl, di(C₁₋₈alkanyl)aminocarbonyl, and C₁₋₆alkanyloxycarbonylamino;
R₁ is hydrogen or C₁₋₄alkanyl;
R₂ is selected from the group consisting of hydrogen; C₁₋₄alkanyl; phenyl; and C₁₋₆cycloalkanyl; wherein C₁₋₄alkanyl is optionally substituted with one to three substituents independently selected from the group consisting of phenyl, amino, C₁₋₆alkanylamino, di(C₁₋₆alkanyl)amino, C₁₋₄alkanyloxy, hydroxy, fluoro, chloro, cyano, aminocarbonyl, C₁₋₈alkanylaminocarbonyl, di(C₁₋₈alkanyl)aminocarbonyl, and phenoxy; and wherein the phenyl and C₁₋₆cycloalkanyl substituents of R₂ are optionally substituted with one to three substituents independently selected from the group consisting of C₁₋₈alkanyl, C₁₋₈alkanyloxy, trifluoromethyl, phenyl, fluoro, hydroxy, C₁₋₈alkanylthio, C₁₋₈alkanylsulfonyl, and C₁₋₈alkanylsulfonylamino; or R₁ and R₂ taken together with the nitrogen to which they are attached form a 5-7 membered cycloheteroalkyl optionally substituted with one to three substituents independently selected from the group consisting of C₁₋₄alkanyl, hydroxy(C₁₋₄)alkanyl, hydroxy, amino, C₁₋₆alkanylamino, di(C₁₋₆alkanyl)amino, and fluoro;
R₃ is selected from the group consisting of hydrogen, C₁₋₈alkanyl, C₂₋₈alkenyl, C₂₋₈alkynyl, C₁₋₈alkanyloxy(C₁₋₈)alkanyl, C₁₋₈alkanylthio(C₁₋₈)alkanyl, hydroxyC₁₋₈alkanyl, thioformyl, phenylimino(C₁₋₈)alkanyl, phenyl(C₁₋₈)alkanyl, and heteroaryl(C₁₋₈)alkanyl wherein heteroaryl is selected from the group consisting of benzo[1,3]dioxolyl, imidazolyl, furanyl, pyridinyl, thienyl, indolyl, indolinyl, isoquinolinyl, pyrazinyl, pyrazolyl, pyridazinyl, pyrimidinyl, pyrrolyl, quinolinyl, isoquinolinyl, tetrazolyl; wherein phenyl and heteroaryl are optionally substituted with one to three substituents independently selected from the group consisting of C₁₋₆alkanyloxy and hydroxy; or optionally, when phenyl and heteroaryl are optionally substituted with two substituents attached to adjacent carbon atoms, the two substituents together form a single fused moiety; wherein the moiety is selected from —O(CH₂)₁₋₃O—;
R₄ is one to three substituents independently selected from the group consisting of hydrogen; C₁₋₆alkanyl; C₁₋₆alkanyloxy; C₆₋₁₀arylamino wherein C₆₋₁₀aryl is optionally substituted with one to three substitutents independently selected from the group consisting of C₁₋₆alkanyl, C₁₋₆alkoxy, halogen, and hydroxy; formylamino; pyridinylamino; aminocarbonyl; C₁₋₆alkanylaminocarbonyl; C₁₋₆alkanylcarbonylamino; halogen; hydroxy; C₆₋₁₀aryl; chromanyl; chromenyl; furanyl; imidazolyl; indazolyl; indolyl; indolinyl; isoindolinyl; isoquinolinyl; isothiazolyl; isoxazolyl; naphthyridinyl; oxazolyl; pyrazinyl; pyrazolyl; pyridazinyl; pyridinyl; pyrimidinyl; pyrrolyl; quinazolinyl; quinolinyl; quinolizinyl; quinoxalinyl; tetrazolyl; thiazolyl; and thienyl;
R₅ is one to two substituents independently selected from the group consisting of hydrogen and halogen;
R₆ is one to four substituents independently selected from the group consisting of hydrogen, C₁₋₆alkanyl, C₂₋₆alkenyl, C₁₋₆alkanyloxy, halogen, hydroxy, fluoro(C₁₋₆)alkanyl and fluoro(C₁₋₆)alkanyloxy;
Y is O or S;
Z is O, NH, N(C₁₋₆alkanyl), N(OH), N(OC₁₋₆alkanyl), or N(phenyl); and enantiomers, diastereomers, tautomers, solvates, and pharmaceutically acceptable salts thereof.

Another embodiment of the present invention is a compound of Formula (I) wherein:
G is —C(Z)N(R₁)R₂, phenyl, or a heterocycle selected from the group consisting of imidazolyl, tetrazolyl, oxadiazolyl, thiadiazolyl, oxathiadiazolyl, imidazolinyl, thienyl, pyrazolyl, pyrimidinyl, triazinyl, isothiazolyl, isoxazolyl, oxazolyl, isoxadiazolyl, and pyridinyl; wherein phenyl and the heterocycles of G (described herein) are optionally substituted with one to three substituents independently selected from the group consisting of C₁₋₄alkanyl, C₁₋₄alkanyloxy, hydroxy(C₁₋₄)alkanyl, carboxy(C₁₋₄)alkanyl, C₁₋₄alkanylcarbonylamino, hydroxy, cyano, oxo, thioxo, amino, C₁₋₆alkanylamino, di(C₁₋₆alkanyl)amino, C₁₋₈alkanylthio, aminocarbonyl, aminothiocarbonyl, C₁₋₈alkanylaminocarbonyl, and di(C₁₋₈alkanyl)aminocarbonyl;
R₁ is selected from the group consisting of hydrogen, methyl, ethyl, and propyl;
R₂ is selected from the group consisting of hydrogen, C₁₋₄alkanyl, phenyl, and C₁₋₆cycloalkanyl; wherein C₁₋₄alkanyl is optionally substituted with one to three substituents independently selected from the group consisting of phenyl, C₁₋₄alkanyloxy, hydroxy, fluoro, aminocarbonyl, C₁₋₈alkanylaminocarbonyl, di(C₁₋₈alkanyl)aminocarbonyl, and phenoxy; and wherein any phenyl-containing substituent of R₂ is optionally substituted with one to three substituents independently selected from the group consisting of C₁₋₆alkanyl, C₁₋₆alkanyloxy, fluoro, hydroxy, and C₁₋₆alkanylthio; or R₁ and R₂ taken together with the nitrogen to which they are attached form a pyrrolidinyl or piperidinyl ring wherein said pyrrolidinyl or piperidinyl is optionally substituted with a substituent selected from the group consisting of C₁₋₃alkanyl and hydroxy;
R₃ is selected from the group consisting of hydrogen, methyl, allyl, 2-methyl-allyl, propynyl, hydroxyethyl, methylthioethyl, methoxyethyl, thioformyl, phenyliminomethyl, phenethyl, and heteroaryl(C₁₋₈)alkanyl wherein the heteroaryl is selected from the group consisting of benzo[1,3]dioxolyl, imidazolyl, furanyl, pyridinyl, thienyl, pyrimidinyl, pyrrolyl, quinolinyl, isoquinolinyl, tetrazolyl wherein the phenyl in any phenyl-containing substituent is optionally substituted with one hydroxyl group;
R₄ is one to two substituents independently selected from the group consisting of hydrogen, C₁₋₄alkanyl, C₁₋₄alkanyloxy, halogen, phenyl, furanyl, imidazolyl, indazolyl, indolyl, indolinyl, isoindolinyl, isoquinolinyl, isothiazolyl, isoxazolyl, oxazolyl, pyrazinyl, pyrazolyl, pyridazinyl, pyridinyl, pyrimidinyl, pyrrolyl, quinolinyl, tetrazolyl, thiazolyl, thienyl, and hydroxy;
R₅ is hydrogen;
R₆ is one to two substituents independently selected from the group consisting of hydrogen and C₁₋₄alkanyl;
Y is O or S;
Z is O, NH, or N(OH); and
enantiomers, diastereomers, tautomers, solvates, and pharmaceutically acceptable salts thereof.

Another embodiment of the present invention is directed to compositions comprising a compound of Formula (J) wherein:
G is selected from —C(Z)N(R₁)R₂, phenyl, or a heterocycle selected from the group consisting of imidazolyl, tetrazolyl, oxadiazolyl, thiadiazolyl, oxathiadiazolyl, thienyl, isothiazolyl, isoxazolyl, isoxadiazolyl, and pyridinyl; wherein phenyl and the heterocycles of G are optionally substituted with one to three substituents independently selected from the group consisting of C₁₋₄alkanyl, C₁₋₄alkanyloxy, hydroxy(C₁₋₄)alkanyl, C₁₋₄alkanylcarbonylamino, hydroxy, cyano, oxo, thioxo, and aminocarbonyl; R₁ is hydrogen, methyl, or ethyl;
R₂ is independently selected from the group consisting of hydrogen, C₁₋₄alkanyl and phenyl; wherein C₁₋₄alkanyl is optionally substituted with one to three substituents independently selected from the group consisting of phenyl, C₁₋₄alkanyloxy, hydroxy, fluoro, and phenoxy; and wherein any phenyl-containing substituent of R₂ is optionally substituted with one to three substituents independently selected from the group consisting of C₁₋₆alkanyl, C₁₋₆alkanyloxy, fluoro, and hydroxy; or R₁ and
R₂ taken together with the nitrogen to which they are attached form a pyrrolidinyl or piperidinyl ring wherein said pyrrolidinyl or piperidinyl are optionally substituted with a substituent selected from the group consisting of C₁₋₃alkanyl and hydroxy;
R₃ is hydrogen, methyl, allyl, or heteroarylmethyl wherein heteroaryl is selected from the group consisting of benzo[1,3]dioxolyl, imidazolyl, furanyl, pyridinyl, and thienyl;
R₄ is one to two substituents independently selected from the group consisting of hydrogen, C₁₋₄alkanyl, C₁₋₄alkanyloxy, halogen, phenyl, furanyl, imidazolyl, indazolyl, indolyl, indolinyl, isoindolinyl, isoquinolinyl, isothiazolyl, isoxazolyl, oxazolyl, pyrazinyl, pyrazolyl, pyridazinyl, pyridinyl, pyrimidinyl, pyrrolyl, quinolinyl, tetrazolyl, thiazolyl, thienyl, and hydroxy;
R₆ is one to two substituents independently selected from the group consisting of hydrogen and methyl;
Y is O or S;
Z is O or NH; and
enantiomers, diasteromers, tautomers, solvates, and pharmaceutically acceptable salts thereof.

Another embodiment of the present invention is a compound of Formula (I) wherein:
G is selected from —C(Z)N(R₁)R₂, 2-methylcarbonylaminophenyl, 2-aminocarbonyl-phenyl, 1H-tetrazol-4-yl, 2-methyl-tetrazol-5-yl, 4H-[1,2,4]-oxadiazol-5-oxo-3-yl, 4H-[1,2,4]-oxadiazol-5-thioxo-3-yl, 4H-[1,2,4]thiadiazol-5-oxo-3-yl, [1,2,3,5]oxathiadiazol-2-oxo-4-yl, or pyridin-3-yl;
R₁ is hydrogen, methyl, or ethyl;
R₂ is selected from the group consisting of hydrogen, C₁₋₄alkanyl and phenyl; wherein C₁₋₄alkanyl is optionally substituted with one to three substituents independently selected from the group consisting of phenyl, C₁₋₄alkanyloxy, hydroxy, fluoro, and phenoxy; and wherein any phenyl-containing substituent of R₂ is optionally substituted with one to three substituents independently selected from the group consisting of C₁₋₆alkanyl, C₁₋₆alkanyloxy, fluoro, and hydroxy;
or R₁ and R₂ taken together with the nitrogen to which they are attached form a pyrrolidinyl or piperidinyl ring;
R₃ is selected from the group consisting of hydrogen, C₁₋₈alkanyl, C₂₋₈alkenyl, C₂₋₈alkynyl, C₁₋₈alkanyloxy(C₁₋₈)alkanyl, C₁₋₈alkanylthio(C₁₋₈)alkanyl, hydroxyC₁₋₈alkanyl, thioformyl, phenylimino(C₁₋₈)alkanyl, phenyl(C₁₋₈)alkanyl, and heteroaryl(C₁₋₈)alkanyl wherein heteroaryl is selected from the group consisting of hydrogen, methyl, allyl, or heteroarylmethyl; wherein heteroaryl is selected from the group consisting of benzo[1,3]dioxolyl, imidazolyl, furanyl, pyridinyl, and thienyl; wherein phenyl and heteroaryl are optionally substituted with one to three substituents independently selected from the group consisting of C₁₋₆alkanyloxy and hydroxy; or optionally, when phenyl and heteroaryl are optionally substituted with two substituents attached to adjacent carbon atoms, the two substituents together form a single fused moiety; wherein the moiety is selected from —O(CH₂)₁₋₃O—;
R₄ is one to three substituents independently selected from the group consisting of hydrogen, C₁₋₄alkanyl, C₁₋₄alkanyloxy, halogen, phenyl, furanyl, imidazolyl, indazolyl, indolyl, indolinyl, isoindolinyl, isoquinolinyl, isothiazolyl, isoxazolyl, oxazolyl, pyrazinyl, pyrazolyl, pyridazinyl, pyridinyl, pyrimidinyl, pyrrolyl, quinolinyl, tetrazolyl, thiazolyl, thienyl, and hydroxy;
R₅ is hydrogen;
R₆ is one to two substituents independently selected from the group consisting of hydrogen and methyl;
Y is O or S;
Z is O or NH; and
   enantiomers, diastereomers, tautomers, solvates, and pharmaceutically acceptable salts thereof.

Another embodiment of the present invention is directed to compositions comprising a compound of Formula (I) wherein:
G is independently selected from —C(Z)N(R₁)R₂, 2-methylcarbonylaminophenyl, 2-aminocarbonyl-phenyl, 1H-tetrazol-4-yl, 2-methyl-tetrazol-5-yl, 4H-[1,2,4]-oxadiazol-5-oxo-3-yl, 4H-[1,2,4]-oxadiazol-5-thioxo-3-yl, 4H-[1,2,4]thiadiazol-5-oxo-3-yl, [1,2,3,5]oxathiadiazol-2-oxo-4-yl, and pyridin-3-yl;
R₁ is hydrogen, methyl, or ethyl;
R₂ is a substituent selected from the group consisting of hydrogen, C₁₋₄alkanyl and phenyl; wherein C₁₋₄alkanyl is optionally substituted with one to three substituents independently selected from the group consisting of phenyl, C₁₋₄alkanyloxy, hydroxy, and 2,6-dimethyl-phenoxy; and wherein the any phenyl-containing substituent of R₂ is optionally substituted with one to three substituents independently selected from the group consisting of C₁₋₆alkanyl, C₁₋₆alkanyloxy, fluoro, and hydroxy;
or R₁ and R₂ taken together with the nitrogen to which they are attached form a pyrrolidinyl or piperidinyl ring wherein said pyrrolidinyl or piperidinyl is optionally substituted with a substituent selected from the group consisting of C₁₋₃alkanyl and hydroxy;
R₃ is a substituent selected from the group consisting of benzo[1,3]dioxol-5-ylmethyl, carbamimidoyl, 1-H-imidazol-4-ylmethyl, phenyliminomethyl, 1-prop-2-ynyl, thioformyl, 2-hydroxyphenyl-methyl, hydroxy-ethyl, methoxy-ethyl, 2-methyl-allyl, 2-methyl-but-2-enyl, allyl, furan-3-ylmethyl, H, Me, methylthioethyl, phenethyl, pyridin-2-yl methyl, and thiophen-2-ylmethyl;
R₄ is one to two substituents independently selected from the group consisting of hydrogen, C₁₋₄alkanyl, C₁₋₄alkanyloxy, halogen, phenyl, furanyl, imidazolyl, indazolyl, indolyl, indolinyl, isoindolinyl, isoquinolinyl, isothiazolyl, isoxazolyl, oxazolyl, pyrazinyl, pyrazolyl, pyridazinyl, pyridinyl, pyrimidinyl, pyrrolyl, quinolinyl, tetrazolyl, thiazolyl, thienyl, and hydroxy;
R₅ is hydrogen;
R₆ is one to two substituents independently selected from the group consisting of hydrogen and methyl;
Y is O or S; and
Z is O or NH.

Another embodiment of the present invention is directed to compositions comprising a compound of Formula (I) wherein:
G is selected from —C(Z)N(R₁)R₂, 2-methylcarbonylaminophenyl, 2-aminocarbonyl-phenyl, 1H-tetrazol-4-yl, 2-methyl-tetrazol-5-yl, 4H-[1,2,4]-oxadiazol-5-oxo-3-yl, 4H-[1,2,4]-oxadiazol-5-thioxo-3-yl, 4H-[1,2,4]thiadiazol-5-oxo-3-yl, [1,2,3,5]oxathiadiazol-2-oxo-4-yl, or pyridin-3-yl;
R₁ is hydrogen, methyl, or ethyl;
R₂ is a substituent selected from the group consisting of hydrogen, C₁₋₄alkanyl and phenyl; wherein C₁₋₄alkanyl is optionally substituted with one to three substituents independently selected from the group consisting of phenyl, methoxy, hydroxy, and 2,6-dimethyl-phenoxy; and wherein any phenyl-containing substituent of R₂ is optionally substituted with one to three substituents independently selected from the group consisting of C₁₋₆alkanyl, C₁₋₆alkanyloxy, fluoro, and hydroxy;
or R₁ and R₂ taken together with the nitrogen to which they are attached form a pyrrolidinyl or piperidinyl ring wherein said pyrrolidinyl or piperidinyl are optionally substituted with a substituent selected from the group consisting of C₁₋₃alkanyl and hydroxy; R₃ is a substituent selected from the group consisting of benzo[1,3]dioxol-5-ylmethyl, carbamimidoyl, 1-H-imidazol-4-yl methyl, phenyliminomethyl, 1-prop-2-ynyl, thioformyl, 2-hydroxyphenyl-methyl, hydroxyethyl, methoxyethyl, allyl, furan-3-yl methyl, H, Me, methylthioethyl, and phenethyl;
R₄ is one to two substituents independently selected from the group consisting of hydrogen, methyl, methoxy, bromo, fluoro, α′- or β′-phenyl, α′- or β′-pyridinyl, α′- or β′-furanyl, and hydroxy:
R₅ is hydrogen;
R₆ is one to two substituents independently selected from the group consisting of hydrogen and methyl;
Y is O or S; and
Z is O or NH.

Another embodiment of the present invention is directed to compositions comprising a compound of Formula (I) wherein:
G is selected from —C(Z)N(R₁)R₂, 2-methylcarbonylaminophenyl, 2-aminocarbonyl-phenyl, 1H-tetrazol-4-yl, 2-methyl-tetrazol-5-yl, 4H-[1,2,4]-oxadiazol-5-oxo-3-yl, 4H-[1,2,4]-oxadiazol-5-thioxo-3-yl, 4H-[1,2,4]thiadiazol-5-oxo-3-yl, [1,2,3,5]oxathiadiazol-2-oxo-4-yl, or pyridin-3-yl;
R₁ is hydrogen, methyl, or ethyl;
R₂ is a substituent selected from the group consisting of hydrogen, C₁₋₄alkanyl and phenyl; wherein C₁₋₄alkanyl is optionally substituted with one to three substituents independently selected from the group consisting of phenyl, methoxy, hydroxy, and 2,6-dimethyl-phenoxy; and wherein any phenyl-containing substituent of R₂ is optionally substituted with one to three substituents independently selected from the group consisting of C₁₋₆alkanyl, C₁₋₆alkanyloxy, fluoro, and hydroxy;
alternatively R₁ and R₂ are taken together with the nitrogen to which they are attached to form a pyrrolidinyl or piperidinyl ring wherein said pyrrolidinyl or piperidinyl are optionally substituted with a substituent selected from the group consisting of C₁₋₃alkanyl and hydroxy;
R₃ is a substituent selected from the group consisting of H, benzo[1,3]dioxol-5-ylmethyl, 1-H-imidazol-4-yl methyl, furan-3-ylmethyl, pyridin-2-ylmethyl, and phenyliminomethyl;
R₄ is a substituent independently selected from the group consisting of hydrogen, methyl, methoxy, bromo, fluoro, α′- or β′-phenyl, α′- or β′-pyridinyl, α′- or β′-furanyl, and hydroxy;
R₅ is hydrogen;
R₆ is one to two substituents independently selected from the group consisting of hydrogen and methyl;
Y is O or S; and
Z is O or NH.

Another embodiment of the present invention is directed to compounds of Formula (J) and to compostions comprising compounds of Formula (I) wherein:
G is —C(Z)N(R₁)R₂, phenyl, or a heterocycle selected from the group consisting of tetrazolyl, oxadiazolyl, and pyridinyl; wherein phenyl and the heterocycles of G are optionally substituted with one to three substituents independently selected from the group consisting of C₁₋₄alkanylcarbonylamino and oxo;
R₁ is selected from the group consisting of hydrogen and ethyl;
R₂ is selected from the group consisting of hydrogen and C₁₋₄alkanyl;
or R₁ and R₂ taken together with the nitrogen to which they are attached form a pyrrolidinyl ring optionally substituted with hydroxy;
R₃ is hydrogen, C₁₋₈alkanyl, C₂₋₈alkenyl, C₃₋₈cycloalkanyl, phenyl(C₁₋₈)alkanyl, or heteroaryl(C₁₋₈)alkanyl wherein the heteroaryl is imidazolyl, furanyl, pyridinyl, or thienyl;
R₄ is one substituent selected from the group consisting of hydrogen, methoxy, chloro, and hydroxy;
R₅ is hydrogen;
R₆ is hydrogen;
Y is O or S; and
Z is O.

Another embodiment of the present invention is directed to compounds of Formula (J) and to compostions comprising compounds of Formula (I) wherein:
G is N,N-diethylaminocarbonyl, 3-(N,N-diethylaminocarbonyl)-phenyl, 2-methylcarbonylaminophenyl, N—N-diethylamidino, pyridin-3-yl, 3-(S)-hydroxypyrrolidin-1-ylcarbonyl, N-ethylaminocarbonyl, 1H-tetrazol-4-yl, pyridine-4-yl, or 4H-[1,2,4]-oxadiazol-5-oxo-3-yl;
R₁ is selected from the group consisting of hydrogen and ethyl;
R₂ is hydrogen or ethyl;
or R₁ and R₂ taken together with the nitrogen to which they are attached form a pyrrolidinyl ring optionally substituted with hydroxy;
R₃ is hydrogen, methyl, ethyl, propenyl, cyclopropylmethyl, benzyl, phenethyl, or heteroaryl wherein the heteroaryl is furanyl, imidazolyl, pyridinyl, or thienyl;
R₄ is one substituent and is hydrogen or hydroxy;
R₅ is hydrogen;
R₆ is hydrogen;
Y is O or S;
Z is O.

Still further embodiments of the invention relate to compounds of Formula (I) and to compositions containing one or more compounds of Formula (I) that are:
N-{2-[5-Hydroxy-9-(1-pyridin-2-ylmethyl-piperidin-4-yl)-9H-xanthen-3-yl]-phenyl}-acetamide;
N-{2-[5-Hydroxy-9-(1-thiophen-2-ylmethyl-piperidin-4-yl)-9H-xanthen-3-yl]-phenyl}-acetamide;
6-Pyridin-4-yl-9-(1-pyridin-2-ylmethyl-piperidin-4-yl)-9H-thioxanthen-4-ol;
N-{2-[5-Hydroxy-9-(1-pyridin-2-ylmethyl-piperidin-4-yl)-9H-thioxanthen-3-yl]-phenyl}-acetamide;
N-[2-(5-Hydroxy-9-piperidin-4-yl-9H-thioxanthen-3-yl)-phenyl]-acetamide;
9-Piperidin-4-yl-6-pyridin-3-yl-9H-thioxanthen-4-ol;
6-Pyridin-3-yl-9-(1-pyridin-2-ylmethyl-piperidin-4-yl)-9H-thioxanthen-4-ol;
9-Piperidin-4-yl-6-pyridin-4-yl-9H-thioxanthen-4-ol;
5-Hydroxy-9-piperidin-4-yl-9H-xanthene-3-carboxylic acid diethylamide;
9-Piperidin-4-yl-6-(1H-tetrazol-5-yl)-9H-xanthen-4-ol;
9-Piperidin-4-yl-9H-xanthene-3-carboxylic acid diethylamide;
N,N-Diethyl-9-(1-furan-3-ylmethyl-piperidin-4-yl)-9H-xanthene-3-carboxamidine;
9-(1-Allyl-piperidin-4-yl)-6-pyridin-3-yl-9H-xanthen-4-ol;
N,N-Diethyl-3-(5-hydroxy-9-piperidin-4-yl-9H-xanthen-3-yl)-benzamide;
N-[2-(5-Hydroxy-9-piperidin-4-yl-9H-xanthen-3-yl)-phenyl]-acetamide;
N,N-Diethyl-9-piperidin-4-yl-9H-xanthene-3-carboxamidine;
4-(5-Methoxy-9-piperidin-4-yl-9H-thioxanthen-3-yl)-pyridine;
9-(1-Benzyl-piperidin-4-yl)-9H-xanthene-3-carboxylic acid diethylamide;
3-(5-Methoxy-9-piperidin-4-yl-9H-thioxanthen-3-yl)-pyridine;
9-(1-Benzyl-piperidin-4-yl)-6-pyridin-3-yl-9H-xanthen-4-ol;
6-Pyridin-3-yl-9-(1-thiophen-2-ylmethyl-piperidin-4-yl)-9H-xanthen-4-ol;
N-{2-[9-(1-Furan-3-ylmethyl-piperidin-4-yl)-9H-xanthen-3-yl]-phenyl}-acetamide;
9-[1-(1H-Imidazol-2-ylmethyl)-piperidin-4-yl]-9H-xanthene-3-carboxylic acid diethylamide;
N-[2-(5-Methoxy-9-piperidin-4-yl-9H-thioxanthen-3-yl)-phenyl]-acetamide;
N-{2-[9-(1-Allyl-piperidin-4-yl)-5-hydroxy-9H-thioxanthen-3-yl]-phenyl}-acetamide;
9-(1-Furan-3-ylmethyl-piperidin-4-yl)-9H-xanthene-3-carboxylic acid diethylamide;
3-(9-Piperidin-4-yl-9H-xanthen-3-yl)-pyridine;
5-Hydroxy-9-piperidin-4-yl-9H-xanthene-3-carbonitrile;
N-{2-[9-(1-Pyridin-2-ylmethyl-piperidin-4-yl)-9H-xanthen-3-yl]-phenyl}-acetamide;
[9-(1-Furan-3-ylmethyl-piperidin-4-yl)-9H-xanthen-3-yl]-(3-(S)-hydroxy-pyrrolidin-1-yl)-methanone;
5-Hydroxy-9-piperidin-4-yl-9H-xanthene-3-carboxylic acid ethylamide;
9-Piperidin-4-yl-6-pyridin-3-yl-9H-xanthen-4-ol;
5-Chloro-9-piperidin-4-yl-9H-xanthene-3-carboxylic acid diethylamide;
5-Methoxy-9-piperidin-4-yl-9H-xanthene-3-carboxylic acid diethylamide;
3-[9-(1-Furan-3-ylmethyl-piperidin-4-yl)-9H-xanthen-3-yl]-pyridine;
3-(5-Methoxy-9-piperidin-4-yl-9H-xanthen-3-yl)-pyridine;
(3-(S)-Hydroxy-pyrrolidin-1-yl)-(9-piperidin-4-yl-9H-xanthen-3-yl)methanone;
9-(1-Thiophen-2-ylmethyl-piperidin-4-yl)-9H-xanthene-3-carboxylic acid diethylamide;
1-Furan-3-ylmethyl-4-[3-(1H-tetrazol-5-yl)-9H-xanthen-9-yl]-piperidine;
4-(5-Methoxy-9-piperidin-4-yl-9H-xanthen-3-yl)-pyridine;
3-(9-Piperidin-4-yl-9H-xanthen-3-yl)-4H-[1,2,4]oxadiazol-5-one;
N-[2-(5-Methoxy-9-piperidin-4-yl-9H-xanthen-3-yl)-phenyl]-acetamide;
4-[3-(1H-Tetrazol-5-yl)-9H-xanthen-9-yl]-piperidine;
3-[9-(1-Benzyl-piperidin-4-yl)-9H-xanthen-3-yl]-pyridine;
4-[9-(1-Furan-3-ylmethyl-piperidin-4-yl)-9H-xanthen-3-yl]-pyridine;
N-[2-(9-Piperidin-4-yl-9H-xanthen-3-yl)-phenyl]-acetamide;
9-(1-Cyclopropylmethyl-piperidin-4-yl)-9H-xanthene-3-carboxylic acid diethylamide;
N-{2-[9-(1-Allyl-piperidin-4-yl)-9H-xanthen-3-yl]-phenyl}-acetamide;
9-(1-Methyl-piperidin-4-yl)-9H-xanthene-3-carboxylic acid diethylamide;
4-[9-(1-Pyridin-2-ylmethyl-piperidin-4-yl)-9H-xanthen-3-yl]-pyridine;
9-(1-Pyridin-2-ylmethyl-piperidin-4-yl)-9H-xanthene-3-carboxylic acid diethylamide;
3-[9-(1-Phenethyl-piperidin-4-yl)-9H-xanthen-3-yl]-pyridine;
9-(1-Phenethyl-piperidin-4-yl)-9H-xanthene-3-carboxylic acid diethylamide;
4-(9-Piperidin-4-yl-9H-xanthen-3-yl)-pyridine;
N-{2-[9-(1-Phenethyl-piperidin-4-yl)-9H-xanthen-3-yl]-phenyl}-acetamide;
(3-(S)-Hydroxy-pyrrolidin-1-yl)-[9-(1-phenethyl-piperidin-4-yl)-9H-xanthen-3-yl]-methanone;
[9-(1-Allyl-piperidin-4-yl)-9H-xanthen-3-yl]-(3-(S)-hydroxy-pyrrolidin-1-yl)methanone;
3-{9-[1-(1H-Imidazol-2-ylmethyl)-piperidin-4-yl]-9H-xanthen-3-yl}-pyridine;
3-[9-(1-Thiophen-2-ylmethyl-piperidin-4-yl)-9H-xanthen-3-yl]-pyridine;
1-Phenethyl-4-[3-(1H-tetrazol-5-yl)-9H-xanthen-9-yl]-piperidine;
3-{9-[1-(1H-Imidazol-2-ylmethyl)-piperidin-4-yl]-9H-xanthen-3-yl}-4H-[1,2,4]oxadiazol-5-one;
9-Piperidin-4-yl-6-pyridin-4-yl-9H-xanthen-4-ol;
3-[9-(1-Furan-3-ylmethyl-piperidin-4-yl)-9H-xanthen-3-yl]-4H-[1,2,4]oxadiazol-5-one;
N,N-Diethyl-9-(1-phenethyl-piperidin-4-yl)-9H-xanthene-3-carboxamidine;
N-{2-[9-(1-Furan-3-ylmethyl-piperidin-4-yl)-5-hydroxy-9H-thioxanthen-3-yl]-phenyl}-acetamide;
N,N-Diethyl-5-hydroxy-9-piperidin-4-yl-9H-xanthene-3-carboxamidine; or
5-Hydroxy-9-(1-pyridin-2-ylmethyl-piperidin-4-yl)-9H-xanthene-3-carbonitrile.

Another embodiment of the present invention is directed to a compound of Formula (I) wherein R₄ is preferably substituted at the α′- or β′-position of Formula (I).

Another embodiment of the present invention is a composition comprising the dextrorotatory enantiomer of a compound of formula (I), wherein said composition is substantially free from the levorotatory isomer of said compound. In the present context, substantially free means less than 25%, preferably less than 10%, more preferably less than 5%, even more preferably less than 2% and even more preferably less than 1% of the levorotatory isomer calculated as.

$$\% levorotatory = \frac{( mass levorotatory )}{( mass dextrorotatory ) + ( mass levorotatory )} \times 100$$

Another embodiment of the present invention is a composition comprising the levorotatory enantiomer of a compound of formula (I) wherein said composition is substantially free from the dextrorotatory isomer of said compound. In the present context, substantially free from means less than 25%, preferably less than 10%, more preferably less than 5%, even more preferably less than 2% and even more preferably less than 1% of the dextrorotatory isomer calculated as

$$\% dextrorotatory = \frac{( mass dextrorotatory )}{( mass dextrorotatory ) + ( mass levorotatory )} \times 100$$

The compounds of the present invention may also be present in the form of pharmaceutically acceptable salts. For use in medicine, the salts of the compounds of this invention refer to non-toxic “pharmaceutically acceptable salts” (Ref. International J. Pharm., 1986, 33, 201-217; J. Pharm. Sci., 1997 (January), 66, 1, 1). Other salts well known to those in the art may, however, be useful in the preparation of compounds according to this invention or of their pharmaceutically acceptable salts. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc.

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-l-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

Even though the compounds of the present invention (including their pharmaceutically, acceptable salts and pharmaceutically acceptable solvates) can be administered alone, they will generally be administered in admixture with a pharmaceutical carrier, excipient or diluent selected with regard to the intended route of administration and standard pharmaceutical or veterinary practice. Thus, the present invention is directed to pharmaceutical and veterinary compositions comprising compounds of Formula (I) and one or more pharmaceutically acceptable carriers, excipients or diluents.

By way of example, in the pharmaceutical and veterinary compositions of the present invention, the compounds of the present invention may be admixed with any suitable binder(s), lubricant(s), suspending agent(s), coating agent(s), and/or solubilising agent(s).

Tablets or capsules of the compounds may be administered singly or two or more at a time, as appropriate. It is also possible to administer the compounds in sustained release formulations.

Alternatively, the compounds of the general Formula (I) can be administered by inhalation or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilizers and preservatives as may be required.

For some applications, preferably the compositions are administered orally in the form of tablets containing excipients such as starch or lactose, or in capsules or ovules either alone or in admixture with excipients, or in the form of elixirs, solutions or suspensions containing flavoring or coloring agents.

The compositions (as well as the compounds alone) can also be injected parenterally, for example intracavernosally, intravenously, intramuscularly or subcutaneously. In this case, the compositions will comprise a suitable carrier or diluent.

For parenteral administration, the compositions are best used in the form of a sterile aqueous solution which may contain other substances, for example enough salts or monosaccharides to make the solution isotonic with blood.

For buccal or sublingual administration the compositions may be administered in the form of tablets or lozenges which can be formulated in a conventional manner.

By way of further example, pharmaceutical and veterinary compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate the major site of absorption. For parenteral administration, the carrier will usually consist of sterile water and other ingredients may be added to increase solubility or preservation. Injectable suspensions or solutions may also be prepared utilizing aqueous carriers along with appropriate additives.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those skilled in that art. To be administered in the form of a transdermal delivery system, the dosage administration will, of course, be continuous rather than intermittent throughout the dosage regimen.

It is also apparent to one skilled in the art that the therapeutically effective dose for active compounds of the invention or a pharmaceutical composition thereof will vary according to the desired effect. Therefore, optimal dosages to be administered may be readily determined and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this invention.

Compounds of this invention may be administered in any of the foregoing compositions and dosage regimens or by means of those compositions and dosage regimens established in the art whenever use of the compounds of the invention as analgesics is required for a subject in need thereof.

The invention also provides a pharmaceutical or veterinary pack or kit comprising one or more containers filled with one or more of the ingredients of the pharmaceutical and veterinary compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The compounds of the present invention may be used to treat mild to severe pain in warm-blooded animals such as humans by administration of an analgesically effective dose. The dosage range would be from about 0.1 mg to about 15,000 mg, in particular from about 50 mg to about 3500 mg or, more particularly from about 100 mg to about 1000 mg of active ingredient in a regimen of about 1 to 4 times per day for an average (70 kg) human; although, it is apparent to one skilled in the art that the therapeutically effective amount for active compounds of the invention will vary as will the types of pain being treated.

For oral administration, a pharmaceutical composition is preferably provided in the form of tablets containing 0.01, 10.0, 50.0, 100, 150, 200, 250, and 500 milligrams of the active ingredient for the symptomatic adjustment of the dosage to the subject to be treated.

Examples of pain intended to be within the scope of the present invention include, but are not limited to, inflammatory pain, centrally mediated pain, peripherally mediated pain, visceral pain, structural or soft tissue injury related pain, progressive disease related pain, neuropathic pain and acute pain such as caused by acute injury, trauma or surgery and chronic pain such as headache and that caused by neuropathic conditions, post-stroke conditions, cancer, and migraine.

Compounds of the present invention are also useful as immunosuppressants, antiinflammatory agents, agents for the treatment and prevention of neurological and psychiatric conditions, for instance, depression and Parkinson's disease, agents for the treatment of urological and reproductive conditions, for instance, urinary incontinence and premature ejaculation, medicaments for drug and alcohol abuse, agents for treating gastritis and diarrhea, cardiovascular agents and cardioprotective agents and agents for the treatment of respiratory diseases.

The compounds of the present invention are also useful in treating pain caused by osteoarthritis, rheumatoid arthritis, fibromyalgia, migraine, headache, toothache, burn, sunburn, snake bite (in particular, venomous snake bite), spider bite, insect sting, neurogenic bladder, benign prostatic hypertrophy, interstitial cystitis, rhinitis, contact dermatitis/hypersensitivity, itch, eczema, pharyngitis, mucositis, enteritis, cellulites, causalgia, sciatic neuritis, mandibular joint neuralgia, peripheral neuritis, polyneuritis, stump pain, phantom limb pain, post-operative ileus, cholecystitis, postmastectomy pain syndrome, oral neuropathic pain, Charcot's pain, reflex sympathetic dystrophy, Guillain-Barre syndrome, meralgia paresthetica, burning-mouth syndrome, post-herpetic neuralgia, trigeminal neuralgia, cluster headache, migraine headache, peripheral neuropathy, bilateral peripheral neuropathy, diabetic neuropathy, postherpetic neuralgia, trigeminal neuralgia, optic neuritis, postfebrile neuritis, migrating neuritis, segmental neuritis, Gombault's neuritis, neuronitis, cervicobrachial neuralgia, cranial neuralgia, geniculate neuralgia, glossopharyngial neuralgia, migrainous neuralgia, idiopathic neuralgia, intercostals neuralgia, mammary neuralgia, Morton's neuralgia, nasociliary neuralgia, occipital neuralgia, red neuralgia, Sluder's neuralgia, splenopalatine neuralgia, supraorbital neuralgia, vidian neuralgia, inflammatory bowel disease, irritable bowel syndrome, sinus headache, tension headache, labor, childbirth, menstrual cramps, and cancer.

In regard to the use of the present compounds in treatment of the diseases or conditions such as those listed above, a therapeutically effective dose can be determined by persons skilled in the art by the use of established animal models. Such a dose would likely fall in the range of from about 0.01 mg to about 15,000 mg of active ingredient administered 1 to 4 times per day for an average (70 kg) human.

### General Synthetic Methods

Representative compounds of the present invention can be synthesized in accordance with the general synthetic methods described below and are illustrated in the schemes that follow. Since the schemes are an illustration, the invention should not be construed as being limited by the chemical reactions and conditions expressed. The preparation of the various starting materials used in the schemes is well within the skill of persons versed in the art.

The preparation of compounds of this invention is illustrated in Schemes 1 and 2. Both schemes proceed with the same overall strategy. In stage 1, an intermediates 1A and 1B is prepared with two benzene rings connected by a linker —Y—. The linker —Y— may be oxygen or sulfur. One benzene ring bears a group, Q, which is a group readily transformable to a substituent G as defined herein. Examples of such Q groups are fluoro, bromo, cyano, iodo, carboxy, or trifluoromethanesulfonyloxy. One benzene ring must bear a carboxylic acid, or a precursor to a carboxylic acid, positioned ortho to the linker —Y—. Schemes 1 and 2 differ in that in scheme 1, the carboxylic acid is on the benzene ring bearing the Q group (1A) while in scheme 2 the carboxylic acid function is on the benzene ring which does not bear the group Q (1B).

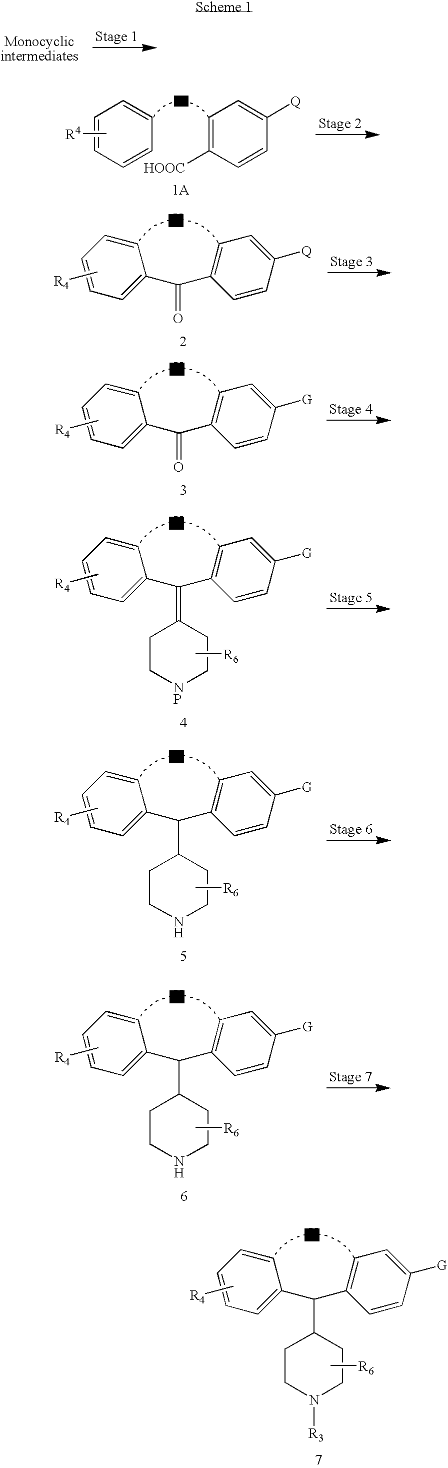

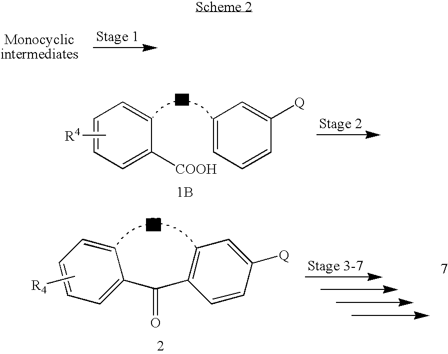

In stage 1 the linker —Y— is constructed between two monocyclic intermediates. For Scheme 1, Stage 1, the bridge may be constructed by nucleophilic aromatic displacement of fluoride from intermediate int 2 (where Q′ is an electron withdrawing group, readily convertible to a carboxylic acid, for instance cyano or alkoxycarbonyl) by a phenoxide or thiophenoxide, int 1. The 1A compounds are then obtained by hydrolysis of int 3 with an alkali metal hydroxide.

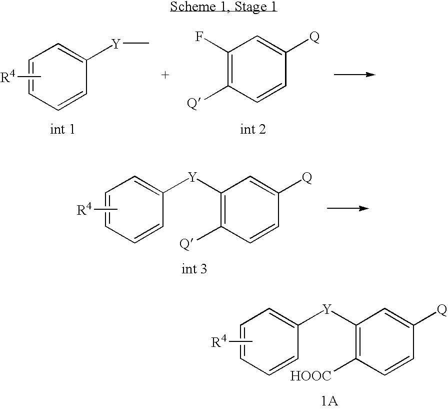

For Scheme 2, Stage 1, in order to prepare 1B compounds, the bridge may be constructed by nucleophilic aromatic displacement of fluoride from intermediate int 5 by phenoxides or thiophenoxides (int 4). The 1B compounds are then obtained by hydrolysis of int 6 with an alkali metal hydroxide.

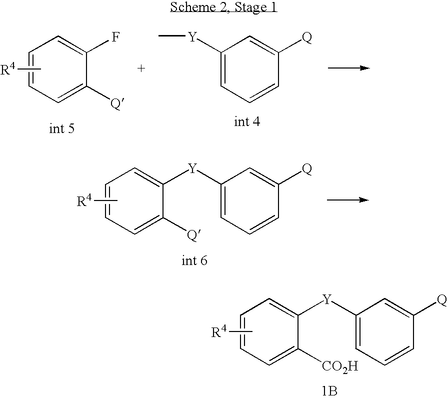

Following Stage 1, the schemes merge. In Stage 2, compounds 1A and 1B are converted by cycloacylation to ketones 2, using, for instance, BF₃.Et₂O-trifluoroacetic acid or polyphosphoric acid. Alternatively, the cyclization may be effected by converting acid 1A and 1B to an acid chloride, for instance with thionyl chloride, followed by Friedel-Crafts ring closure in the presence of a Lewis acid, such as aluminum chloride.

In addition, Stages 1 and 2 may be performed in reverse to give compounds 2 that are ready to enter Stage 3. For instance, Friedel-Crafts acylation between a methyl ether (int 7) and an appropriately substituted acid chloride (int 8) provides the ketone (int 9), which is simultaneously demethylated under the reaction conditions. Subsequent formation of the bridge —Y— via a nucleophilic aromatic displacement gives compounds 2 that are ready to enter Stage 3.

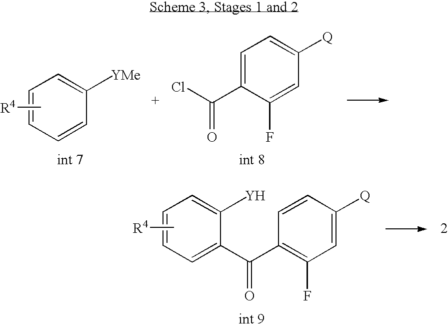

In stage 3, the Q function of compounds 2 is converted into group G, which may be —C(Z)NR₁R₂, an aryl substituent, or an appropriate heterocycle as defined herein, to give compounds of formula 3. When the Q function of compounds 2 is a halogen or trifluoromethanesulfonyloxy, it may be converted to an ester via alkoxycarbonylation using carbon monoxide, an aliphatic alcohol, a trialkanyl amine, and a palladium catalyst such as bis(triphenylphosphine) palladium(II) dichloride. Subsequently, when Q is an ester, the ester may be hydrolyzed to a carboxylic acid. The carboxylic acid may then be coupled with ammonia, a primary amine, or a secondary amine to form a primary, secondary or tertiary amide, respectively. Alternatively, the conversion of a carboxylic acid to an amide may be carried out via an acid chloride using thionyl chloride, oxalyl chloride, or the like, followed by a Schotten-Baumann reaction using ammonia or an amine in the presence of an alkali metal hydroxide. Alternatively, the conversion of a carboxylic acid to an amide may be carried out via the use of peptide coupling agents such as 1,3-dicyclohexylcarbondiimide (DCC), O-(7-azabenzotriazol-1-yl)-N,N,N′,N′-tetramethyluronium hexafluorophosphate (HATU), O-benzotriazol-1-yl-N,N,N′,N′-tetramethyluronium hexafluorophosphate (HBTU), or the like. Alternatively, the ester may be converted directly to the amide by the action of a dimethylaluminum amide.

Instead of proceeding to compounds 3 via an ester, one may effect the transformation of the group Q to a substituent G (wherein G is an amidino or heterocycle) by way of a nitrile. Synthesis of the nitrile may be accomplished by treatment of the compounds 2 (when Q is bromo or trifluoromethanesulfonyloxy) with Zn(CN)₂ and a palladium catalyst such as (Ph₃P)₄Pd or by treatment of the compounds 2 with CuCN at elevated temperatures. For the synthesis of amidino functional groups, the nitrile is treated with hydroxylamine under basic conditions to afford an oxime. Treatment of the oxime with a primary or secondary amine, CuCl, and an alkali metal carbonate under microwave irradiation in an alcoholic solvent provides the amidino compounds of the present invention. Microwave accelerated reactions may be performed using either a CEM Discover or a Personal Chemistry Smith Synthesizer microwave instrument. The oxime described above is instrumental in the preparation of compounds wherein G is a heterocycle. The oxime may be cyclized with a variety of electrophiles known to one versed in the art to give the heterocycles of the present invention. For instance, reaction of an oxime with CDI provides oxadiazolones, and treatment of the oxime with TCDI provides the corresponding oxadiazolethiones. Similarly, the treatment of the oxime with thionyl chloride in the presence of a tertiary amine gives oxathiadiazoles of the present invention.

Alternatively, compounds where Q is a halogen atom or a trifluoromethanesulfonyloxy group may participate in transition metal-mediated coupling reactions such as Suzuki, Stille or Negishi chemistry.

To perform stage 4, an appropriately substituted 4-piperidinylidene function is attached to the tricyclic system, replacing the ketone to give compounds of type 4. This operation may be carried out by McMurray condensation of ketones 3 with an appropriately substituted 4-piperidone species brought about by a lower valent titanium reagent such as the reagent obtained from addition of titanium tetrachloride to zinc dust. Alternatively, an appropriately substituted 4-piperidinyl magnesium halide may be added to ketone to afford carbinols. Dehydration of such carbinols with acidic reagents such as formic acid, sulfuric acid or trifluoroacetic acid gives rise to compounds of type 4.

If desired, the operation of stages 3 and 4 may be carried out in reverse order.

As illustrated in Schemes 1 and 2, the nitrogen atoms of compounds 4 may bear a group P. This group may be an alkanyl, alkenyl or aralkanyl in which case they are the therapeutically useful products of this invention. The group P may also be trifluoromethylcarbonyl, alkoxycarbonyl or aralkoxycarbonyl.

The olefin in compound 4 may be reduced to obtain the corresponding alkane (stage 5). This transformation may be carried out by treatment of compounds 4 with hydrogen iodide in chloroform or a mixture of trimethylsilyl iodide and ethanol in chloroform to yield compounds 5. The group P can be removed to produce free amines 6 (stage 6). This transformation may be carried out using certain acidic reagents such as hydrogen bromide or trimethylsilyl iodide. Or, when P is a trifluoromethylcarbonyl, basic reagents such as potassium carbonate in an alcoholic solvent may be used for the removal of P. Compounds of type 5 bearing readily cleavable groups such as methyl, allyl or benzyl may be transformed into the aforementioned alkoxycarbonyl derivatives by treatment with alkanylchloroformates such as ethyl chloroformate or 1-chloroethyl chloroformate.

Stages 5 and 6 may be performed in reverse to give compounds 6. In this case, group P is removed as described above before the olefin is reduced.

Finally, the secondary amines 6 may be converted to any desired end product of the invention 7 as shown in Stage 7. These transformations may be carried out by reductive alkylation using a carbonyl compound and a reducing agent such as sodium borohydride, sodium cyanoborohydride, sodium triacetoxyborohydride, or tetramethylammonium triacetoxyborohydride. They may also be carried out by alkylation using an alkanyl, alkenyl or aralkyl halide and an organic or inorganic base.

Finally, the transformation of compound 4 into compound 7 may also be performed by performing stages 5 through 7 in the following order: stage 6, followed by stage 7, followed by stage 5. In this case, group P is removed first. In the second step, R₃ is introduced as described above, and the final step consists of reduction of the olefin to the corresponding saturated carbon-carbon bond.

Desired end products of the present invention may include chemical modifications at R₄. Such transformations may include the dealkylation of lower alkyl ethers to give their corresponding alcohols, using reagents such as boron trihalides. Compounds where R₄ is a halogen atom may participate in transition metal-mediated coupling reactions such as Suzuki, Stille or Negishi chemistry.

The compounds wherein the two phenyl rings are substituted in a non-symmetrical fashion are chiral. They may be separated into their enantiomers by chromatography on a chiral stationary phase following Stages 4, 5, or 6. Alternatively, the basic compounds of types 5, 6, and 7 may be converted to diastereomeric salts by mixture with a chiral acid and resolved into their enantiomers by fractional crystallization.

It is generally preferred that the respective product of each process step be separated from other components of the reaction mixture and subjected to purification before its use as a starting material in a subsequent step. Separation techniques typically include evaporation, extraction, precipitation and filtration. Purification techniques typically include column chromatography (Still, W. C. et. al., J. Org. Chem. 1978, 43, 2921), thin-layer chromatography, crystallization and distillation. The structures of the final products, intermediates and starting materials are confirmed by spectroscopic, spectrometric and analytical methods including nuclear magnetic resonance (NMR), mass spectrometry (MS) and liquid chromatography (HPLC). In the descriptions for the preparation of compounds of this invention, ethyl ether, tetrahydrofuran and dioxane are common examples of an ethereal solvent; benzene, toluene, hexanes and heptanes are typical hydrocarbon solvents and dichloromethane and dichloroethane are representative halogenated hydrocarbon solvents. In those cases where the product is isolated as the acid addition salt the free base may be obtained by techniques known to those skilled in the art. In those cases in which the product is isolated as an acid addition salt, the salt may contain one or more equivalents of the acid. Enantiomers of the compounds of the present invention may be separated using chiral HPLC.

Representative compounds of the present invention can be synthesized in accordance with the general synthetic methods described above and are illustrated more particularly in the schemes that follow. Since the schemes are illustrations, the invention should not be construed as being limited by the chemical reactions and conditions expressed. The preparation of the various starting materials used in the schemes is well within the skill of persons versed in the art.

### Abbreviations

Boc=tert-butoxycarbonyl
DMF=N,N-dimethylformamide
Et=ethyl
h=hour(s)
Me=methyl
min=minute(s)
TFA=trifluoroacetic acid
THF=Tetrahydrofuran
TMSI=trimethylsilyl iodide

### EXAMPLES

### Example A

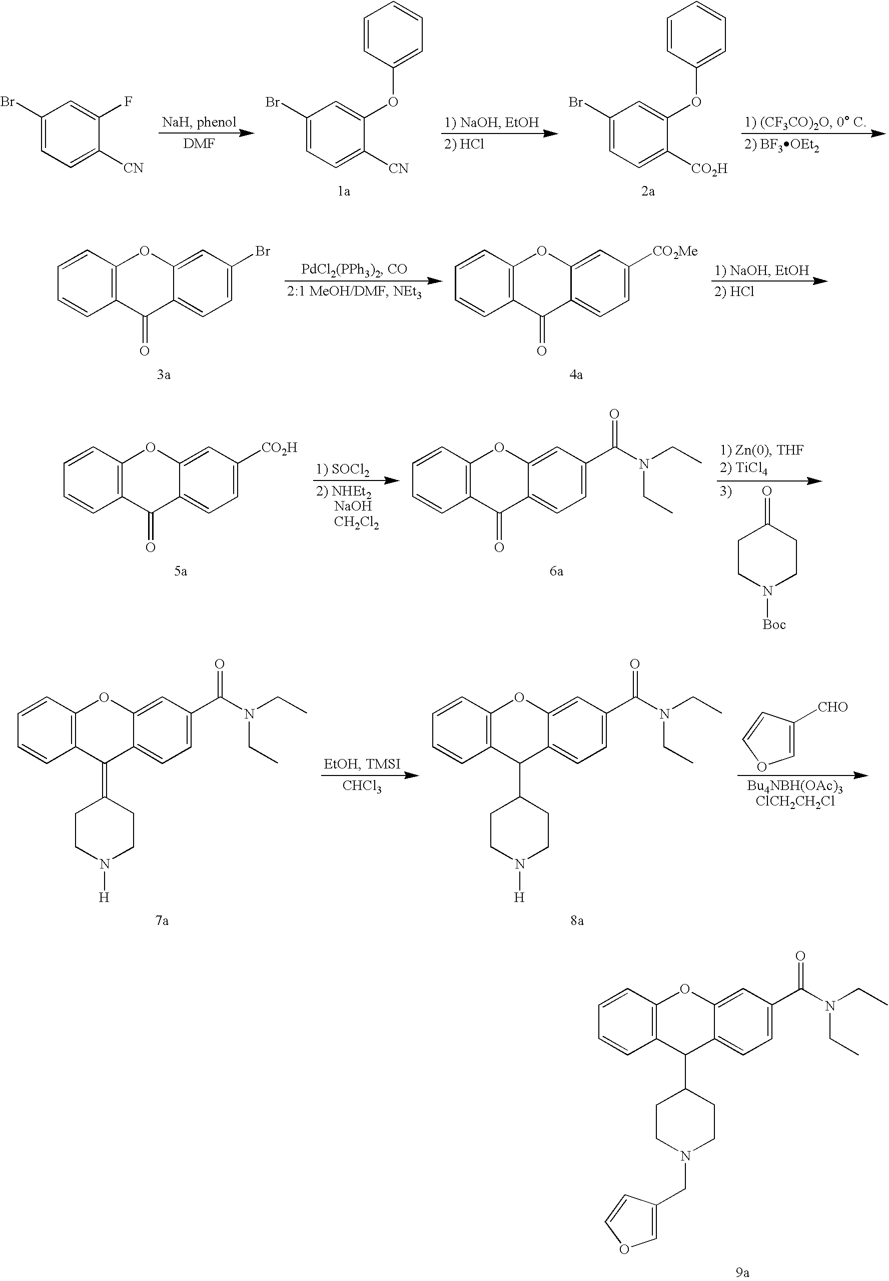

### Procedure 1

### 4-Bromo-2-phenoxybenzonitrile, 1a

Sodium hydride (12 g, 300 mmol) (60% by wt) was weighed into a flask and washed free of oil with several hexane rinsings. The hexanes were decanted and discarded and DMF was added to the flask. A DMF solution of phenol (23.5 g, 250 mmol in 100 mL DMF) was added dropwise to the NaH mixture and stirred at room temperature. To the phenoxide was added dropwise a solution of 4-bromo-2-fluorobenzonitrile (50 g, 250 mmol in 100 mL DMF). Upon complete addition, the reaction was refluxed for 20 h. The reaction was cooled to room temperature, and poured into cold 1 N NaOH. A fine, tan precipitate formed and was collected by vacuum filtration to give 62.04 g (226 mmol) of 4-bromo-2-phenoxybenzonitrile, 1a. MS m/z (MH⁺) 277.

### Procedure 2

### 4-Bromo-2-phenoxybenzoic acid, 2a

4-Bromo-2-phenoxybenzonitrile (35.3 g, 129 mmol) was added to 130 mL EtOH, followed by the addition of 340 mL of 20% NaOH (aq). The reaction was heated to reflux for 20 h. The mixture was cooled to room temperature and poured into 6 N HCl and a precipitate formed. The solid was collected by vacuum filtration and dissolved in 3:1 THF-ethyl ether and washed with brine. The organic phase was dried over magnesium sulfate, and concentrated. The solids were dried in a vacuum oven at 60° C. overnight to give 35.1 g (128 mmol) of title compound 4-bromo-2-phenoxybenzoic acid, 2a. MS m/z (MH⁺) 292.

### Procedure 3

### 3-Bromoxanthen-9-one, 3a

To a suspension of 4-bromo-2-phenoxybenzoic acid (35.1 g, 120 mmol) in methylene chloride (350 mL) at 0° C. was added dropwise trifluoroacetic anhydride (20.3 mL, 144 mmol), and the reaction was stirred for 15 min. At that time, boron trifluoride diethyl etherate (1.46 mL, 12.0 mmol) was added dropwise. The reaction became homogeneous upon stirring for 1 h at room temperature. Upon completion, the reaction was poured into 1 N NaOH, and the organic phase was dried over magnesium sulfate, filtered, and concentrated to give title compound 3-bromoxanthen-9-one, 3a (32.14 g, 116 mmol). MS m/z (MH⁺) 275.

### Procedure 4

### 9-Oxo-9H-xanthene-3-carboxylic acid methyl ester, 4a

A sample of 3-bromoxanthen-9-one (20 g, 72.2 mmol) was dissolved in a 2:1 MeOH/DMF solution (600 mL). To this solution was added triethylamine (40 mL, 290 mmol) and the solution was degassed with Argon. To this was added dichlorobis(triphenylphosphine) palladium (II) (2.0 g, 2.85 mmol), and the reaction was transferred to a bomb and charged with 150 psi of CO (g). The reaction was heated at 90° C. for 24 h. Upon completion, the reaction was cooled to 40° C. and methylene chloride was added. The reaction was filtered while warm and evaporated to provide the crude product. Recrystallization from ethanol gave 16.62 g (65.4 mmol) of title compound 9-oxo-9H-xanthene-3-carboxylic acid methyl ester, 4a. MS m/z (MH⁺) 255.

### Procedure 5

### 9-Oxo-9H-xanthene-3-carboxylic acid, 5a

A sample of 9-oxo-9H-xanthene-3-carboxylic acid methyl ester, 4a (16.6 g, 65.3 mmol) was suspended in 250 mL of 3 N NaOH and 250 mL of EtOH and heated to reflux for 1 h. At that time the EtOH was evaporated and the reaction was poured into 6 N HCl over ice and extracted with large volumes of 1:1 THF/diethyl ether. The combined organic phases were washed with brine, dried over magnesium sulfate, filtered and evaporated to provide 13.35 g of title compound 9-oxo-9H-xanthene-3-carboxylic acid, 5a (55.6 mmol) after drying in a vacuum oven at 50° C. overnight.

### Procedure 6

### 9-Oxo-9H-xanthene-3-carboxylic acid diethylamide, 6a

A sample of 9-oxo-9H-xanthene-3-carboxylic acid (13.4 g, 55.6 mmol) was suspended in methylene chloride (220 mL) and thionyl chloride (24.4 mL, 330 mmol) was added. The mixture was refluxed for 6 h, adding approximately 10 mL of additional thionyl chloride per hour until the reaction became homogeneous. At that time, the thionyl chloride and solvent were removed under vacuum and the remaining residue was diluted with an additional 220 mL methylene chloride. To the suspension was added 100 mL ice cold 1.5 N NaOH, 100 mL methylene chloride, and (17 mL, 166 mmol) diethyl amine. After stirring for 15 min at room temperature, the organic phase was separated and washed with HCl and brine, dried over magnesium sulfate, filtered and concentrated to yield title compound 9-oxo-9H-xanthene-3-carboxylic acid diethylamide, 6a (14.7 g, 49.8 mmol). MS m/z (MH⁺) 296.

### Procedure 7

### 9-Piperidin-4-ylidene-9H-xanthene-3-carboxylic acid diethylamide, 7a

To a suspension of zinc metal dust (1.83 g; 28 mmol) in THF (25 mL) under Argon was added titanium (IV) tetrachloride (1.55 mL; 14.1 mmol). The mixture was refluxed for 2 hr. After cooling of the mixture to rt, a solution of 9-oxo-9H-xanthene-3-carboxylic acid diethylamide, 6a (1.04 g; 3.5 mmol), and 4-oxopiperidine-1-carboxylic acid tert-butyl ester (0.7 g; 3.5 mmol) in THF (0.1 to 1.0 M solution) were added. The mixture was refluxed for 2 hr. The mixture was allowed to cool to rt, poured into excess potassium carbonate in ice water and extracted with ethyl acetate. The organic layer was separated, washed with brine, dried over potassium carbonate, filtered, and evaporated to yield 1.28 g (quant.) of title compound 9-piperidin-4-ylidene-9H-xanthene-3-carboxylic acid diethylamide, 7a.

### Procedure 8

### 9-Piperidin-4-yl-9H-xanthene-3-carboxylic acid diethylamide, 8a

To a solution of 9-piperidin-4-ylidene-9H-xanthene-3-carboxylic acid diethylamide, 7a (2.87 g, 7.92 mmol) in chloroform (50 mL) was added ethanol (3.23 mL, 55.4 mmol) and trimethylsilyl iodide (5.4 mL, 39.6 mmol) and the mixture was stirred at 100° C. for 1 to 4 h in a sealed tube. The reaction was allowed to cool to rt and washed with 1 N NaOH, aqueous Na₂S₂O₄, and brine. The organic phase can dried over sodium sulfate, filtered, and concentrated, to yield 1.66 g (57.7%) of title compound 9-piperidin-4-yl-9H-xanthene-3-carboxylic acid diethylamide, 8a. MS m/z (MH⁺) 365.1.

### Procedure 9

### 9-(1-Furan-3-ylmethyl-piperidin-4-yl)-9H-xanthene-3-carboxylic acid diethylamide, 9a

To a solution of 9-piperidin-4-yl-9H-xanthene-3-carboxylic acid diethylamide, 8a (153 mg, 0.42 mmol) in dichloroethane (4 mL) was added tetrabutylammonium triacetoxyborohydride (220 mg, 0.83 mmol), N,N-diisopropyl-N-ethylamine (73 μL, 0.42 mmol and 3-furaldehyde (109 μL, 1.2 mmol). The reaction was stirred at rt for 18 h. The mixture was washed with 1 N NaOH and brine, and the organic phase was separated and dried over sodium sulfate. After filtration and evaporation, the residue was purified via reverse phase column chromatography (eluent: CH₃CN in H₂O containing 0.1% TFA) to yield 141 mg (60%) of title compound 9-(1-furan-3-ylmethyl-piperidin-4-yl)-9H-xanthene-3-carboxylic acid diethylamide, 9a as a TFA salt. MS m/z (MH⁺) 445.1.

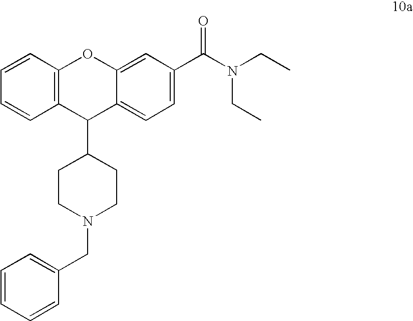

### 9-(1-Benzyl-piperidin-4-yl)-9H-xanthene-3-carboxylic acid diethylamide, 10a

Using an adaptation of the method described in Procedure 9, substituting benzaldehyde for 3-furaldehyde, the title compound 9-(1-benzyl-piperidin-4-yl)-9H-xanthene-3-carboxylic acid diethylamide, 10a was obtained as a TFA salt. MS m/z (MH⁺) 455.1.

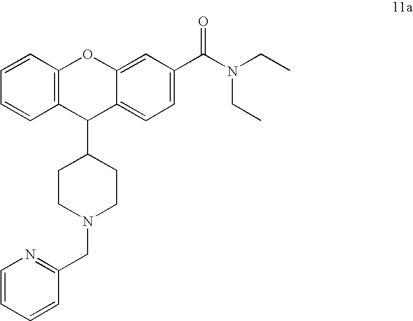

### 9-(1-Pyridin-2-ylmethyl-piperidin-4-yl)-9H-xanthene-3-carboxylic acid diethylamide, 11a

Using an adaptation of the method described in Procedure 9, substituting 2-pyridyl carboxaldehyde for 3-furaldehyde, the title compound 9-(1-pyridin-2-ylmethyl-piperidin-4-yl)-9H-xanthene-3-carboxylic acid diethylamide, 11a was obtained as a TFA salt. MS m/z (MH⁺) 456.1.

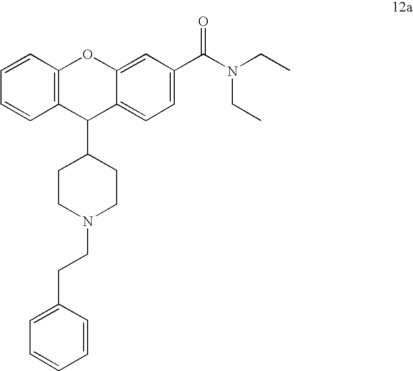

### 9-(1-Phenethyl-piperidin-4-yl)-9H-xanthene-3-carboxylic acid diethylamide, 12a

Using an adaptation of the method described in Procedure 9, substituting phenylacetaldehyde for 3-furaldehyde, the title compound 9-(1-phenethyl-piperidin-4-yl)-9H-xanthene-3-carboxylic acid diethylamide, 12a was obtained as a TFA salt. MS m/z (MH⁺) 469.2.

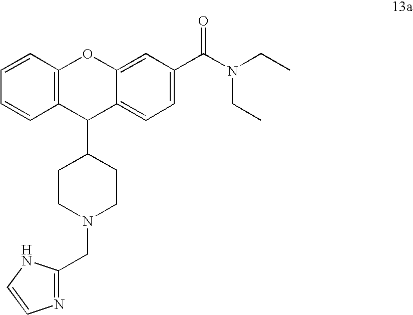

### 9-[1-(1H-Imidazol-2-ylmethyl)-piperidin-4-yl]-9H-xanthene-3-carboxylic acid diethylamide, 13a

Using an adaptation of the method described in Procedure 9, substituting 1H-imidazole-2-carboxaldehyde for 3-furaldehyde, the title compound 9-[1-(1H-imidazol-2-ylmethyl)-piperidin-4-yl]-9H-xanthene-3-carboxylic acid diethylamide, 13a was obtained as a TFA salt. MS m/z (MH⁺) 445.1.

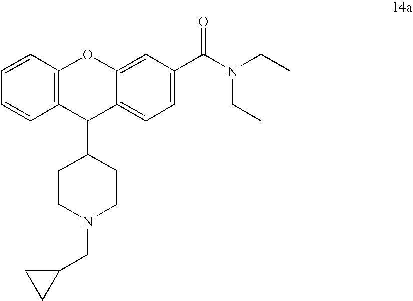

### 9-(1-Cyclopropylmethyl-piperidin-4-yl)-9H-xanthene-3-carboxylic acid diethylamide, 14a

Using an adaptation of the method described in Procedure 9, substituting cyclopropylcarboxaldehyde for 3-furaldehyde, the title compound 9-(1-cyclopropylmethyl-piperidin-4-yl)-9H-xanthene-3-carboxylic acid diethylamide, 14a was obtained as a TFA salt. MS m/z (MH⁺) 419.1.

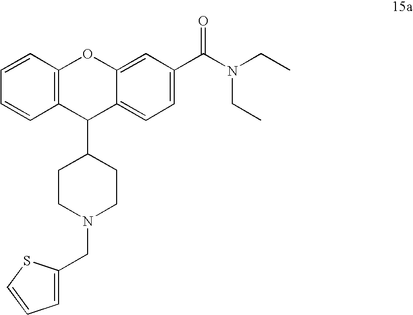

### 9-(1-Thiophen-2-ylmethyl-piperidin-4-yl)-9H-xanthene-3-carboxylic acid diethylamide, 15a

Using an adaptation of the method described in Procedure 9, substituting 2-thiophenecarboxaldehyde for 3-furaldehyde, the title compound 9-(1-thiophen-2-ylmethyl-piperidin-4-yl)-9H-xanthene-3-carboxylic acid diethylamide, 15a was obtained as a TFA salt. MS m/z (MH⁺) 461.1.

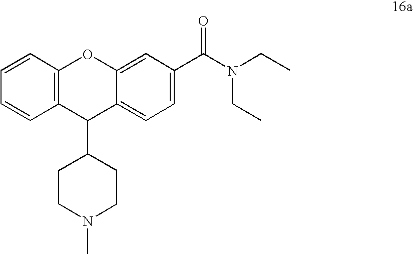

### 9-(1-Methyl-piperidin-4-yl)-9H-xanthene-3-carboxylic acid diethylamide, 16a

Using an adaptation of the method described in Procedure 9, substituting paraformaldehyde for 3-furaldehyde, the title compound 9-(1-methyl-piperidin-4-yl)-9H-xanthene-3-carboxylic acid diethylamide, 16a was obtained as a TFA salt. MS m/z (MH⁺) 379.1.

### Example B

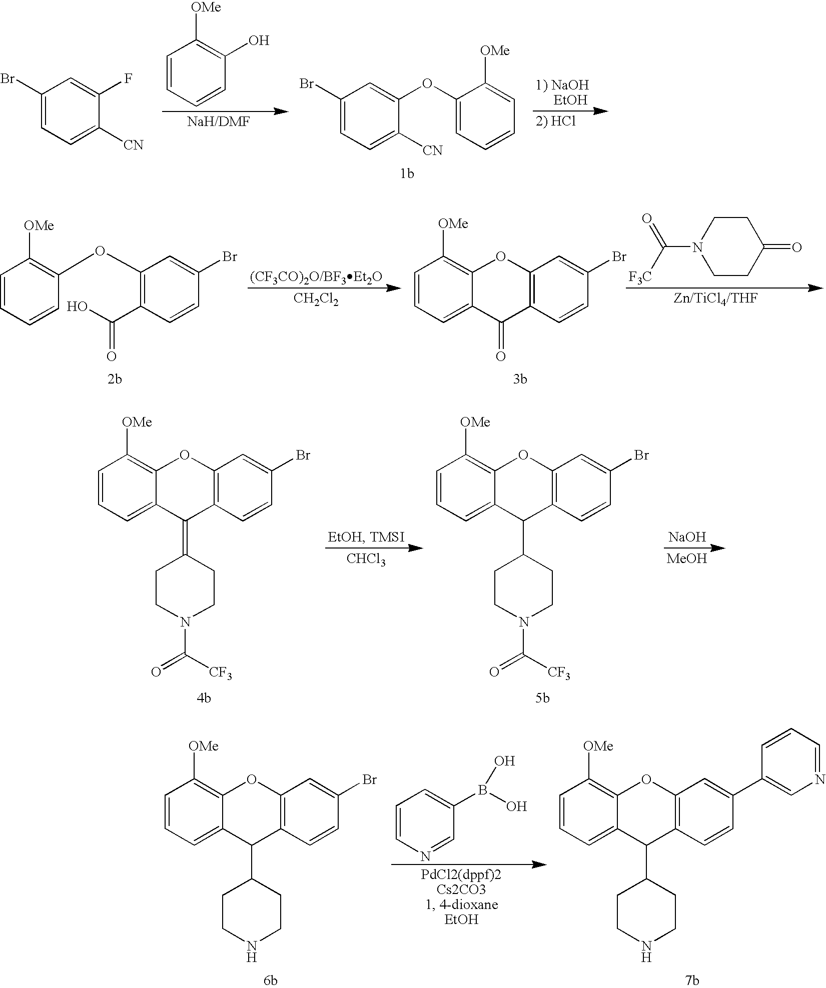

### 4-Bromo-2-(2-methoxy-phenoxy)-benzonitrile, 1b

Using an adaptation of the method described in Procedure 1, substituting 2-methoxyphenol for phenol, the title compound 4-bromo-2-(2-methoxyphenoxy)-benzonitrile 1b was prepared.

### 4-Bromo-2-(2-methoxy-phenoxy)-benzoic acid, 2b

Using an adaptation of the method described in Procedure 2, substituting Compound 1b for Compound 1a, the title compound 4-bromo-2-(2-methoxyphenoxy)-benzoic acid 2b was prepared.

### 3-Bromo-5-methoxy-xanthen-9-one, 3b

Using an adaptation of the method described in Procedure 3, substituting Compound 2b for Compound 2a, the title compound 3-bromo-5-methoxy-xanthen-9-one 3b was prepared.

### 1-[4-(3-Bromo-5-methoxy-xanthen-9-ylidene)-piperidin-1-yl]-2,2,2-trifluoro-ethanone, 4b

Using an adaptation of the method described in Procedure 7, substituting Compound 3b for Compound 6a and substituting 1-(2,2,2-trifluoroacetyl)piperidin-4-one for 3-oxo-8-aza-bicyclo[3.2.1]-octane-8-carboxylic acid tert-butyl ester, the title compound 1-[4-(3-bromo-5-methoxy-xanthen-9-ylidene)piperidin-1-yl]-2,2,2-trifluoro-ethanone, 4b was prepared. MS m/z (MH⁺) 467.9.

### 1-[4-(3-Bromo-5-methoxy-9H-xanthen-9-yl)-piperidin-1-yl]-2,2,2-trifluoro-ethanone, 5b

Using an adaptation of the method described in Procedure 8, substituting 1-[4-(3-bromo-5-methoxy-xanthen-9-ylidene)-piperidin-1-yl]-2,2,2-trifluoro-ethanone, 4b for 9-piperidin-4-ylidene-9H-xanthene-3-carboxylic acid diethylamide, 8a and, the title compound 1-[4-(3-bromo-5-methoxy-9H-xanthen-9-yl)-piperidin-1-yl]-2,2,2-trifluoro-ethanone, 5b was prepared. MS m/z (MH⁺) 470.1/471.8.

### Procedure 10

### 4-(3-Bromo-5-methoxy-9H-xanthen-9-yl)-piperidine, 6b

To a solution of 1-[4-(3-bromo-5-methoxy-9H-xanthen-9-yl)-piperidin-1-yl]-2,2,2-trifluoro-ethanone, 5b (3.5 g, 7.46 mmol) in methanol (25 mL) was added a 3N NaOH solution (3 mL). The mixture was heated to reflux for 1 h. The mixture was allowed to cool to rt, methylene chloride was added, and the organic phase was separated, dried, filtered, and evaporated to yield 3.0 g (quant.) of title compound 4-(3-bromo-5-methoxy-9H-xanthen-9-yl)-piperidine, 6b. MS m/z (MH⁺) 373.9.

### Procedure 11

### 3-(5-Methoxy-9-piperidin-4-yl-9H-xanthen-3-yl)-pyridine, 7b

A mixture of 4-(3-bromo-5-methoxy-9H-xanthen-9-yl)-piperidine, 6b (0.3 g, 0.8 mmol), 3-pyridyl boronic acid (0.15 g, 1.2 mmol), and PdCl₂(dppf)₂ (0.031 g, 0.04 mmol) in a sat Na₂CO₃ solution (1 mL) was heated to 60° C. for 4 h. The mixture was allowed to cool to rt, filtered, and evaporated. The mixture was poured onto ice and extracted with EtOAc. The organic phase was dried over K₂CO₃, filtered, and evaporated. The residue was purified via reverse phase HPLC (eluent: acetonitrile in water containing 0.1% TFA) to yield 2.4 mg (0.6%) title compound 3-(5-methoxy-9-piperidin-4-yl-9H-xanthen-3-yl)-pyridine, 7b was obtained as a TFA salt. MS m/z (MH⁺) 373.1.

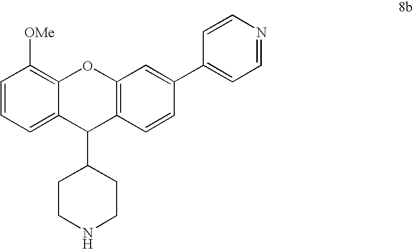

### 4-(5-Methoxy-9-piperidin-4-yl-9H-xanthen-3-yl)-pyridine, 8b

Using an adaptation of the method described in Procedure 11, substituting 4-pyridyl boronic acid for 3-pyridyl boronic acid, the title compound 4-(5-methoxy-9-piperidin-4-yl-9H-xanthen-3-yl)-pyridine, 8b was obtained as a TFA salt. MS m/z (MH⁺) 372.9.

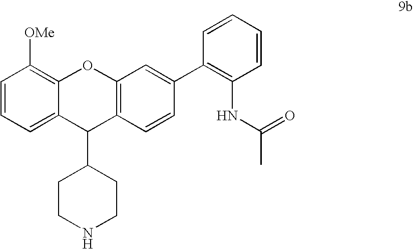

### N-[2-(5-Methoxy-9-piperidin-4-yl-9H-xanthen-3-yl)-phenyl]-acetamide, 9b

Using an adaptation of the method described in Procedure 11, substituting 2-acetylaminophenyl boronic acid for 3-pyridyl boronic acid, the title compound N-[2-(5-methoxy-9-piperidin-4-yl-9H-xanthen-3-yl)-phenyl]-acetamide, 9b was obtained as a TFA salt. MS m/z (MH⁺) 429.2.

### Example C

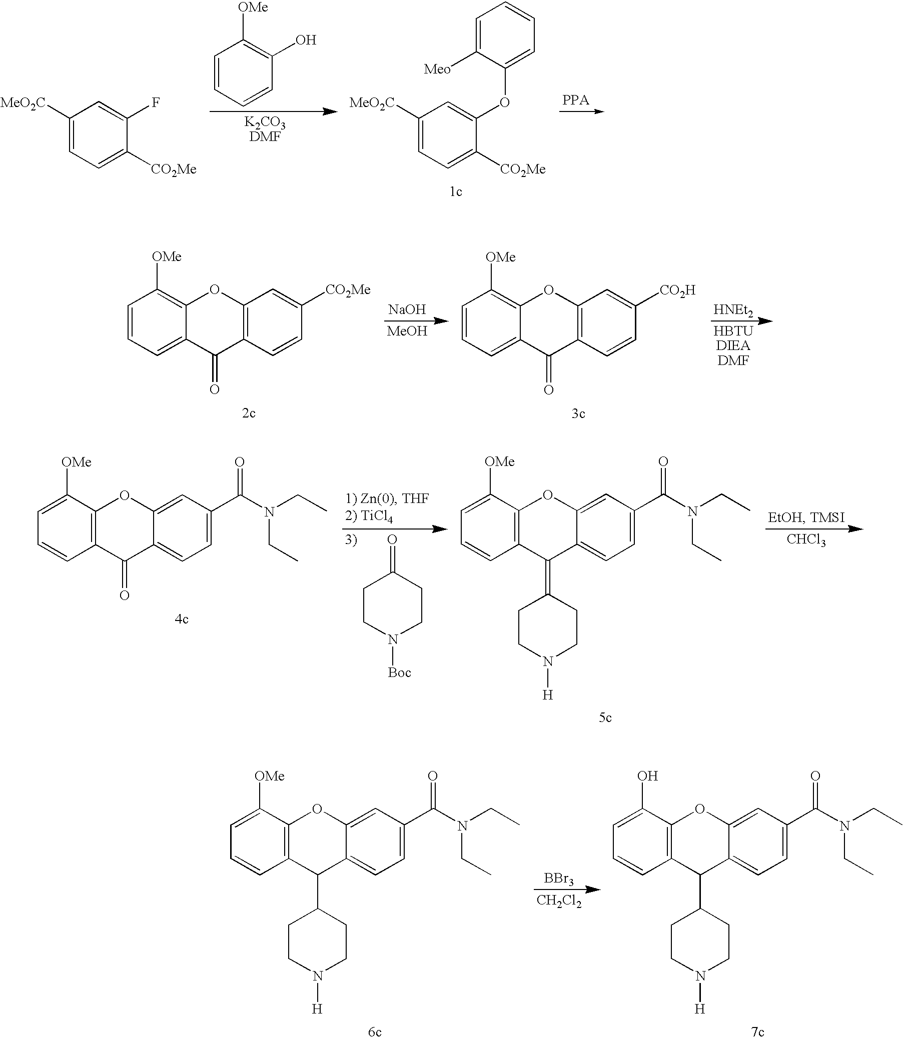

### 2-(2-Methoxy-phenoxy)-terephthalic acid dimethyl ester, 1c

Using an adaptation of the method described in Procedure 1, substituting 2-methoxyphenol for phenol and potassium carbonate for sodium hydride, the title compound 2-(2-methoxyphenoxy)-terephthalic acid dimethyl ester, 1c was prepared.

### Procedure 12

### 5-Methoxy-9-oxo-9H-xanthene-3-carboxylic acid methyl ester, 2c

A solution of 2-(2-methoxyphenoxy)-terephthalic acid dimethyl ester, 1c (12.8 g, 40.5 mmol) in polyphosphoric acid (290 g) was heated at 125° C. while being agitated with a mechanical stirrer. The mixture was poured into ice-water and stirred overnight. The solid was separated via filtration, washed with water, and air-dried. Flash column chromatography over silica gel (eluent mixture of MeOH in CH₂Cl₂) yielded 6.48 g (56.3%) of 5-methoxy-9-oxo-9H-xanthene-3-carboxylic acid methyl ester, 2c.

### Procedure 13

### 5-Methoxy-9-oxo-9H-xanthene-3-carboxylic acid, 3c

To a solution of 5-methoxy-9-oxo-9H-xanthene-3-carboxylic acid methyl ester, 2c (6.48 g, 22.8 mmol) in methanol (100 mL) was added 3N NaOH (10 mL) and the mixture was heated to reflux for 3 hr. The mixture was evaporated, dissolved in water, and acidified with conc. HCl. The solid was separated via filtration, washed with water, and air-dried to yield 5.6 g (quant.) of 5-methoxy-9-oxo-9H-xanthene-3-carboxylic acid, 3c.

### Procedure 14

### 5-Methoxy-9-oxo-9H-xanthene-3-carboxylic acid diethylamide, 4c

To a solution of 5-methoxy-9-oxo-9H-xanthene-3-carboxylic acid 3c (6.8 g, 25.2 mmol) and HBTU (10.0 g; 26.4 mmol) in DMF (70 mL) was added N,N-diisopropyl-N-ethylamine (5.27 mL, 30.3 mmol). The mixture was stirred for 15 min at rt. N,N-Diethylamine (3.12 mL; 30.2 mmol) was added, and the mixture was stirred at rt for 4 h. The mixture was poured into ice-water (300 mL), and a precipitate formed. The solid was separated via filtration, washed with water, and air-dried. The residue was purified via flash column chromatography (eluent gradient: 1 to 5% MeOH in CH₂Cl₂) to yield 7.93 g (96.8%) of 5-methoxy-9-oxo-9H-xanthene-3-carboxylic acid diethylamide 4c.

### 5-Methoxy-9-piperidin-4-ylidene-9H-xanthene-3-carboxylic acid diethylamide, 5c

Using an adaptation of the method described in Procedure 7, substituting 5-methoxy-9-oxo-9H-xanthene-3-carboxylic acid diethylamide 4c for 9-oxo-9H-xanthene-3-carboxylic acid diethylamide 6a, the title compound 5-methoxy-9-piperidin-4-ylidene-9H-xanthene-3-carboxylic acid diethylamide 5c was prepared as a TFA salt.

### 5-Methoxy-9-piperidin-4-yl-9H-xanthene-3-carboxylic acid diethylamide, 6c

Using an adaptation of the method described in Procedure 8, substituting 5-methoxy-9-piperidin-4-ylidene-9H-xanthene-3-carboxylic acid diethylamide 5c for 9-piperidin-4-ylidene-9H-xanthene-3-carboxylic acid diethylamide 7a, the title compound 5-methoxy-9-piperidin-4-yl-9H-xanthene-3-carboxylic acid diethylamide 6c was prepared as a TFA salt. MS m/z (MH⁺) 395.2.

### Procedure 15

### 5-Hydroxy-9-piperidin-4-yl-9H-xanthene-3-carboxylic acid diethylamide, 7c

To a solution of the TFA salt of 5-methoxy-9-piperidin-4-yl-9H-xanthene-3-carboxylic acid diethylamide, 6c (15 mg; 0.015 mmol) in CH₂Cl₂ (3 mL) at −78° C. was added a 1.0M BBr₃ in CH₂Cl₂ solution (0.03 mL; 0.03 mmol). The solution was allowed to warm to rt, MeOH (3 mL) was added, and the mixture was evaporated. Reverse phase chromatography (eluent: acetonitrile:water containing 0.1% TFA) yielded 7.2 mg (97.3%) of title compound 5-hydroxy-9-piperidin-4-yl-9H-xanthene-3-carboxylic acid diethylamide 7c as a TFA salt. MS m/z (MH⁺) 381.0.

### Example D

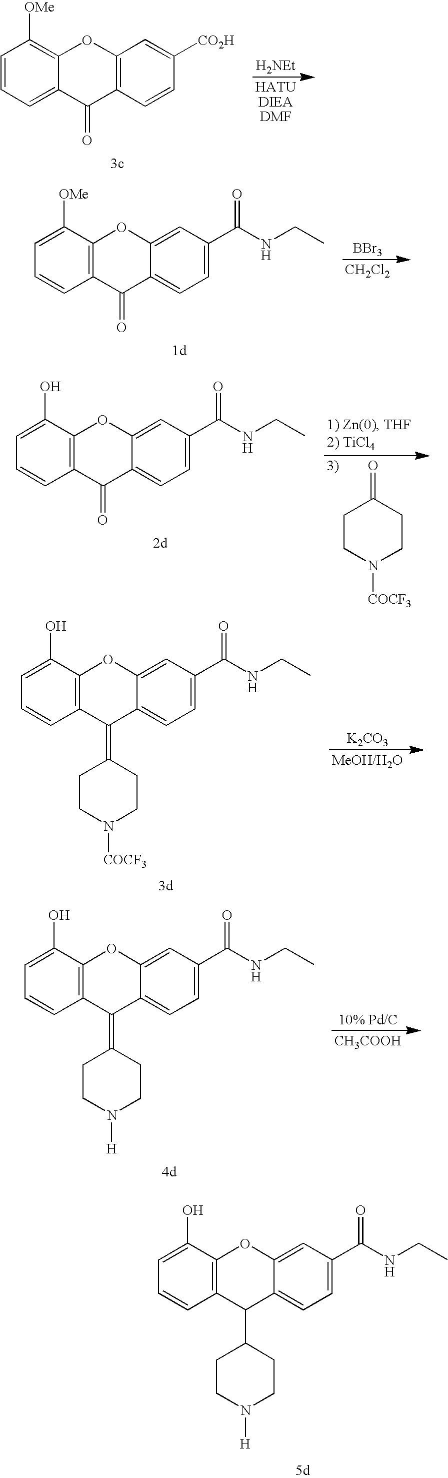

### Procedure 16

### 5-Methoxy-9-oxo-9H-xanthene-3-carboxylic acid ethylamide, 1d

To a suspension of 5-methoxy-9-oxo-9H-xanthene-3-carboxylic acid 3c (0.43 g, 1.59 mmol) and HATU (0.634 g; 1.67 mmol) in DMF (10 mL) was added N,N-diisopropyl-N-ethylamine (1.66 mL, 9.54 mmol). The mixture was stirred for 30 min at rt. Ethylamine hydrochloride (0.136 g; 1.67 mmol) was added, and the mixture was stirred at rt for 16 h. The mixture was poured into ice-water, and a precipitate formed. The solid was separated via filtration, washed with water, and air-dried, yielding 0.355 g (75%) of 5-methoxy-9-oxo-9H-xanthene-3-carboxylic acid ethylamide 1d. The product was used in the next reaction without further purification.

### 5-Hydroxy-9-oxo-9H-xanthene-3-carboxylic acid ethylamide, 2d

Using an adaptation of the method described in Procedure 15, substituting 5-methoxy-9-oxo-9H-xanthene-3-carboxylic acid ethylamide 1d for 5-methoxy-9-piperidin-4-yl-9H-xanthene-3-carboxylic acid diethylamide 6c, the title compound 5-hydroxy-9-oxo-9H-xanthene-3-carboxylic acid ethylamide 2d was prepared as a TFA salt.

### 5-Hydroxy-9-[1-(2,2,2-trifluoro-acetyl)-piperidin-4-ylidene]-9H-xanthene-3-carboxylic acid ethylamide, 3d

Using an adaptation of the method described in Procedure 7, substituting Compound 2d for Compound 6a and substituting 1-(2,2,2-trifluoroacetyl)-piperidin-4-one for 3-oxo-8-aza-bicyclo[3.2.1]-octane-8-carboxylic acid tert-butyl ester, the title compound 5-hydroxy-9-[1-(2,2,2-trifluoro-acetyl)-piperidin-4-ylidene]-9H-xanthene-3-carboxylic acid ethylamide 3d was prepared.

### Procedure 17

### 5-Hydroxy-9-piperidin-4-ylidene-9H-xanthene-3-carboxylic acid ethylamide, 4d

To a solution of Compound 5-hydroxy-9-[1-(2,2,2-trifluoro-acetyl)-piperidin-4-ylidene]-9H-xanthene-3-carboxylic acid ethylamide 3d (1.5 g, 3.36 mmol) in CH₃OH (30 mL) and H₂O (6 mL) was added K₂CO₃ (1.16 g, 8.39 mmol). The mixture was stirred for 5 h at rt and evaporated. The residue was purified by reverse phase HPLC to yield 0.6 g (38% from 1d) of 5-hydroxy-9-piperidin-4-ylidene-9H-xanthene-3-carboxylic acid ethylamide 4d as the TFA salt.

### Procedure 18

### 5-Hydroxy-9-piperidin-4-yl-9H-xanthene-3-carboxylic acid ethylamide, 5d

To a solution of 5-hydroxy-9-piperidin-4-ylidene-9H-xanthene-3-carboxylic acid ethylamide 4d (20.95 mg, 0.045 mmol) in acetic acid (3 mL) was added 10% palladium on carbon (15 mg), and the mixture was stirred under a hydrogen atmosphere for 16 h at rt. More catalyst (20 mg) was added, and the mixture was heated for at 35° C. for 5 h. The catalyst was removed via filtration, the solvent was evaporated, and the residue was purified via reverse phase HPLC (eluent: CH₃CN in H₂O containing 0.1% TFA) to yield 5.2 mg (25%) of 5-hydroxy-9-piperidin-4-yl-9H-xanthene-3-carboxylic acid ethylamide 5d as a TFA salt. MS m/z (MH⁺) 353.0.

### Example E

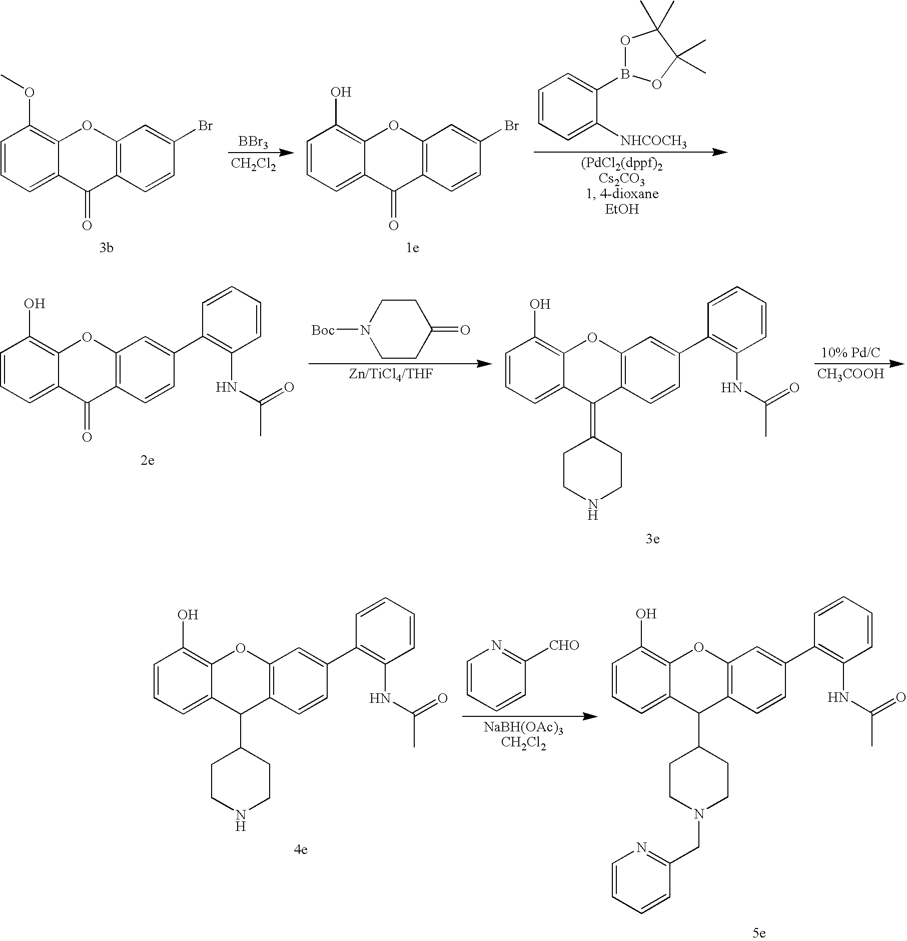

### 3-Bromo-5-hydroxy-xanthen-9-one, 1e

Using an adaptation of the method described in Procedure 15, substituting 3-bromo-5-methoxy-xanthen-9-one for 5-methoxy-9-piperidin-4-yl-9H-xanthene-3-carboxylic acid diethylamide 6c, the title compound 3-bromo-5-hydroxy-xanthen-9-one, 1e was obtained.

### Procedure 19

### N-[2-(5-Hydroxy-9-oxo-9H-xanthen-3-yl)-phenyl]-acetamide, 2e

A solution of 3-bromo-5-hydroxy-xanthen-9-one 1e (3.18 g, 10.9 mmol), N-[2-(4,4,5,5-tetramethyl-[1,3,2]dioxaborolan-2-yl)-phenyl]-acetamide (3.0 g, 11.5 mmol), PdCl₂(dppf)₂ (0.4 g, 0.55 mmol) and cesium carbonate (7.1 g, 21.8 mmol) in a mixture of dioxane (60 mL) and ethanol (20 mL) was heated to reflux for 3 hr. The mixture was allowed to cool to rt, filtered, and evaporated. The residue was diluted with water, and the precipitate was collected via filtration. After drying in a dessicator, 3.57 g (94.7%) of N-[2-(5-hydroxy-9-oxo-9H-xanthen-3-yl)-phenyl]-acetamide 2e was obtained and used as such for the next reaction.

### N-[2-(5-Hydroxy-9-piperidin-4-ylidene-9H-xanthen-3-yl)-phenyl]-acetamide, 3e

Using an adaptation of the method described in Procedure 7, substituting Compound 2e for Compound 6a, the title compound N-[2-(5-hydroxy-9-piperidin-4-ylidene-9H-xanthen-3-yl)-phenyl]-acetamide 3e was prepared.

### N-[2-(5-Hydroxy-9-piperidin-4-yl-9H-xanthen-3-yl)-phenyl]-acetamide, 4e

Using an adaptation of the method described in Procedure 18, substituting N-[2-(5-hydroxy-9-piperidin-4-ylidene-9H-xanthen-3-yl)-phenyl]-acetamide 3e for Compound 4d, the title compound N-[2-(5-hydroxy-9-piperidin-4-yl-9H-xanthen-3-yl)-phenyl]-acetamide 4e was prepared as a TFA salt. MS m/z (MH⁺) 414.9. ¹H NMR (CD₃OD) δ 7.5-7.3 (m, 6H); 7.15 (d, 1H); 7.0 (d, 2H); 6.8 (dd, 2H); 4.0 (d, 1H); 2.9 (m, 2H); 2-1.8 (m, s, 6H); 1.4 (m, 2H).

### N-{2-[5-Hydroxy-9-(1-pyridin-2-ylmethyl-piperidin-4-yl)-9H-xanthen-3-yl]-phenyl}-acetamide, 5e

Using an adaptation of the method described in Procedure 9, substituting the TFA salt of N-[2-(5-hydroxy-9-piperidin-4-yl-9H-xanthen-3-yl)-phenyl]-acetamide 4e for 9-piperidin-4-yl-9H-xanthene-3-carboxylic acid diethylamide, 8a, 2-pyridyl carboxaldehyde for 3-furaldehyde, sodium triacetoxyborohydride for tetrabutylammonium triacetoxyborohydride, and without adding N,N-diisopropyl-N-ethylamine, the title compound N-{2-[5-hydroxy-9-(1-pyridin-2-ylmethyl-piperidin-4-yl)-9H-xanthen-3-yl]-phenyl}-acetamide, 5e was prepared as a TFA salt. MS m/z (MH⁺) 506.2.

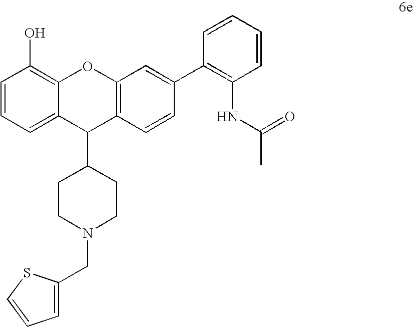

### N-{2-[5-Hydroxy-9-(1-thiophen-2-ylmethyl-piperidin-4-yl)-9H-xanthen-3-yl]-phenyl}-acetamide, 6e

Using an adaptation of the method described in Procedure 9, substituting the TFA salt of N-[2-(5-hydroxy-9-piperidin-4-yl-9H-xanthen-3-yl)-phenyl]-acetamide 4e for 9-piperidin-4-yl-9H-xanthene-3-carboxylic acid diethylamide, 8a, 2-thiophene carboxaldehyde for 3-furaldehyde, sodium triacetoxyborohydride for tetrabutylammonium triacetoxyborohydride, and without adding N,N-diisopropyl-N-ethylamine, the title compound N-{2-[5-hydroxy-9-(1-thiophen-2-ylmethyl-piperidin-4-yl)-9H-xanthen-3-yl]-phenyl}-acetamide, 6e was prepared as a TFA salt. MS m/z (MH⁺) 511.2.

### Example F

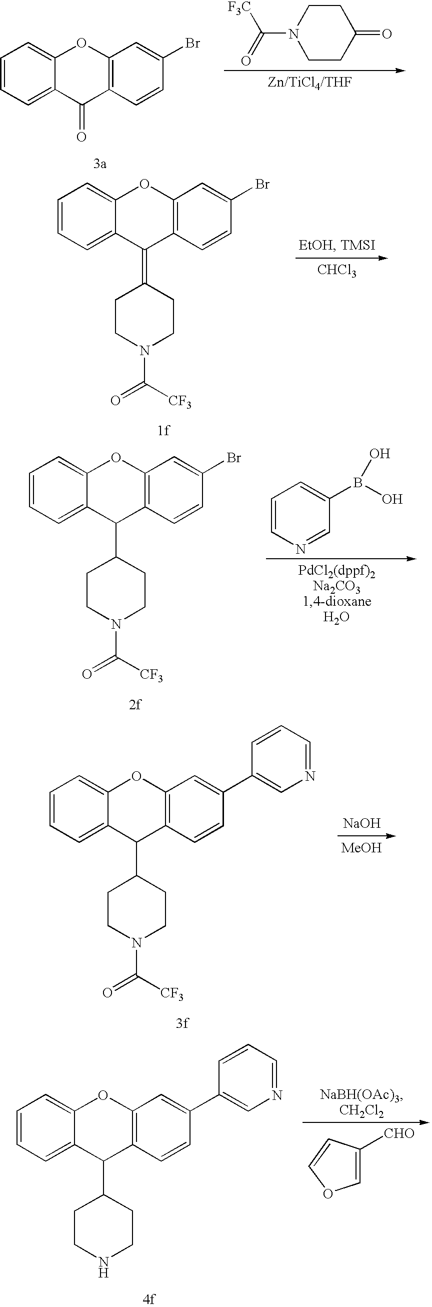

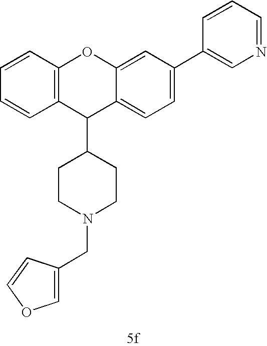

### 1-[4-(3-Bromo-xanthen-9-ylidene)-piperidin-1-yl]-2,2,2-trifluoro-ethanone, 1f

Using an adaptation of the method described in Procedure 7, substituting Compound 3a for Compound 6a and substituting 1-(2,2,2-trifluoroacetyl)piperidin-4-one for 3-oxo-8-aza-bicyclo[3.2.1]-octane-8-carboxylic acid tert-butyl ester, the title compound 1-[4-(3-bromo-xanthen-9-ylidene)-piperidin-1-yl]-2,2,2-trifluoro-ethanone 1f was prepared. MS m/z (MH⁺) 440.7.

### 1-[4-(3-Bromo-9H-xanthen-9-yl)-piperidin-1-yl]-2,2,2-trifluoro-ethanone, 2f

Using an adaptation of the method described in Procedure 8, substituting 1-[4-(3-bromo-xanthen-9-ylidene)-piperidin-1-yl]-2,2,2-trifluoro-ethanone 1f for 9-piperidin-4-ylidene-9H-xanthene-3-carboxylic acid diethylamide 7a, the title compound 1-[4-(3-bromo-9H-xanthen-9-yl)-piperidin-1-yl]-2,2,2-trifluoro-ethanone 2f was prepared. MS m/z (MH⁺) 439.6/440.7.

### 2,2,2-Trifluoro-1-[4-(3-pyridin-3-yl-9H-xanthen-9-yl)-piperidin-1-yl]-ethanone, 3f

Using an adaptation of the method described in Procedure 19, substituting 1-[4-(3-bromo-9H-xanthen-9-yl)-piperidin-1-yl]-2,2,2-trifluoro-ethanone 2f for 3-bromo-5-hydroxy-xanthen-9-one 1e, 3-pyridyl boronic acid for N-[2-(4,4,5,5-tetramethyl-[1,3,2]dioxaborolan-2-yl)-phenyl]-acetamide and sodium carbonate in water for cesium carbonate, the title compound 2,2,2-trifluoro-1-[4-(3-pyridin-3-yl-9H-xanthen-9-yl)-piperidin-1-yl]-ethanone 3f was prepared.

### 3-(9-Piperidin-4-yl-9H-xanthen-3-yl)-pyridine, 4f

Using an adaptation of the method described in Procedure 17, substituting 2,2,2-trifluoro-1-[4-(3-pyridin-3-yl-9H-xanthen-9-yl)-piperidin-1-yl]-ethanone 3f for 5-hydroxy-9-[1-(2,2,2-trifluoro-acetyl)-piperidin-4-ylidene]-9H-xanthene-3-carboxylic acid ethylamide 3d, and sodium hydroxide for potassium carbonate, the title compound 3-(9-piperidin-4-yl-9H-xanthen-3-yl)-pyridine 4f was obtained. MS m/z (MH⁺) 343.1.

### 3-[9-(1-Furan-3-ylmethyl-piperidin-4-yl)-9H-xanthen-3-yl]-pyridine, 5f

Using an adaptation of the method described in Procedure 9, substituting 3-(9-piperidin-4-yl-9H-xanthen-3-yl)-pyridine 4f for 9-piperidin-4-yl-9H-xanthene-3-carboxylic acid diethylamide 8a, the title compound 3-[9-(1-furan-3-ylmethyl-piperidin-4-yl)-9H-xanthen-3-yl]-pyridine 5f was obtained. MS m/z (MH⁺) 422.9.

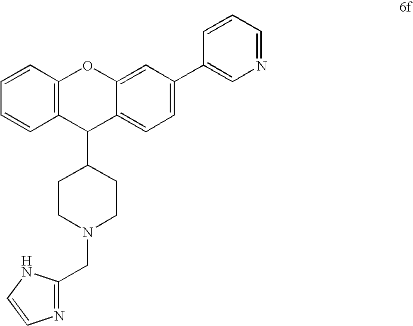

### 3-{9-[1-(1H-Imidazol-2-ylmethyl)-piperidin-4-yl]-9H-xanthen-3-yl}-pyridine, 6f

Using an adaptation of the method described in Procedure 9, substituting 3-(9-piperidin-4-yl-9H-xanthen-3-yl)-pyridine 4f for 9-piperidin-4-yl-9H-xanthene-3-carboxylic acid diethylamide 8a, and 1H-imidazole-2-carboxaldehyde for 3-furaldehyde, the title compound 3-{9-[1-(1H-imidazol-2-ylmethyl)-piperidin-4-yl]-9H-xanthen-3-yl}-pyridine, 6f was obtained. MS m/z (MH⁺) 422.9.

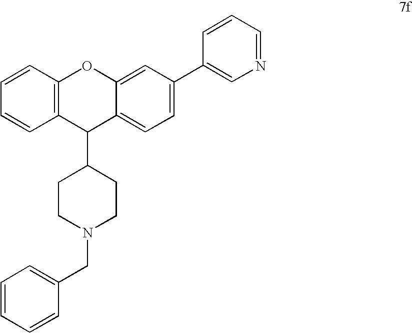

### 3-[9-(1-Benzyl-piperidin-4-yl)-9H-xanthen-3-yl]-pyridine, 7f

Using an adaptation of the method described in Procedure 9, substituting 3-(9-piperidin-4-yl-9H-xanthen-3-yl)-pyridine, 4f for 9-piperidin-4-yl-9H-xanthene-3-carboxylic acid diethylamide, 8a, and benzaldehyde for 3-furaldehyde, the title compound 3-[9-(1-benzyl-piperidin-4-yl)-9H-xanthen-3-yl]-pyridine, 7f was obtained. MS m/z (MH⁺) 432.9.

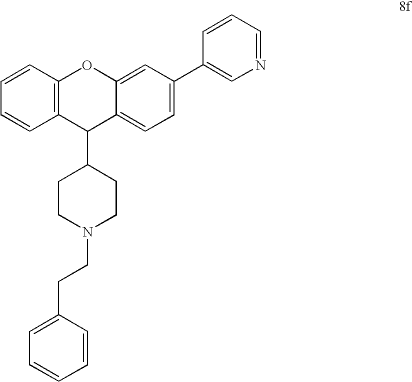

### 3-[9-(1-Phenethyl-piperidin-4-yl)-9H-xanthen-3-yl]-pyridine, 8f

Using an adaptation of the method described in Procedure 9, substituting 3-(9-piperidin-4-yl-9H-xanthen-3-yl)-pyridine, 4f for 9-piperidin-4-yl-9H-xanthene-3-carboxylic acid diethylamide, 8a, and phenylacetaldehyde for 3-furaldehyde, the title compound 3-[9-(1-phenethyl-piperidin-4-yl)-9H-xanthen-3-yl]-pyridine, 8f was obtained. MS m/z (MH⁺) 446.9.

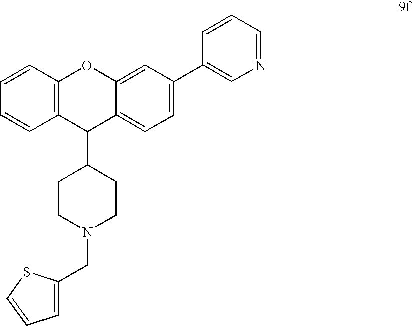

### 3-[9-(1-Thiophen-2-ylmethyl-piperidin-4-yl)-9H-xanthen-3-yl]-pyridine, 9f

Using an adaptation of the method described in Procedure 9, substituting 3-(9-piperidin-4-yl-9H-xanthen-3-yl)-pyridine, 4f for 9-piperidin-4-yl-9H-xanthene-3-carboxylic acid diethylamide, 8a, and 2-thiophene carboxaldehyde for 3-furaldehyde, the title compound 3-[9-(1-thiophen-2-ylmethyl-piperidin-4-yl)-9H-xanthen-3-yl]-pyridine, 9f was obtained as a TFA salt. The salt was dissolved in ethyl acetate, and the solution was washed with 1 N NaOH. The organic layer was separated, dried over potassium carbonate, filtered, and evaporated. The residue was dissolved in diethyl ether, and treated with a 1 N HCl in ether solution to. After evaporation, the title compound 3-[9-(1-thiophen-2-ylmethyl-piperidin-4-yl)-9H-xanthen-3-yl]-pyridine, 9f was isolated as the HCl salt. MS m/z (MH⁺) 438.9.

### Example G

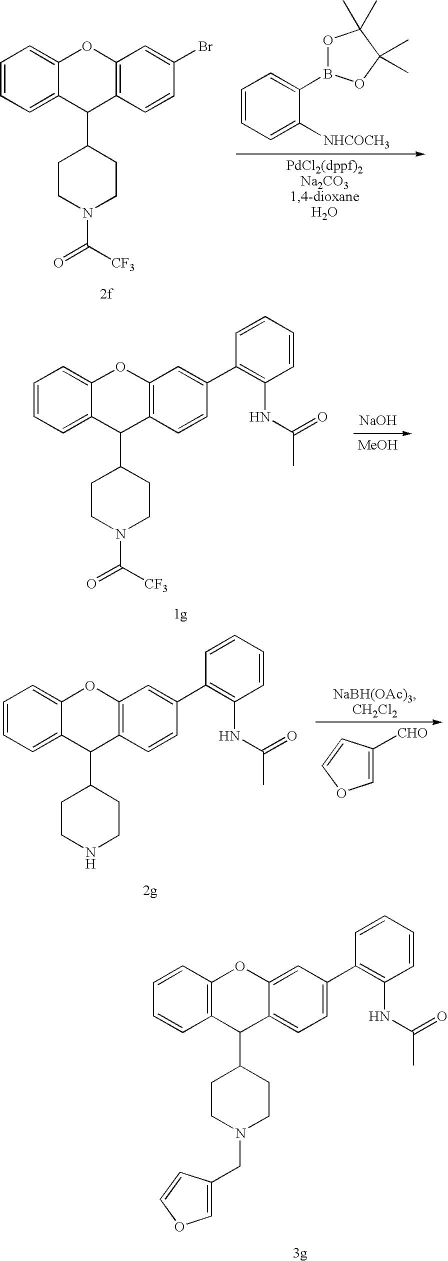

### N-(2-{9-[1-(2,2,2-Trifluoroacetyl)-piperidin-4-yl]-9H-xanthen-3-yl}-phenyl)acetamide, 1g

Using an adaptation of the method described in Procedure 19, substituting 1-[4-(3-bromo-9H-xanthen-9-yl)-piperidin-1-yl]-2,2,2-trifluoro-ethanone, 2f for 3-bromo-5-hydroxy-xanthen-9-one 1e, and sodium carbonate in water for cesium carbonate, the title compound N-(2-{9-[1-(2,2,2-trifluoroacetyl)-piperidin-4-yl]-9H-xanthen-3-yl}-phenyl)-acetamide, 1g was prepared.

### N-[2-(9-Piperidin-4-yl-9H-xanthen-3-yl)-phenyl]-acetamide, 2g

Using an adaptation of the method described in Procedure 17, substituting N-(2-{9-[1-(2,2,2-trifluoroacetyl)-piperidin-4-yl]-9H-xanthen-3-yl}-phenyl)acetamide, 1g for 5-hydroxy-9-[1-(2,2,2-trifluoro-acetyl)-piperidin-4-ylidene]-9H-xanthene-3-carboxylic acid ethylamide 3d, and sodium hydroxide for potassium carbonate, the title compound N-[2-(9-piperidin-4-yl-9H-xanthen-3-yl)-phenyl]-acetamide, 2g was obtained. MS m/z (MH⁺) 399.2.

### N-{2-[9-(1-Furan-3-ylmethyl-piperidin-4-yl)-9H-xanthen-3-yl]-phenyl}-acetamide, 3g

Using an adaptation of the method described in Procedure 9, substituting N-[2-(9-piperidin-4-yl-9H-xanthen-3-yl)-phenyl]-acetamide, 2g for 9-piperidin-4-yl-9H-xanthene-3-carboxylic acid diethylamide 8a, the title compound N-{2-[9-(1-furan-3-ylmethyl-piperidin-4-yl)-9H-xanthen-3-yl]-phenyl}-acetamide, 3g was obtained. MS m/z (MH⁺) 479.0.

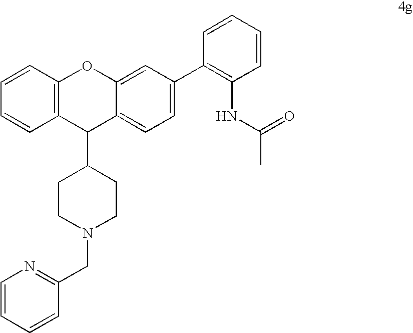

### N-{2-[9-(1-Pyridin-2-ylmethyl-piperidin-4-yl)-9H-xanthen-3-yl]-phenyl}-acetamide, 4g

Using an adaptation of the method described in Procedure 9, substituting N-[2-(9-piperidin-4-yl-9H-xanthen-3-yl)-phenyl]-acetamide, 2g for 9-piperidin-4-yl-9H-xanthene-3-carboxylic acid diethylamide 8a, and 2-pyridylcarboxaldehyde for 3-furaldehyde, the title compound N-{2-[9-(1-pyridin-2-ylmethyl-piperidin-4-yl)-9H-xanthen-3-yl]-phenyl}-acetamide, 4g was obtained. MS m/z (MH⁺) 489.9.

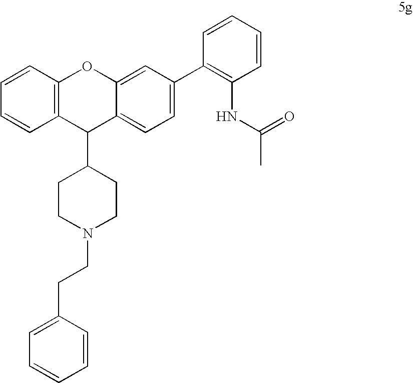

### N-{2-[9-(1-Phenethyl-piperidin-4-yl)-9H-xanthen-3-yl]-phenyl}-acetamide, 5g

Using an adaptation of the method described in Procedure 9, substituting N-[2-(9-Piperidin-4-yl-9H-xanthen-3-yl)-phenyl]-acetamide, 2g for 9-piperidin-4-yl-9H-xanthene-3-carboxylic acid diethylamide 8a, and phenylacetaldehyde for 3-furaldehyde, the title compound N-{2-[9-(1-phenethyl-piperidin-4-yl)-9H-xanthen-3-yl]-phenyl}-acetamide, 5g was obtained. MS m/z (MH⁺) 503.0.

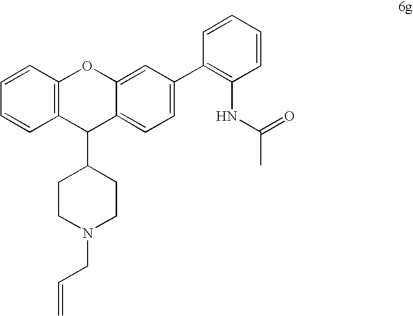

### Procedure 20

### N-{2-[9-(1-Allyl-piperidin-4-yl)-9H-xanthen-3-yl]-phenyl}-acetamide, 6g

To a solution of N-[2-(9-piperidin-4-yl-9H-xanthen-3-yl)-phenyl]-acetamide, 2g (0.034 g, 0.085 mmol) in CH₃CN (3 mL) were added potassium carbonate (0.035 g, 0.255 mmol) and allyl bromide (7 μL, 0.085 mmol). The mixture was allowed to stir for 3 h at rt, and the solid was removed via filtration. The filtrate was evaporated, and the residue was triturated with water. The solid was separated via filtration, washed with water, and air-dried, yielding 5.3 mg (14%) of title compound N-{2-[9-(1-allyl-piperidin-4-yl)-9H-xanthen-3-yl]-phenyl}-acetamide, 6g. MS m/z (MH⁺) 439.2.

### Example H

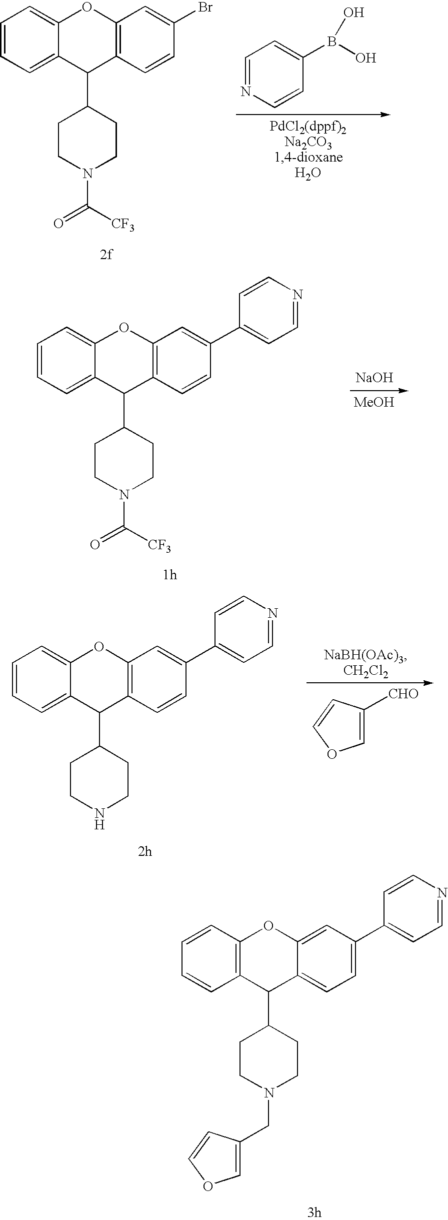

### 2,2,2-Trifluoro-1-[4-(3-pyridin-4-yl-9H-xanthen-9-yl)-piperidin-1-yl]-ethanone, 1 h

Using an adaptation of the method described in Procedure 19, substituting 1-[4-(3-bromo-9H-xanthen-9-yl)-piperidin-1-yl]-2,2,2-trifluoro-ethanone 2f for 3-bromo-5-hydroxy-xanthen-9-one 1e, 4-pyridyl boronic acid for N-[2-(4,4,5,5-tetramethyl-[1,3,2]dioxaborolan-2-yl)-phenyl]-acetamide and sodium carbonate in water for cesium carbonate, the title compound 2,2,2-Trifluoro-1-[4-(3-pyridin-4-yl-9H-xanthen-9-yl)-piperidin-1-yl]-ethanone, 1 h was prepared.

### 4-(9-Piperidin-4-yl-9H-xanthen-3-yl)-pyridine, 2h

Using an adaptation of the method described in Procedure 17, substituting 2,2,2-trifluoro-1-[4-(3-pyridin-3-yl-9H-xanthen-9-yl)-piperidin-1-yl]-ethanone 3f for 5-hydroxy-9-[1-(2,2,2-trifluoro-acetyl)-piperidin-4-ylidene]-9H-xanthene-3-carboxylic acid ethylamide 3d, and sodium hydroxide for potassium carbonate, the title compound 4-(9-piperidin-4-yl-9H-xanthen-3-yl)-pyridine, 2h was obtained. MS m/z (MH⁺) 343.1.

### 4-[9-(1-Furan-3-ylmethyl-piperidin-4-yl)-9H-xanthen-3-yl]-pyridine, 3h

Using an adaptation of the method described in Procedure 9, substituting 4-(9-piperidin-4-yl-9H-xanthen-3-yl)-pyridine, 2h for 9-piperidin-4-yl-9H-xanthene-3-carboxylic acid diethylamide 8a, the title compound 4-[9-(1-furan-3-ylmethyl-piperidin-4-yl)-9H-xanthen-3-yl]-pyridine, 3h was obtained. MS m/z (MH⁺) 422.9.

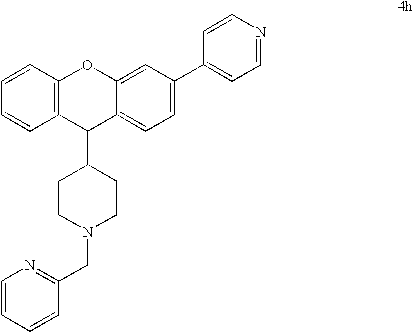

### 4-[9-(1-Pyridin-2-ylmethyl-piperidin-4-yl)-9H-xanthen-3-yl]-pyridine, 4h

Using an adaptation of the method described in Procedure 9, substituting 4-[9-(1-furan-3-ylmethyl-piperidin-4-yl)-9H-xanthen-3-yl]-pyridine, 3h for 9-piperidin-4-yl-9H-xanthene-3-carboxylic acid diethylamide 8a, and 2-pyridyl carboxaldehyde for 3-furaldehyde, the title compound 4-[9-(1-pyridin-2-ylmethyl-piperidin-4-yl)-9H-xanthen-3-yl]-pyridine, 4h was obtained. MS m/z (MH⁺) 434.0.

### Example I

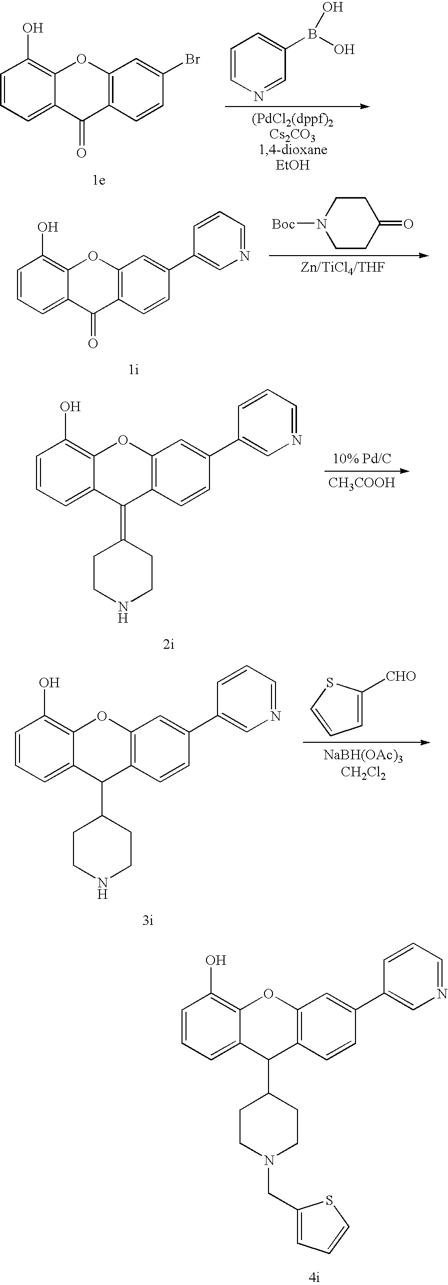

### 5-Hydroxy-3-pyridin-3-yl-xanthen-9-one, 1i

Using an adaptation of the method described in Procedure 19, substituting 3-pyridyl boronic acid for N-[2-(4,4,5,5-tetramethyl-[1,3,2]dioxaborolan-2-yl)phenyl]-acetamide, the title compound 5-hydroxy-3-pyridin-3-yl-xanthen-9-one, 1i was prepared.

### 9-Piperidin-4-ylidene-6-pyridin-3-yl-9H-xanthen-4-ol, 2i

Using an adaptation of the method described in Procedure 7, substituting 5-hydroxy-3-pyridin-3-yl-xanthen-9-one, 11 for 9-oxo-9H-xanthene-3-carboxylic acid diethylamide, 6a, the title compound 9-piperidin-4-ylidene-6-pyridin-3-yl-9H-xanthen-4-ol, 2i was prepared.

### 9-Piperidin-4-yl-6-pyridin-3-yl-9H-xanthen-4-ol, 3i

Using an adaptation of the method described in Procedure 18, substituting 9-piperidin-4-ylidene-6-pyridin-3-yl-9H-xanthen-4-ol, 2i for 5-hydroxy-9-piperidin-4-ylidene-9H-xanthene-3-carboxylic acid ethylamide 4d, the title compound 9-piperidin-4-yl-6-pyridin-3-yl-9H-xanthen-4-ol, 3i was prepared as a TFA salt. MS m/z (MH⁺) 359.0

### 6-Pyridin-3-yl-9-(1-thiophen-2-ylmethyl-piperidin-4-yl)-9H-xanthen-4-ol, 4i

Using an adaptation of the method described in Procedure 9, substituting the TFA salt of 9-piperidin-4-yl-6-pyridin-3-yl-9H-xanthen-4-ol, 3i for 9-piperidin-4-yl-9H-xanthene-3-carboxylic acid diethylamide, 8a, 2-thiophene carboxaldehyde for 3-furaldehyde, sodium triacetoxyborohydride for tetrabutylammonium triacetoxyborohydride, and without adding N,N-diisopropyl-N-ethylamine, the title compound 6-pyridin-3-yl-9-(1-thiophen-2-ylmethyl-piperidin-4-yl)-9H-xanthen-4-ol, 4i was prepared as a TFA salt. MS m/z (MH⁺) 455.1.

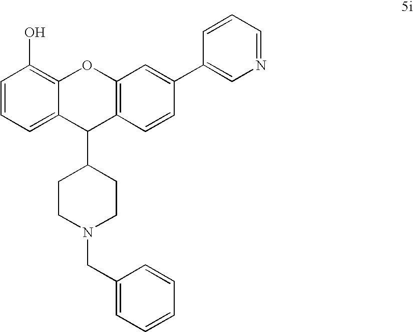

### 9-(1-Benzyl-piperidin-4-yl)-6-pyridin-3-yl-9H-xanthen-4-ol, 5i

Using an adaptation of the method described in Procedure 9, substituting the TFA salt of 9-piperidin-4-yl-6-pyridin-3-yl-9H-xanthen-4-ol, 3i for 9-piperidin-4-yl-9H-xanthene-3-carboxylic acid diethylamide, 8a, phenyl acetaldehyde for 3-furaldehyde, sodium triacetoxyborohydride for tetrabutylammonium triacetoxyborohydride, and without adding N,N-diisopropyl-N-ethylamine, the title compound 6-pyridin-3-yl-9-(1-thiophen-2-ylmethyl-piperidin-4-yl)-9H-xanthen-4-ol, 4i was prepared as a TFA salt. MS m/z (MH⁺) 449.1.

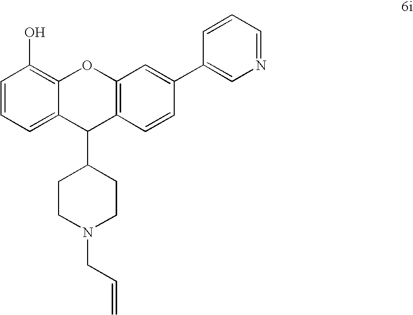

### 9-(1-Allyl-piperidin-4-yl)-6-pyridin-3-yl-9H-xanthen-4-ol, 6i

Using an adaptation of the method described in Procedure 20, substituting the TFA salt of 9-piperidin-4-yl-6-pyridin-3-yl-9H-xanthen-4-ol, 31 for N-[2-(9-piperidin-4-yl-9H-xanthen-3-yl)-phenyl]-acetamide, 2g, the title compound 9-(1-allyl-piperidin-4-yl)-6-pyridin-3-yl-9H-xanthen-4-ol, 61 was obtained as a TFA salt after reverse phase HPLC purification (eluent: acetonitrile in water containing 0.1% TFA). MS m/z (MH⁺) 399.1.

### Example J

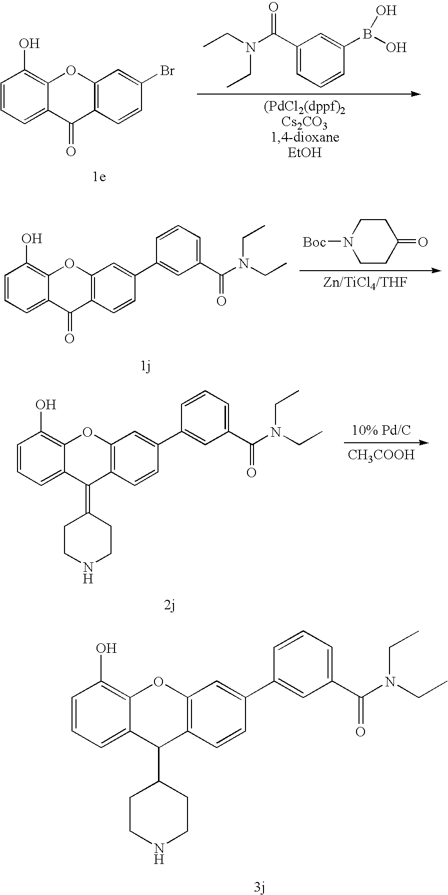

### N,N-Diethyl-3-(5-hydroxy-9-oxo-9H-xanthen-3-yl)-benzamide, 1j

Using an adaptation of the method described in Procedure 19, substituting 3-(diethylaminocarbonyl)phenyl boronic acid for N-[2-(4,4,5,5-tetramethyl-[1,3,2]dioxaborolan-2-yl)-phenyl]-acetamide, the title compound N,N-diethyl-3-(5-hydroxy-9-oxo-9H-xanthen-3-yl)-benzamide, 1j was prepared.

### N,N-Diethyl-3-(5-hydroxy-9-piperidin-4-ylidene-9H-xanthen-3-yl)benzamide, 2j

Using an adaptation of the method described in Procedure 7, N,N-diethyl-3-(5-hydroxy-9-oxo-9H-xanthen-3-yl)-benzamide, 1j for 9-oxo-9H-xanthene-3-carboxylic acid diethylamide, 6a, the title compound N,N-diethyl-3-(5-hydroxy-9-piperidin-4-ylidene-9H-xanthen-3-yl)-benzamide, 2j was prepared.

### N,N-Diethyl-3-(5-hydroxy-9-piperidin-4-yl-9H-xanthen-3-yl)-benzamide, 3j

Using an adaptation of the method described in Procedure 18, substituting N,N-diethyl-3-(5-hydroxy-9-piperidin-4-ylidene-9H-xanthen-3-yl)-benzamide, 2j for 5-hydroxy-9-piperidin-4-ylidene-9H-xanthene-3-carboxylic acid ethylamide 4d, the title compound N,N-diethyl-3-(5-hydroxy-9-piperidin-4-yl-9H-xanthen-3-yl)benzamide, 3j was prepared as a TFA salt. MS m/z (MH⁺) 456.9. ¹H NMR (CD₃OD) [symbol] 7.8 ( [symbol] , 1H); 7.7-7.4 (m, 6H); 7.0 (m, 1H); 6.85 (m, 1H); 6.8 (m, 1H); 4.05 (d, 1H); 3.6 (q, 2H); 3.3 (m, 3H); 2.85 (q, 2H); 2.0-1.7 (m, dd, 4H); 1.45 (m, 2H); 1.3 (t, 3H); 1.15 (t, 3H).

### Example K

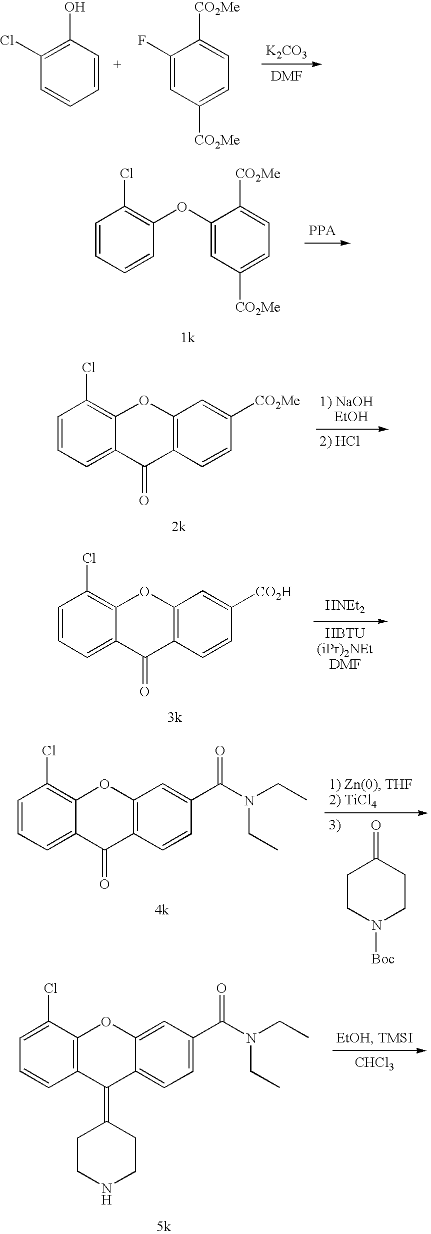

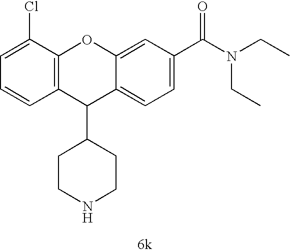

### 2-(2-Chlorophenoxy)-terephthalic acid dimethyl ester, 1k

Using an adaptation of the method described in Procedure 1, substituting 2-chlorophenol for phenol and potassium carbonate for sodium hydride, the title compound 2-(2-chlorophenoxy)-terephthalic acid dimethyl ester, 1 k was prepared.

### 5-Chloro-9-oxo-9H-xanthene-3-carboxylic acid methyl ester, 2k

Using an adaptation of the method described in Procedure 12, substituting 2-(2-chlorophenoxy)-terephthalic acid dimethyl ester, 1 k for 2-(2-methoxyphenoxy)-terephthalic acid dimethyl ester, 1c, the title compound 5-chloro-9-oxo-9H-xanthene-3-carboxylic acid methyl ester, 2k was obtained.

### 5-Chloro-9-oxo-9H-xanthene-3-carboxylic acid, 3k

Using an adaptation of the method described in Procedure 5, substituting 5-chloro-9-oxo-9H-xanthene-3-carboxylic acid methyl ester, 2k for 9-oxo-9H-xanthene-3-carboxylic acid methyl ester, 4a, title compound 5-chloro-9-oxo-9H-xanthene-3-carboxylic acid, 3k was obtained.

### 5-Chloro-9-oxo-9H-xanthene-3-carboxylic acid diethylamide, 4k

Using an adaptation of the method described in Procedure 16, substituting 5-chloro-9-oxo-9H-xanthene-3-carboxylic acid, 3k for 5-methoxy-9-oxo-9H-xanthene-3-carboxylic acid, 3d, and N,N-diethylamine for ethylamine, the title compound 5-chloro-9-oxo-9H-xanthene-3-carboxylic acid diethylamide, 4k was obtained.

### 5-Chloro-9-piperidin-4-ylidene-9H-xanthene-3-carboxylic acid diethylamide, 5k

Using an adaptation of the method described in Procedure 7, substituting 5-chloro-9-oxo-9H-xanthene-3-carboxylic acid diethylamide, 4k for 9-oxo-9H-xanthene-3-carboxylic acid diethylamide, 6a, title compound 5-chloro-9-piperidin-4-ylidene-9H-xanthene-3-carboxylic acid diethylamide, 5k was obtained as a TFA salt after reverse phase HPLC purification (eluent gradient: CH₃CN in H₂O containing 0.1% TFA).

### 5-Chloro-9-piperidin-4-yl-9H-xanthene-3-carboxylic acid diethylamide, 6k

Using an adaptation of the method described in Procedure 9, substituting the TFA salt of 5-chloro-9-piperidin-4-ylidene-9H-xanthene-3-carboxylic acid diethylamide, 5k for 9-piperidin-4-yl-9H-xanthene-3-carboxylic acid diethylamide, 8a, the title compound 5-chloro-9-piperidin-4-yl-9H-xanthene-3-carboxylic acid diethylamide, 6k was obtained as a TFA salt after reverse phase HPLC purification (eluent gradient: CH₃CN in H₂O containing 0.1% TFA). MS m/z (MH⁺) 399.1.

### Example L

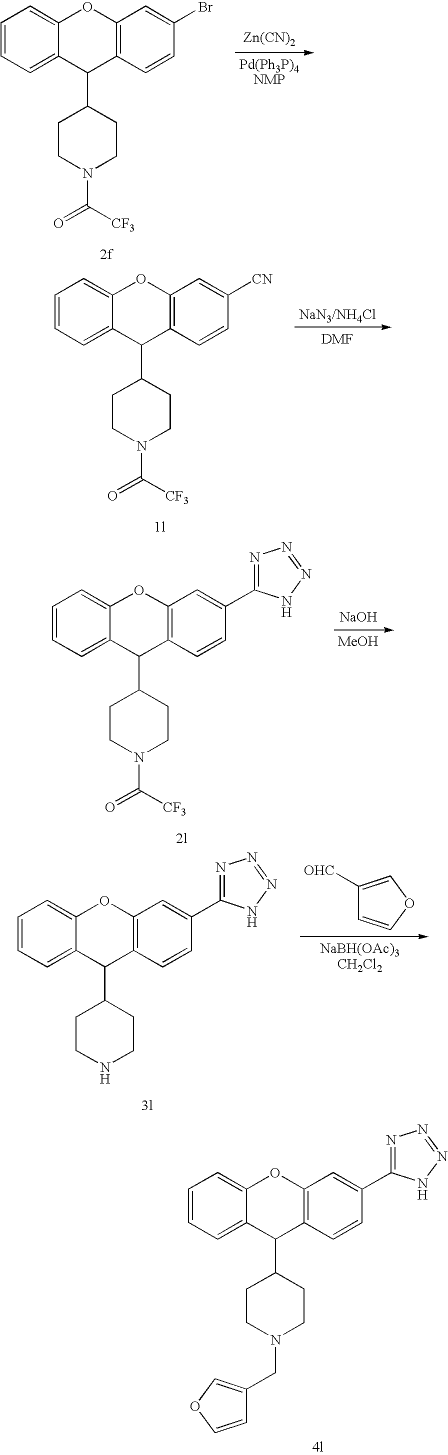

### Procedure 21

### 9-[1-(2,2,2-Trifluoroacetyl)-piperidin-4-yl]-9H-xanthene-3-carbonitrile, 1l

To a solution of 1-[4-(3-bromo-9H-xanthen-9-yl)-piperidin-1-yl]-2,2,2-trifluoro-ethanone, 2f (1.71 g, 3.85 mmol) in N-methylpyrrolidine (50 mL) was added zinc cyanide (0.27 g, 2.31 mmol), and the mixture was degassed for 15 min with Argon. Tetrakis(triphenylphosphine)palladium (0.2 g, 0.19 mmol) was added, and the mixture was heated for 5 h at 100° C., followed by stirring at rt overnight. The mixture was diluted with water, and extracted with ethyl acetate. The organic phase was washed with brine, dried over MgSO₄, filtered, and evaporated. The residue was purified via flash column chromatography (eluent gradient: 10% to 30% ethyl acetate in heptane) to yield 1.3 g (87.5%) of title compound 9-[1-(2,2,2-trifluoroacetyl)-piperidin-4-yl]-9H-xanthene-3-carbonitrile, 1l. MS m/z (MH⁺) 386.8.

### Procedure 22

### 2,2,2-Trifluoro-1-{4-[3-(1H-tetrazol-5-yl)-9H-xanthen-9-yl]-piperidin-1-yl}-ethanone, 2l

To a solution of 9-[1-(2,2,2-trifluoroacetyl)-piperidin-4-yl]-9H-xanthene-3-carbonitrile, 1l (0.5 g; 1.3 mmol) in DMF (10 mL) were added sodium azide (0.25 g, 3.9 mmol) and ammonium chloride (0.21 g; 3.9 mmol), and the mixture was heated at 120° C. for 3 h. The mixture was allowed to cool to rt, poured into water, and the solid was separated via filtration, yielding 0.42 g (75.3%) of title compound 2,2,2-trifluoro-1-{4-[3-(1H-tetrazol-5-yl)-9H-xanthen-9-yl]-piperidin-1-yl}-ethanone, 21. MS m/z (MH⁺) 430.

### Procedure 23

### 4-[3-(1H-Tetrazol-5-yl)-9H-xanthen-9-yl]-piperidine, 3l

To a solution of 2,2,2-trifluoro-1-{4-[3-(1H-tetrazol-5-yl)-9H-xanthen-9-yl]-piperidin-1-yl}-ethanone, 2l (0.42 g, 0.98 mmol) in methanol (5 mL) was added a 3N NaOH solution (0.75 mL). The mixture was heated to reflux for 1 h, and the solvent was evaporated. The residue was purified via reverse phase HPLC (eluent: acetonitrile in water containing 0.1% TFA) to yield 34.3 mg (8%) of title compound 4-[3-(1H-tetrazol-5-yl)-9H-xanthen-9-yl]-piperidine, 3l as a TFA salt. MS m/z (MH⁺) 333.9.

### 1-Furan-3-ylmethyl-4-[3-(1H-tetrazol-5-yl)-9H-xanthen-9-yl]-piperidine, 4l

Using an adaptation of the method described in Procedure 9, substituting the TFA salt of 4-[3-(1H-tetrazol-5-yl)-9H-xanthen-9-yl]-piperidine, 3l for 9-piperidin-4-yl-9H-xanthene-3-carboxylic acid diethylamide 8a, the title compound 1-furan-3-ylmethyl-4-[3-(1H-tetrazol-5-yl)-9H-xanthen-9-yl]-piperidine, 4l was obtained as a TFA salt after reverse phase HPLC purification (eluent: acetonitrile in water containing 0.1% TFA). MS m/z (MH⁺)-414.1.

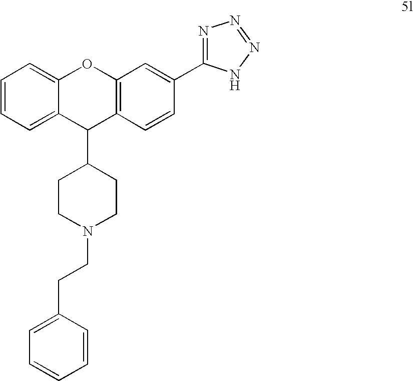

### 1-Phenethyl-4-[3-(1H-tetrazol-5-yl)-9H-xanthen-9-yl]-piperidine, 5l

Using an adaptation of the method described in Procedure 9, substituting the TFA salt of 4-[3-(1H-tetrazol-5-yl)-9H-xanthen-9-yl]-piperidine, 3l for 9-piperidin-4-yl-9H-xanthene-3-carboxylic acid diethylamide 8a, and phenyl acetaldehyde for 3-furyl carboxaldehyde, the title compound 1-phenethyl-4-[3-(1H-tetrazol-5-yl)-9H-xanthen-9-yl]-piperidine, 4l was obtained as a TFA salt after reverse phase HPLC purification (eluent: acetonitrile in water containing 0.1% TFA). MS m/z (MH⁺) 438.2.

### Example M

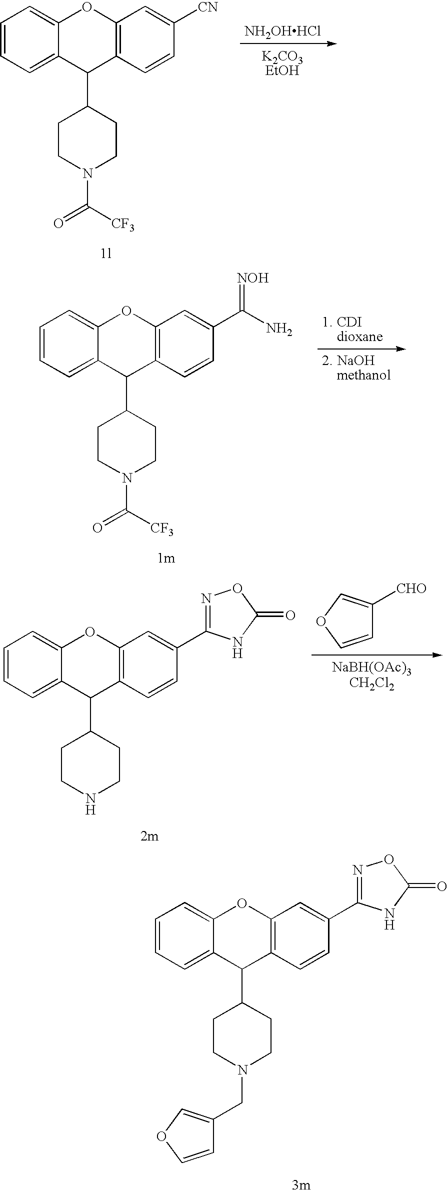

### Procedure 24

### N-Hydroxy-9-[1-(2,2,2-trifluoroacetyl)-piperidin-4-yl]-9H-xanthene-3-carboxamidine, 1m

To a solution of 9-[1-(2,2,2-trifluoroacetyl)-piperidin-4-yl]-9H-xanthene-3-carbonitrile, 1l (0.51 g; 1.3 mmol) in ethanol (10 mL) were added ammonium hydroxide hydrochloride (0.27 g; 3.9 mmol) and potassium carbonate (0.36 g; 2.6 mmol), and the mixture was heated to 90° C. for 16 h. The mixture was allowed to cool to rt, water (10 mL) was added, and the mixture was extracted with ethyl acetate. The organic phase was dried over MgSO₄, filtered, and evaporated, yielding 400 mg (73.4%) title compound N-hydroxy-9-[1-(2,2,2-trifluoroacetyl)-piperidin-4-yl]-9H-xanthene-3-carboxamidine, 1m. The crude material was used as such in the next reaction.

### Procedure 25

### 3-(9-Piperidin-4-yl-9H-xanthen-3-yl)-4H-[1,2,4]oxadiazol-5-one, 2m

To a solution of N-hydroxy-9-[1-(2,2,2-trifluoroacetyl)-piperidin-4-yl]-9H-xanthene-3-carboxamidine, 1 m (0.4 g; 0.95 mmol) in dioxane (15 mL) was added 1,1′-carbonyldiimidazole (0.23 g; 1.4 mmol), and the mixture was stirred at 110° C. for 4 h. The mixture was allowed to cool to rt, and evaporated. The residue was dissolved in methanol (8 mL), and treated with 3N NaOH (0.3 mL). The mixture was heated to reflux for 1 h and evaporated. The residue was purified via reverse phase HPLC (eluent: acetonitrile in water containing 0.1% TFA) to yield 30.6 mg (7%) of 3-(9-piperidin-4-yl-9H-xanthen-3-yl)-4H-[1,2,4]oxadiazol-5-one, 2m as a TFA salt. MS m/z (MH⁺) 350.0.

### 3-[9-(1-Furan-3-ylmethyl-piperidin-4-yl)-9H-xanthen-3-yl]-4H-[1,2,4]oxadiazol-5-one, 3m

Using an adaptation of the method described in Procedure 9, substituting the TFA salt of 3-(9-piperidin-4-yl-9H-xanthen-3-yl)-4H-[1,2,4]oxadiazol-5-one, 2m for 9-piperidin-4-yl-9H-xanthene-3-carboxylic acid diethylamide 8a, the title compound 3-(9-piperidin-4-yl-9H-xanthen-3-yl)-4H-[1,2,4]oxadiazol-5-one, 3m was obtained as a TFA salt after reverse phase HPLC purification (eluent: acetonitrile in water containing 0.1% TFA). MS m/z (MH⁺) 430.1.

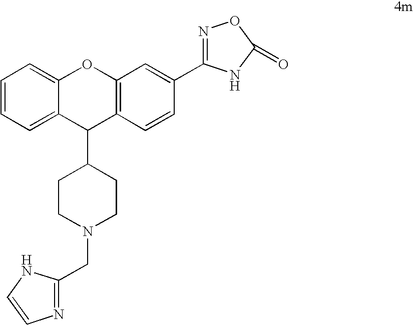

### 3-{9-[1-(1H-Imidazol-2-ylmethyl)-piperidin-4-yl]-9H-xanthen-3-yl}-4H-[1,2,4]oxadiazol-5-one, 4m

Using an adaptation of the method described in Procedure 9, substituting the TFA salt of 3-(9-piperidin-4-yl-9H-xanthen-3-yl)-4H-[1,2,4]oxadiazol-5-one, 2m for 9-piperidin-4-yl-9H-xanthene-3-carboxylic acid diethylamide 8a, and 1H-imidazole-2-carboxaldehyde for 3-furaldehyde, the title compound 3-{9-[1-(1H-imidazol-2-ylmethyl)-piperidin-4-yl]-9H-xanthen-3-yl}-4H-[1,2,4]oxadiazol-5-one, 4m was obtained as a TFA salt after reverse phase HPLC purification (eluent: acetonitrile in water containing 0.1% TFA). MS m/z (MH⁺) 430.1.

### Example N

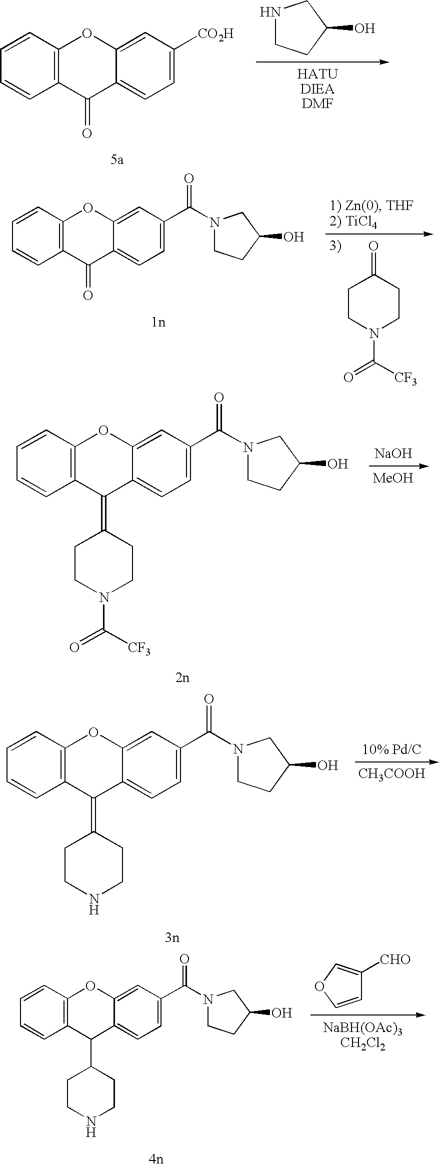

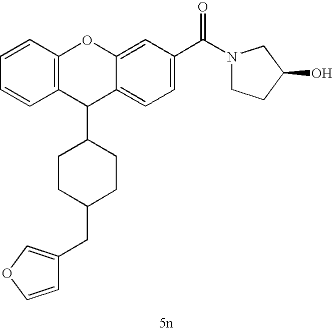

### 3-(3-(S)-Hydroxypyrrolidine-1-carbonyl)-xanthen-9-one, 1n

Using an adaptation of the method described in Procedure 14, substituting 9-oxo-9H-xanthene-3-carboxylic acid, 5a for 5-methoxy-9-oxo-9H-xanthene-3-carboxylic acid, 3c, 3-(S)-hydroxypyrrolidine for N,N-diethylamine, and HATU for HBTU, the title compound 3-(3-(S)-hydroxy-pyrrolidine-1-carbonyl)-xanthen-9-one, 1 n was obtained. MS m/z (MH⁺) 309.9.

### 2,2,2-Trifluoro-1-{4-[3-(3-(S)-hydroxypyrrolidine-1-carbonyl)-xanthen-9-ylidene]-piperidin-1-yl}-ethanone, 2n

Using an adaptation of the method described in Procedure 7, substituting 3-(3-(S)-hydroxy-pyrrolidine-1-carbonyl)-xanthen-9-one, 1n for 9-oxo-9H-xanthene-3-carboxylic acid diethylamide, 6a and 1-(2,2,2-trifluoroacetyl)-piperidin-4-one for 3-oxo-8-aza-bicyclo[3.2.1]-octane-8-carboxylic acid tert-butyl ester, the title compound 2,2,2-trifluoro-1-{4-[3-(3-(S)-hydroxypyrrolidine-1-carbonyl)-xanthen-9-ylidene]-piperidin-1-yl}-ethanone, 3n was obtained. MS m/z (MH⁺) 472.8.

### (3-(S)-Hydroxy-pyrrolidin-1-yl)-(9-piperidin-4-ylidene-9H-xanthen-3-yl)-methanone, 3n

Using an adaptation of the method described in Procedure 17, substituting 2,2,2-trifluoro-1-{4-[3-(3-(S)-hydroxypyrrolidine-1-carbonyl)-xanthen-9-ylidene]-piperidin-1-yl}-ethanone, 3n for 5-hydroxy-9-[1-(2,2,2-trifluoro-acetyl)-piperidin-4-ylidene]-9H-xanthene-3-carboxylic acid ethylamide, 3d, and sodium hydroxide for potassium carbonate, the title compound (3-(S)-hydroxy-pyrrolidin-1-yl)-(9-piperidin-4-ylidene-9H-xanthen-3-yl)-methanone, 3n was obtained.

### (3-(S)-Hydroxy-pyrrolidin-1-yl)-(9-piperidin-4-yl-9H-xanthen-3-yl)-methanone, 4n

Using an adaptation of the method described in Procedure 18, substituting N-[2-(5-hydroxy-9-piperidin-4-ylidene-9H-xanthen-3-yl)-phenyl]-acetamide 3e for 5-hydroxy-9-piperidin-4-ylidene-9H-xanthene-3-carboxylic acid ethylamide 4d, the title compound (3-(S)-hydroxy-pyrrolidin-1-yl)-(9-piperidin-4-yl-9H-xanthen-3-yl)-methanone, 4n was prepared as a TFA salt. MS m/z (MH⁺) 379.2.

### [9-(1-Furan-3-ylmethyl-piperidin-4-yl)-9H-xanthen-3-yl]-(3-(S)-hydroxy-pyrrolidin-1-yl)-methanone, 5n

Using an adaptation of the method described in Procedure 9, substituting the TFA salt of (3-(S)-hydroxy-pyrrolidin-1-yl)-(9-piperidin-4-yl-9H-xanthen-3-yl)methanone, 4n for 9-piperidin-4-yl-9H-xanthene-3-carboxylic acid diethylamide 8a, the title compound [9-(1-furan-3-ylmethyl-piperidin-4-yl)-9H-xanthen-3-yl]-(3-hydroxy-pyrrolidin-1-yl)-methanone, 5n was obtained as a TFA salt after reverse phase HPLC purification (eluent: acetonitrile in water containing 0.1% TFA). MS m/z (MH⁺) 459.1.

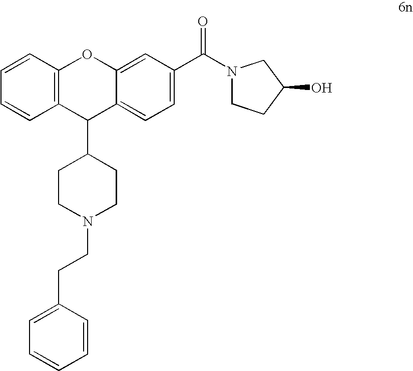

### (3-(S)-Hydroxy-pyrrolidin-1-yl)-[9-(1-phenethyl-piperidin-4-yl)-9H-xanthen-3-yl]-methanone, 6n

Using an adaptation of the method described in Procedure 9, substituting the TFA salt of (3-(S)-hydroxy-pyrrolidin-1-yl)-(9-piperidin-4-yl-9H-xanthen-3-yl)methanone, 4n for 9-piperidin-4-yl-9H-xanthene-3-carboxylic acid diethylamide 8a, and phenyl acetaldehyde for 3-furaldehyde, the title compound (3-(S)hydroxy-pyrrolidin-1-yl)-[9-(1-phenethyl-piperidin-4-yl)-9H-xanthen-3-yl]-methanone, 6n was obtained as a TFA salt after reverse phase HPLC purification (eluent: acetonitrile in water containing 0.1% TFA). MS m/z (MH⁺) 483.2.

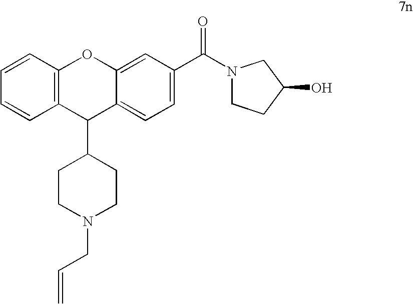

### [9-(1-Allyl-piperidin-4-yl)-9H-xanthen-3-yl]-(3-(S)-hydroxy-pyrrolidin-1-yl)-methanone, 7n

Using an adaptation of the method described in Procedure 20, substituting the TFA salt of (3-(S)-hydroxy-pyrrolidin-1-yl)-(9-piperidin-4-yl-9H-xanthen-3-yl)methanone, 4n for N-[2-(9-piperidin-4-yl-9H-xanthen-3-yl)-phenyl]-acetamide, 2g, the title compound [9-(1-allyl-piperidin-4-yl)-9H-xanthen-3-yl]-(3-(S)hydroxy-pyrrolidin-1-yl)-methanone, 7n was obtained as a TFA salt after reverse phase HPLC purification (eluent: acetonitrile in water containing 0.1% TFA). MS m/z (MH⁺) 419.1.

### Example O

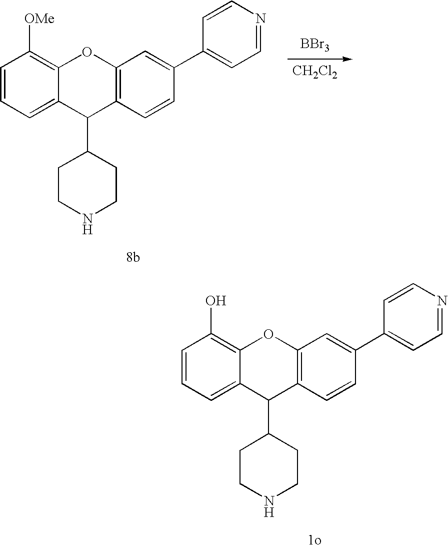

### 9-Piperidin-4-yl-6-pyridin-4-yl-9H-xanthen-4-ol, 1o

Using an adaptation of the method described in Procedure 15, substituting 4-(5-methoxy-9-piperidin-4-yl-9H-xanthen-3-yl)-pyridine, 8b for 5-methoxy-9-piperidin-4-yl-9H-xanthene-3-carboxylic acid diethylamide 6c, the title compound 9-piperidin-4-yl-6-pyridin-4-yl-9H-xanthen-4-ol, 1c as a TFA salt after purification via reverse phase HPLC (eluent: CH₃CN in H₂O containing 0.1% TFA). MS m/z (MH⁺) 359.1.

### Example P

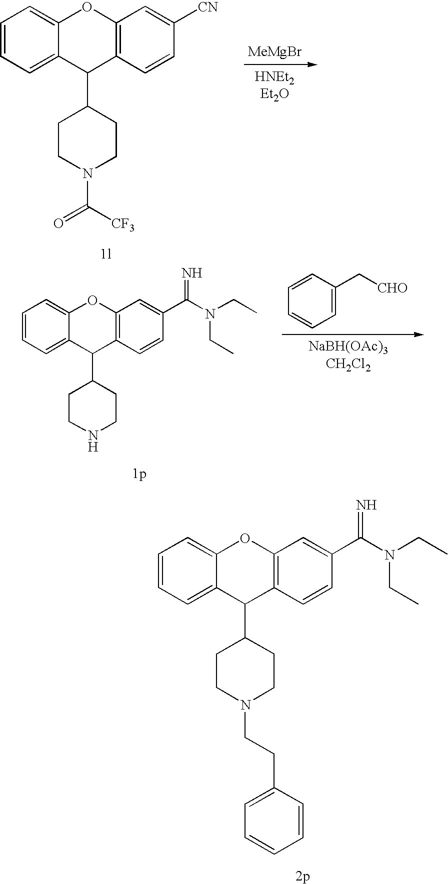

### Procedure 26

### N,N-Diethyl-9-piperidin-4-yl-9H-xanthene-3-carboxamidine, 1p

To a solution of methylmagnesium bromide in diethyl ether (3.0 M, 1.6 mL) under a N₂ atmosphere was added dropwise a solution of diethylamine (0.48 mL, 4.7 mmol) in diethyl ether (2 mL). The mixture was heated to reflux for 30 min and allowed to cool to rt. A solution of 9-[1-(2,2,2-trifluoroacetyl)-piperidin-4-yl]-9H-xanthene-3-carbonitrile, 11 (0.61 g; 1.5 mmol) in diethyl ether (5 mL) was added, and the mixture was heated to reflux for 2 h. Water (10 mL) was added, and the organic layer was separated. The aqueous layer was extracted with methylene chloride and the combined organic layers were dried over MgSO₄, filtered, and evaporated. The residue was purified via reverse phase HPLC (eluent: CH₃CN in H₂O containing 0.1% TFA) to yield 96 mg (10.8%) of title compound N,N-diethyl-9-piperidin-4-yl-9H-xanthene-3-carboxamidine, 1p as a TFA salt. MS m/z (MH⁺) 364.1. ¹H NMR (CD₃OD) δ 7.6 (d, 1H); 7.6 (m, 4H); 7.2 (m, 2H); 4.1 (d, 1H); 3.7 (q, 2H); 3.45 (q, 2H); 3.3-3.4 (m, 2H); 2.7-3.0 (m, 2H); 1.8-2.0 (m, 2H); 1.75 (dd, 1H); 1.4 (t, 3H); 1.2 (t, 3H); 1.6-1.4 (m, 2H).

### N,N-Diethyl-9-(1-phenethyl-piperidin-4-yl)-9H-xanthene-3-carboxamidine, 2p

Using an adaptation of the method described in Procedure 9, substituting the TFA salt of N,N-diethyl-9-piperidin-4-yl-9H-xanthene-3-carboxamidine, 1p for 9-piperidin-4-yl-9H-xanthene-3-carboxylic acid diethylamide, 8a, phenyl acetaldehyde for 3-furaldehyde, sodium triacetoxyborohydride for tetrabutylammonium triacetoxyborohydride, the title compound N,N-diethyl-9-(1-phenethyl-piperidin-4-yl)-9H-xanthene-3-carboxamidine, 2p was obtained as a TFA salt after purification via reverse phase HPLC (eluent: CH₃CN in H₂O containing 0.1% TFA). MS m/z (MH⁺) 468.2.

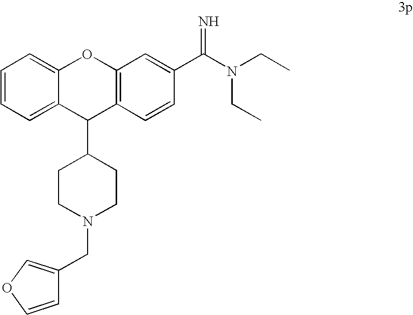

### N,N-Diethyl-9-(1-furan-3-ylmethyl-piperidin-4-yl)-9H-xanthene-3-carboxamidine, 3p

Using an adaptation of the method described in Procedure 9, substituting the TFA salt of N,N-diethyl-9-piperidin-4-yl-9H-xanthene-3-carboxamidine, 1p for 9-piperidin-4-yl-9H-xanthene-3-carboxylic acid diethylamide, 8a, and sodium triacetoxyborohydride for tetrabutylammonium triacetoxyborohydride, the title compound N,N-diethyl-9-(1-furan-3-ylmethyl-piperidin-4-yl)-9H-xanthene-3-carboxamidine, 3p was obtained as a TFA salt after purification via reverse phase HPLC (eluent: CH₃CN in H₂O containing 0.1% TFA). MS m/z (MH⁺) 444.1.

### Example Q

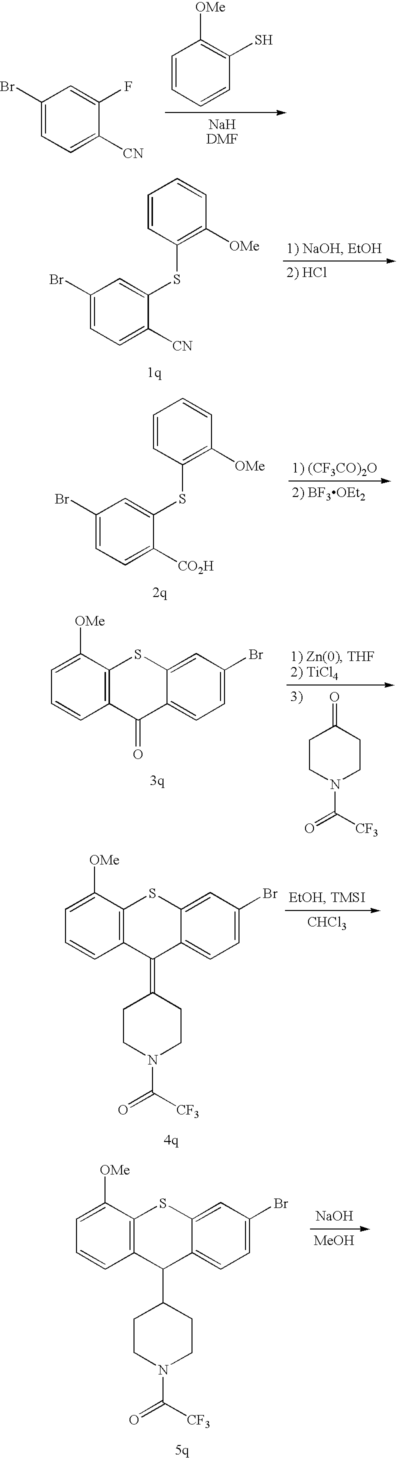

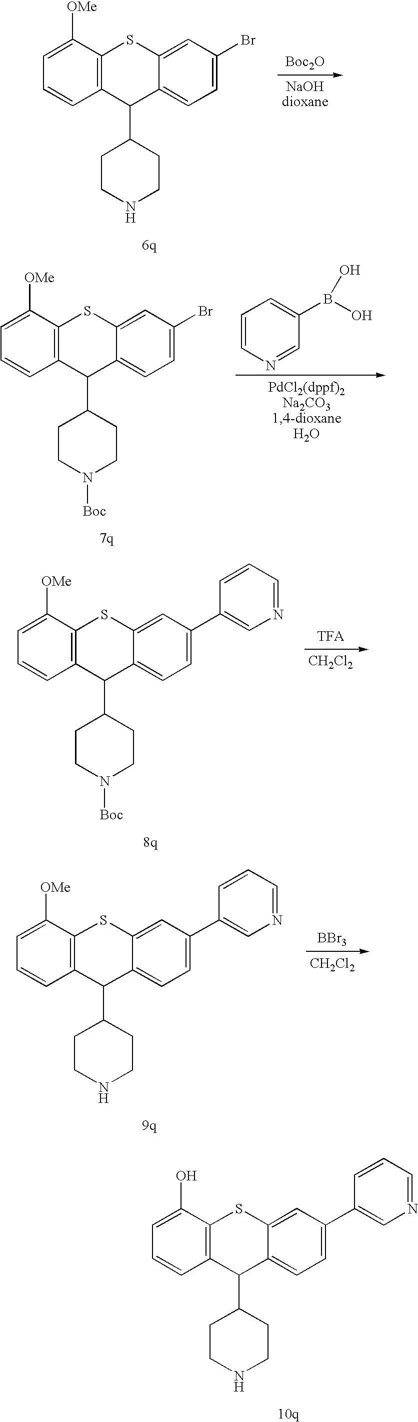

### 4-Bromo-2-(2-methoxyphenylsulfanyl)-benzonitrile, 1q

Using an adaptation of the method described in Procedure 1, substituting 2-methoxythiophenol for phenol, the title compound 4-bromo-2-(2-methoxyphenylsulfanyl)-benzonitrile, 1q was obtained.

### 4-Bromo-2-(2-methoxyphenylsulfanyl)-benzoic acid, 2q

Using an adaptation of the method described in Procedure 2, substituting 4-bromo-2-(2-methoxyphenylsulfanyl)-benzonitrile, 1 q for 4-bromo-2-phenoxybenzonitrile, 1a, the title compound 4-bromo-2-(2-methoxyphenylsulfanyl)-benzoic acid, 2q was obtained.

### 3-Bromo-5-methoxythioxanthen-9-one, 3q

Using an adaptation of the method described in Procedure 3, substituting 4-bromo-2-(2-methoxyphenylsulfanyl)-benzoic acid, 2q for 4-bromo-2-phenoxybenzoic acid, 2a, the title compound 3-bromo-5-methoxythioxanthen-9-one, 3q was obtained.

### 1-[4-(3-Bromo-5-methoxythioxanthen-9-ylidene)-piperidin-1-yl]-2,2,2-trifluoroethanone, 4q

Using an adaptation of the method described in Procedure 4, substituting 3-bromo-5-methoxythioxanthen-9-one, 3q for 9-oxo-9H-xanthene-3-carboxylic acid diethylamide, 6a, and 1-(2,2,2-trifluoroacetyl)-piperidin-4-one for 4-oxopiperidine-1-carboxylic acid tert-butyl ester, the title compound 1-[4-(3-bromo-5-methoxythioxanthen-9-ylidene)-piperidin-1-yl]-2,2,2-trifluoroethanone, 4q was obtained. MS m/z (MH⁺) 485.7.

### 1-[4-(3-Bromo-5-methoxy-9H-thioxanthen-9-yl)-piperidin-1-yl]-2,2,2-trifluoroethanone, 5q

Using an adaptation of the method described in Procedure 8, substituting 1-[4-(3-bromo-5-methoxythioxanthen-9-ylidene)-piperidin-1-yl]-2,2,2-trifluoro-ethanone, 4q for 9-piperidin-4-ylidene-9H-xanthene-3-carboxylic acid diethylamide, 8a, the title compound 1-[4-(3-bromo-5-methoxy-9H-thioxanthen-9-yl)-piperidin-1-yl]-2,2,2-trifluoroethanone, 5q was obtained. MS m/z (MH⁺) 487.6.

### 4-(3-Bromo-5-methoxy-9H-thioxanthen-9-yl)-piperidine, 6q

Using an adaptation of the method described in Procedure 10, substituting 1-[4-(3-bromo-5-methoxy-9H-thioxanthen-9-yl)-piperidin-1-yl]-2,2,2-trifluoroethanone, 5q for 1-[4-(3-bromo-5-methoxy-9H-xanthen-9-yl)-piperidin-1-yl]-2,2,2-trifluoro-ethanone, 5b, the title

### Procedure 27

### 4-(3-Bromo-5-methoxy-9H-thioxanthen-9-yl)-piperidine-1-carboxylic acid tert-butyl ester, 7q

To a solution of 4-(3-bromo-5-methoxy-9H-thioxanthen-9-yl)-piperidine, 6q (3.0 g, 7.7 mmol) in dioxane (40 mL) were added Boc anhydride (1.6 g, 8.5 mmol) and a 3N NaOH solution (8 mL). The mixture was allowed to stir for at rt for 2 h, and the solvent was evaporated. The residue was purified via flash column chromatography, yielding 1.4 g (37.2%) of title compound 4-(3-bromo-5-methoxy-9H-thioxanthen-9-yl)-piperidine-1-carboxylic acid tert-butyl ester, 7q. MS m/z (MH⁺) 490.2.

### 4-(5-Methoxy-3-pyridin-3-yl-9H-thioxanthen-9-yl)-piperidine-1-carboxylic acid tert-butyl ester, 8q

Using an adaptation of the method described in Procedure 11, substituting 4-(3-bromo-5-methoxy-9H-thioxanthen-9-yl)-piperidine-1-carboxylic acid tert-butyl ester, 7q for of 4-(3-bromo-5-methoxy-9H-xanthen-9-yl)-piperidine, 6b, crude title compound 4-(5-methoxy-3-pyridin-3-yl-9H-thioxanthen-9-yl)-piperidine-1-carboxylic acid tert-butyl ester, 8q was obtained. The compound was used as such for the next reaction.

### Procedure 28

### 3-(5-Methoxy-9-piperidin-4-yl-9H-thioxanthen-3-yl)-pyridine, 9q

To a solution of crude 4-(5-methoxy-3-pyridin-3-yl-9H-thioxanthen-9-yl)-piperidine-1-carboxylic acid tert-butyl ester, 8q (233 mg, 0.6 mmol) in methylene chloride (20 mL) was added TFA (2 mL), and the mixture was allowed to stir for 1 h at rt. The mixture was evaporated and the residue was purified via reverse phase HPLC (eluent: CH₃CN in water containing 0.1% TFA) to yield 110 mg (35.5%) of title compound 3-(5-methoxy-9-piperidin-4-yl-9H-thioxanthen-3-yl)-pyridine, 9q as a TFA salt. MS m/z (MH⁺) 389.0.

### 9-Piperidin-4-yl-6-pyridin-3-yl-9H-thioxanthen-4-ol, 10q

Using an adaptation of the method described in Procedure 15, substituting the TFA salt of 3-(5-methoxy-9-piperidin-4-yl-9H-thioxanthen-3-yl)-pyridine, 9q for the TFA salt of 5-methoxy-9-piperidin-4-yl-9H-xanthene-3-carboxylic acid diethylamide, 6c, the title compound 9-piperidin-4-yl-6-pyridin-3-yl-9H-thioxanthen-4-ol, 10q was obtained as a TFA salt after reverse phase HPLC (eluent: CH₃CN in water containing 0.1% TFA). MS m/z (MH⁺) 375.0.

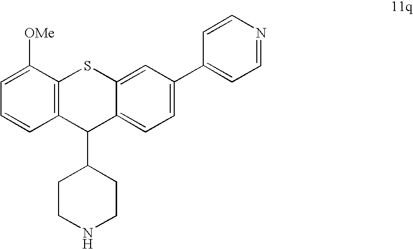

### 4-(5-Methoxy-9-piperidin-4-yl-9H-thioxanthen-3-yl)-pyridine, 11q

Using an adaptation of the method described in Procedures 11 and 28, substituting 4-(3-bromo-5-methoxy-9H-thioxanthen-9-yl)-piperidine-1-carboxylic acid tert-butyl ester, 7q for of 4-(3-bromo-5-methoxy-9H-xanthen-9-yl)piperidine, 6b, and 4-pyridyl boronic acid for 3-pyridyl boronic acid in Procedure 11, title compound 4-(5-methoxy-9-piperidin-4-yl-9H-thioxanthen-3-yl)-pyridine, 11q was obtained as a TFA salt. MS m/z (MH⁺) 389.1.

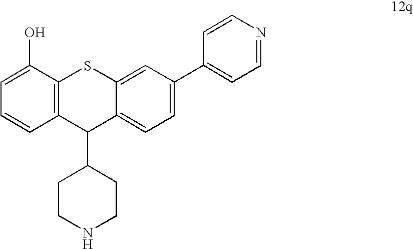

### 9-Piperidin-4-yl-6-pyridin-4-yl-9H-thioxanthen-4-ol, 12q

Using an adaptation of the method described in Procedure 15, substituting the TFA salt of 4-(5-methoxy-9-piperidin-4-yl-9H-thioxanthen-3-yl)-pyridine, 11q for the TFA salt of 5-methoxy-9-piperidin-4-yl-9H-xanthene-3-carboxylic acid diethylamide, 6c, the title compound 9-piperidin-4-yl-6-pyridin-4-yl-9H-thioxanthen-4-ol, 12q was obtained as a TFA salt after reverse phase HPLC (eluent: CH₃CN in water containing 0.1% TFA). MS m/z (MH⁺) 375.0.

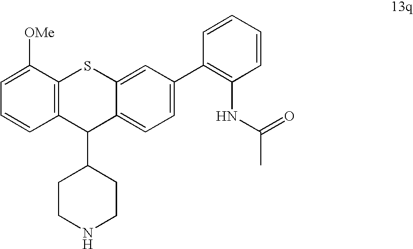

### N-[2-(5-Methoxy-9-piperidin-4-yl-9H-thioxanthen-3-yl)-phenyl]-acetamide, 13q

Using an adaptation of the method described in Procedures 11 and 28, substituting 4-(3-bromo-5-methoxy-9H-thioxanthen-9-yl)-piperidine-1-carboxylic acid tert-butyl ester, 7q for of 4-(3-bromo-5-methoxy-9H-xanthen-9-yl)piperidine, 6b, and 2-acetylaminophenyl boronic acid for 3-pyridyl boronic acid in Procedure 11, title compound N-[2-(5-methoxy-9-piperidin-4-yl-9H-thioxanthen-3-yl)-phenyl]-acetamide, 13q was obtained as a TFA salt. MS m/z (MH⁺) 445.2.

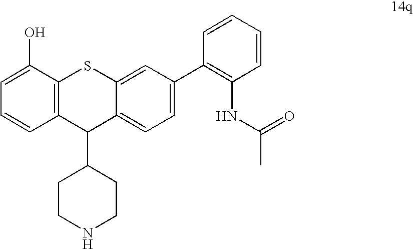

### N-[2-(5-Hydroxy-9-piperidin-4-yl-9H-thioxanthen-3-yl)-phenyl]-acetamide, 14q

Using an adaptation of the method described in Procedure 15, substituting the TFA salt of N-[2-(5-methoxy-9-piperidin-4-yl-9H-thioxanthen-3-yl)-phenyl]-acetamide, 13q for the TFA salt of 5-methoxy-9-piperidin-4-yl-9H-xanthene-3-carboxylic acid diethylamide, 6c, the title compound N-[2-(5-hydroxy-9-piperidin-4-yl-9H-thioxanthen-3-yl)-phenyl]-acetamide, 14q was obtained as a TFA salt after reverse phase HPLC (eluent: CH₃CN in water containing 0.1% TFA). MS m/z (MH⁺) 431.1.

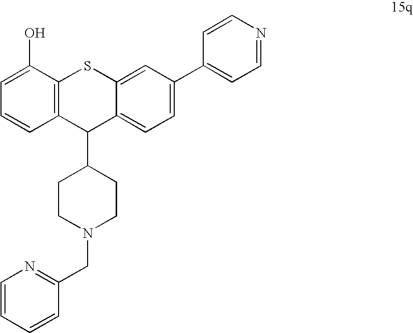

### 6-Pyridin-4-yl-9-(1-pyridin-2-ylmethyl-piperidin-4-yl)-9H-thioxanthen-4-ol, 15q

Using an adaptation of the method described in Procedure 9, substituting the TFA salt of 9-piperidin-4-yl-6-pyridin-4-yl-9H-thioxanthen-4-ol, 12q for 9-piperidin-4-yl-9H-xanthene-3-carboxylic acid diethylamide, 8a, and sodium triacetoxyborohydride for tetrabutylammonium triacetoxyborohydride, the title compound 6-pyridin-4-yl-9-(1-pyridin-2-ylmethyl-piperidin-4-yl)-9H-thioxanthen-4-ol, 15q was obtained as a TFA salt after purification via reverse phase HPLC (eluent: CH₃CN in H₂O containing 0.1% TFA). MS m/z (MH⁺) 466.0.

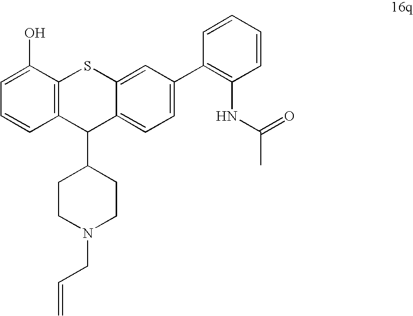

### N-{2-[9-(1-Allyl-piperidin-4-yl)-5-hydroxy-9H-thioxanthen-3-yl]-phenyl}-acetamide, 16q

Using an adaptation of the method described in Procedure 20, substituting the TFA salt of N-[2-(5-hydroxy-9-piperidin-4-yl-9H-thioxanthen-3-yl)-phenyl]-acetamide, 14q for N-[2-(9-piperidin-4-yl-9H-xanthen-3-yl)-phenyl]-acetamide, 2g, the title compound N-{2-[9-(1-allyl-piperidin-4-yl)-5-hydroxy-9H-thioxanthen-3-yl]-phenyl}-acetamide, 16q was obtained as a TFA salt after purification via reverse phase HPLC (eluent: CH₃CN in H₂O containing 0.1% TFA). MS m/z (MH⁺) 455.1.

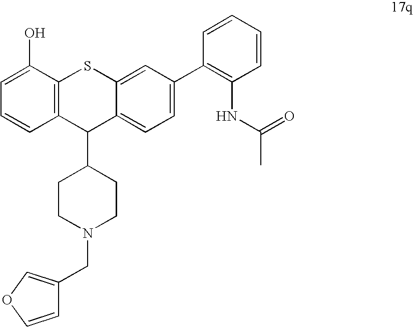

### N-{2-[9-(1-Furan-3-ylmethyl-piperidin-4-yl)-5-hydroxy-9H-thioxanthen-3-yl]-phenyl}-acetamide, 17q

Using an adaptation of the method described in Procedure 9, substituting the TFA salt of N-[2-(5-hydroxy-9-piperidin-4-yl-9H-thioxanthen-3-yl)-phenyl]-acetamide, 14q for 9-piperidin-4-yl-9H-xanthene-3-carboxylic acid diethylamide, 8a, and sodium triacetoxyborohydride for tetrabutylammonium triacetoxyborohydride, the title compound N-{2-[9-(1-furan-3-ylmethyl-piperidin-4-yl)-5-hydroxy-9H-thioxanthen-3-yl]-phenyl}-acetamide, 17q was obtained as a TFA salt after purification via reverse phase HPLC (eluent: CH₃CN in H₂O containing 0.1% TFA). MS m/z (MH⁺) 511.1.

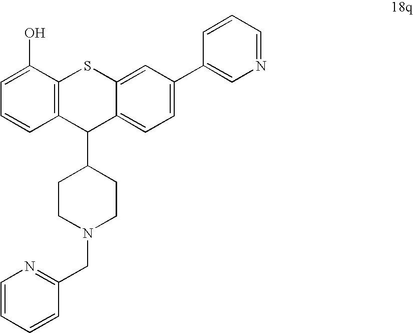

### 6-Pyridin-3-yl-9-(1-pyridin-2-ylmethyl-piperidin-4-yl)-9H-thioxanthen-4-ol, 18q

Using an adaptation of the method described in Procedure 9, substituting the TFA salt of 9-piperidin-4-yl-6-pyridin-3-yl-9H-thioxanthen-4-ol, 10q for 9-piperidin-4-yl-9H-xanthene-3-carboxylic acid diethylamide, 8a, 2-pyridyl carboxaldehyde for 3-furaldehyde, and sodium triacetoxyborohydride for tetrabutylammonium triacetoxyborohydride, the title compound 6-pyridin-3-yl-9-(1-pyridin-2-ylmethyl-piperidin-4-yl)-9H-thioxanthen-4-ol, 18q was obtained as a TFA salt after purification via reverse phase HPLC (eluent: CH₃CN in H₂O containing 0.1% TFA). MS m/z (MH⁺) 466.1.

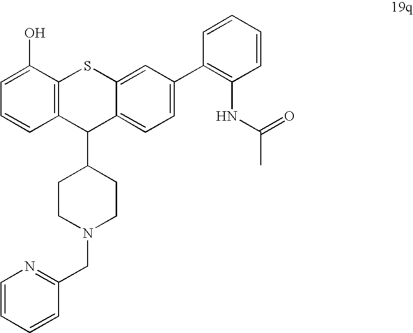

### N-{2-[5-Hydroxy-9-(1-pyridin-2-ylmethyl-piperidin-4-yl)-9H-thioxanthen-3-yl]-phenyl}-acetamide, 19q

Using an adaptation of the method described in Procedure 9, substituting the TFA salt of N-[2-(5-hydroxy-9-piperidin-4-yl-9H-thioxanthen-3-yl)-phenyl]-acetamide, 14q for 9-piperidin-4-yl-9H-xanthene-3-carboxylic acid diethylamide, 8a, sodium triacetoxyborohydride for tetrabutylammonium triacetoxyborohydride, and 2-pyridyl carboxaldehyde for 3-furaldehyde, the title compound N-{2-[5-hydroxy-9-(1-pyridin-2-ylmethyl-piperidin-4-yl)-9H-thioxanthen-3-yl]-phenyl}-acetamide, 19q was obtained as a TFA salt after purification via reverse phase HPLC (eluent: CH₃CN in H₂O containing 0.1% TFA). MS m/z (MH⁺) 522.1.

### Example R

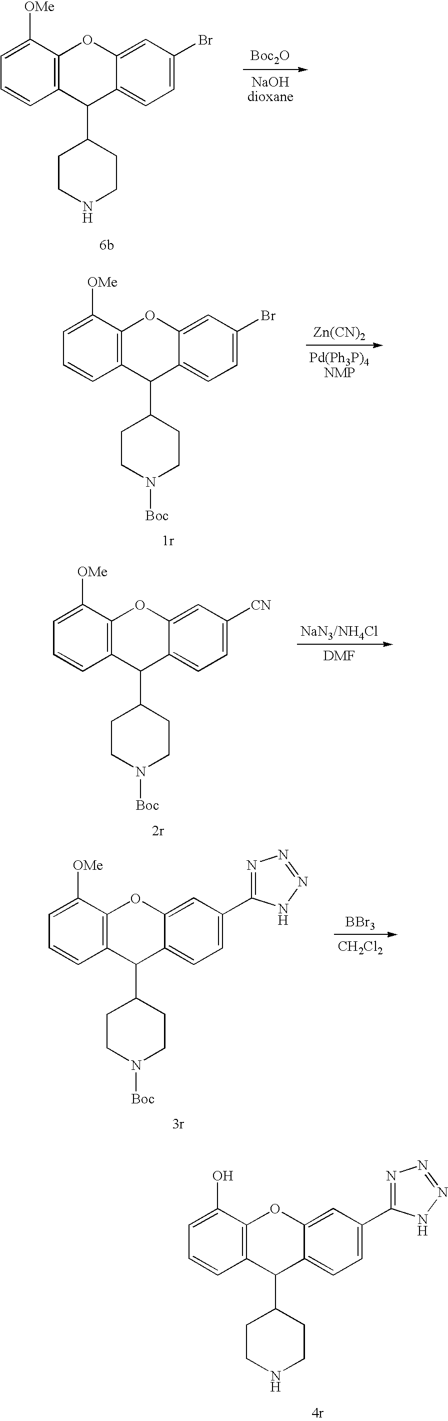

### 4-(3-Bromo-5-methoxy-9H-xanthen-9-yl)-piperidine-1-carboxylic acid tert-butyl ester, 1r

Using an adaptation of the method described in Procedure 26, substituting 4-(3-bromo-5-methoxy-9H-xanthen-9-yl)-piperidine, 6b for 4-(3-bromo-5-methoxy-9H-thioxanthen-9-yl)-piperidine, 6q, the title compound 4-(3-bromo-5-methoxy-9H-xanthen-9-yl)-piperidine-1-carboxylic acid tert-butyl ester, 1 r was obtained. MS m/z (MH⁺) 474.9.

### 4-(3-Cyano-5-methoxy-9H-xanthen-9-yl)-piperidine-1-carboxylic acid tert-butyl ester, 2r

Using an adaptation of the method described in Procedure 21, substituting 4-(3-bromo-5-methoxy-9H-xanthen-9-yl)-piperidine-1-carboxylic acid tert-butyl ester, 1r for 1-[4-(3-bromo-9H-xanthen-9-yl)-piperidin-1-yl]-2,2,2-trifluoroethanone, 2f, the title compound 4-(3-cyano-5-methoxy-9H-xanthen-9-yl)piperidine-1-carboxylic acid tert-butyl ester, 2r was obtained. MS m/z (MH⁺) 420.9.

### 4-[5-Methoxy-3-(1H-tetrazol-5-yl)-9H-xanthen-9-yl]-piperidine-1-carboxylic acid tert-butyl ester, 3r

Using an adaptation of the method described in Procedure 22, substituting 4-(3-cyano-5-methoxy-9H-xanthen-9-yl)-piperidine-1-carboxylic acid tert-butyl ester, 2r for 9-[1-(2,2,2-trifluoroacetyl)-piperidin-4-yl]-9H-xanthene-3-carbonitrile, 1l, the title compound 4-[5-methoxy-3-(1H-tetrazol-5-yl)-9H-xanthen-9-yl]-piperidine-1-carboxylic acid tert-butyl ester, 3r was obtained. MS m/z (MH⁺) 464.1.

### 9-Piperidin-4-yl-6-(1H-tetrazol-5-yl)-9H-xanthen-4-ol, 4r

Using an adaptation of the method described in Procedure 15, substituting 4-[5-methoxy-3-(1H-tetrazol-5-yl)-9H-xanthen-9-yl]-piperidine-1-carboxylic acid tert-butyl ester, 3r for the TFA salt of 5-methoxy-9-piperidin-4-yl-9H-xanthene-3-carboxylic acid diethylamide, 6c, the title compound 4-[5-methoxy-3-(1H-tetrazol-5-yl)-9H-xanthen-9-yl]-piperidine-1-carboxylic acid tert-butyl ester, 3r was obtained as a TFA salt after purification via reverse phase HPLC (eluent: CH₃CN in water containing 0.1% TFA). MS m/z (MH⁺) 350.1.

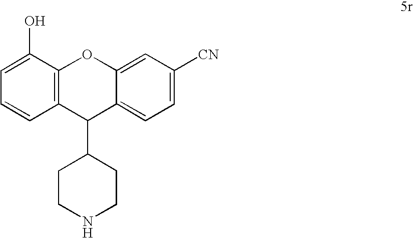

### 5-Hydroxy-9-piperidin-4-yl-9H-xanthene-3-carbonitrile, 5r

Using an adaptation of the method described in Procedure 15, substituting 4-(3-cyano-5-methoxy-9H-xanthen-9-yl)-piperidine-1-carboxylic acid tert-butyl ester, 2r for the TFA salt of 5-methoxy-9-piperidin-4-yl-9H-xanthene-3-carboxylic acid diethylamide, 6c, the title compound 5-hydroxy-9-piperidin-4-yl-9H-xanthene-3-carbonitrile, 5r was obtained as a TFA salt after purification via reverse phase HPLC (eluent: CH₃CN in water containing 0.1% TFA). MS m/z (MH⁺) 307.0.

### Example S

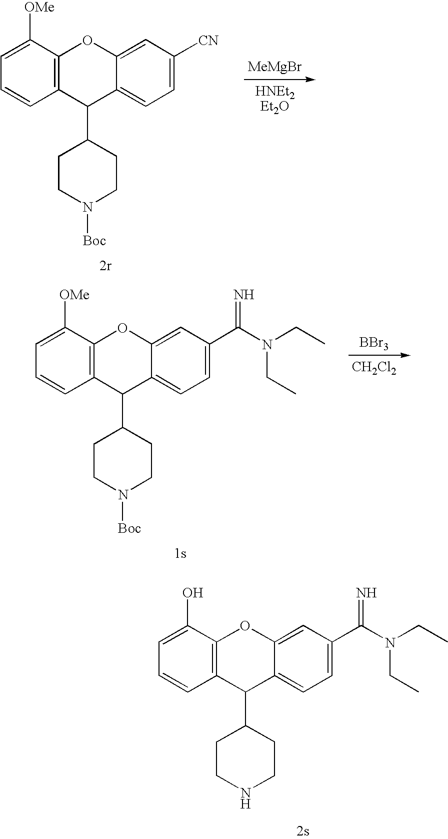

### 4-[3-(N,N-Diethyl-carbamimidoyl)-5-methoxy-9H-xanthen-9-yl]-piperidine-1-carboxylic acid tert-butyl ester, 1s

Using an adaptation of the method described in Procedure 26, substituting 4-(3-cyano-5-methoxy-9H-xanthen-9-yl)-piperidine-1-carboxylic acid tert-butyl ester, 2r for 9-[1-(2,2,2-trifluoroacetyl)-piperidin-4-yl]-9H-xanthene-3-carbonitrile, 1l, the title compound 4-[3-(N,N-diethyl-carbamimidoyl)-5-methoxy-9H-xanthen-9-yl]-piperidine-1-carboxylic acid tert-butyl ester, 1s was obtained as a TFA salt after purification via reverse phase HPLC (eluent: CH₃CN in water containing 0.1% TFA). MS m/z (MH⁺) 494 (and 393.9, loss of Boc group).

### N,N-Diethyl-5-hydroxy-9-piperidin-4-yl-9H-xanthene-3-carboxamidine, 2s

Using an adaptation of the method described in Procedure 15, substituting the TFA salt of 4-[3-(N,N-diethyl-carbamimidoyl)-5-methoxy-9H-xanthen-9-yl]-piperidine-1-carboxylic acid tert-butyl ester, 1s for the TFA salt of 5-methoxy-9-piperidin-4-yl-9H-xanthene-3-carboxylic acid diethylamide, 6c, the title compound N,N-diethyl-5-hydroxy-9-piperidin-4-yl-9H-xanthene-3-carboxamidine, 2s was obtained as a TFA salt after purification via reverse phase HPLC (eluent: CH₃CN in water containing 0.1% TFA). MS m/z (MH⁺) 380.1.

### Example T

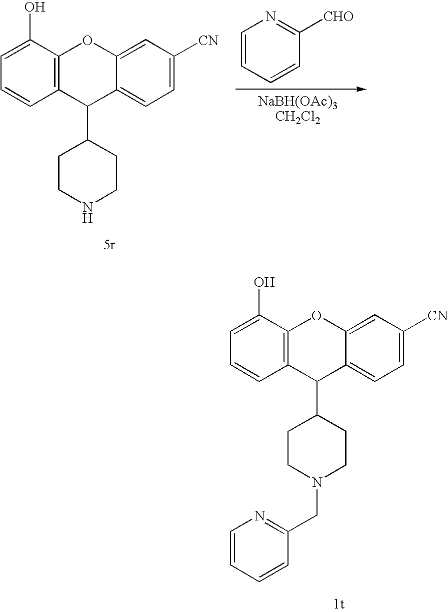

### 5-Hydroxy-9-(1-pyridin-2-ylmethyl-piperidin-4-yl)-9H-xanthene-3-carbonitrile, 1t

Using an adaptation of the method described in Procedure 9, substituting the TFA salt of 5-hydroxy-9-piperidin-4-yl-9H-xanthene-3-carbonitrile, 5r for 9-piperidin-4-yl-9H-xanthene-3-carboxylic acid diethylamide, 8a, and sodium triacetoxyborohydride for tetrabutylammonium triacetoxyborohydride, the title compound 5-hydroxy-9-(1-pyridin-2-ylmethyl-piperidin-4-yl)-9H-xanthene-3-carbonitrile, 1t was obtained as a TFA salt after purification via reverse phase HPLC (eluent: CH₃CN in H₂O containing 0.1% TFA). MS m/z (MH⁺) 398.1.

### Example U

The (+) and (−) enantiomers of 1-[4-(3-bromo-9H-xanthen-9-yl)-piperidin-1-yl]-2,2,2-trifluoro-ethanone, 2f were separated on an analytical Chiralpak AD column (25×0.46 cm) and using hexane:EtOH:MeOH (80:15:5) as eluent. The analytes were monitored using a wavelength of 220 nm.

### Biological Examples

### Rat Brain Delta Opioid Receptor Binding Assay

Procedure: Male, Wistar rats (150-250 g, VAF, Charles River, Kingston, N.Y.) were killed by CO₂, and their brains were removed and placed immediately in ice cold Tris HCl buffer (50 mM, pH 7.4). The forebrains were separated from the remainder of the brain by a coronal transection, beginning dorsally at the colliculi and passing ventrally through the midbrain-pontine junction. After dissection, the forebrains were homogenized in Tris buffer in a Teflon®-glass homogenizer. The homogenate was diluted to a concentration of 1 g of forebrain tissue per 80 mL Tris and centrifuged at 39,000×g for 10 min. The pellet was resuspended in the same volume of Tris buffer containing 5 mM MgCl₂ with several brief pulses from a Polytron homogenizer. This particulate preparation was used for the delta opioid binding assays. Following incubation with the delta selective peptide ligand ˜4 nM [³H]DPDPE or 0.15 nM [³H]naltrindole at 25° C. for 2.5 h in a 96-well plate with total volume of 1 mL, the plate contents were filtered through Wallac filtermat B sheets on a Tomtec 96-well harvester. The filters were rinsed three times with 2 mL of 10 mM HEPES (pH 7.4), and dried in a 650 W microwave oven for 1.75 min twice. To each sample area 2×50 μL of Betaplate Scint scintillation fluid (LKB) was added and the radioactivity was quantified on a LKB (Wallac) 1205 BetaPlate liquid scintillation counter.

Analysis: The data from the scintillation counter was used to calculate either the % inhibition compared to control binding (when only a single concentration of test compound was evaluated) or a Kᵢ value (when a range of concentrations was tested). Percent inhibition was calculated as: [(total dpm-test compound dpm)/(total dpm-nonspecific dpm)]*100. Kd and Ki values were calculated using GraphPad PRISM data analysis program. The data obtained are shown in Table 1, below.

### Rat Brain Mu Opioid Receptor Binding Assay

Procedure: Male, Wistar rats (150-250 g, VAF, Charles River, Kingston, N.Y.) were killed by CO₂, and their brains were removed and placed immediately in ice cold Tris HCl buffer (50 mM, pH 7.4). The forebrains were separated from the remainder of the brain by a coronal transection, beginning dorsally at the colliculi and passing ventrally through the midbrain-pontine junction. After dissection, the forebrains were homogenized in Tris buffer in a Teflon®-glass homogenizer. The homogenate was diluted to a concentration of 1 g of forebrain tissue per 80 mL Tris and centrifuged at 39,000×g for 10 min. The pellet was resuspended in the same volume of Tris buffer containing 5 mM MgCl₂ with several brief pulses from a Polytron homogenizer. This particulate preparation was used for the mu opioid binding assays. Following incubation with the mu selective peptide ligand, ˜0.8 nM [³H]DAMGO, at 25° C. for 2.5 h in a 96-well plate with total assay volume of 1 mL, the plate contents were filtered through Wallac filtermat B sheets on a Tomtec 96-well harvester. The filters were rinsed three times with 2 mL of 10 mM HEPES (pH 7.4), and dried in a 650 W microwave oven for 1.75 min twice. To each sample area 2×40 μL of Betaplate Scint scintillation fluid (LKB) was added and the radioactivity was quantified on a LKB (Wallac) 1205 BetaPlate liquid scintillation counter.

Analysis: The data from the scintillation counter was used to calculate either the % inhibition compared to control binding (when only a single concentration of test compound was evaluated) or a Kᵢ value (when a range of concentrations was tested). Percent inhibition was calculated as: [(total dpm-test compound dpm)/(total dpm-nonspecific dpm)]*100. Kd and Ki values were calculated using GraphPad PRISM data analysis program. The data obtained are shown in Table 1, below.

### [³⁵S]GTPγS Binding Assay in NG108-15 Cell Membranes

### Delta Opioid

Methods: NG108-15 cell membranes were purchased from Applied Cell Sciences (Rockville, Md.). 8 mg/mL of membrane protein was suspended in 10 mM TRIS-HCl pH 7.2, 2 mM EDTA, 10% sucrose. Membranes were maintained at 4-8° C. A 1 mL volume of membranes was added into 10 mL cold binding assay buffer. The assay buffer contained 50 mM Tris, pH 7.6, 5 mM MgCl₂, 100 mM NaCl, 1 mM DTT and 1 mM EGTA. The membrane suspension was homogenized twice with a Polytron, and centrifuged at 3000 rpm for 10 min. The supernatant was then centrifuged at 18,000 rpm for 20 min. Ten mL assay buffer was added into the pellet containing tube. The pellet and buffer were mixed with a Polytron.

Incubation procedure: The pellet membranes (75 μg/mL) were preincubated with SPA (10 mg/mL) at 25° C. for 45 min in the assay buffer. The SPA (5 mg/mL) coupled with membranes (37.5 μg/mL) were then incubated with 0.1 nM [³⁵S]GTPγS in the same Tris buffer containing 100 μM GDP in a total volume of 200 μL. Increasing concentrations of receptor agonists were used to stimulate [³⁵S]-GTP□S binding. The basal binding was tested in the absence of agonists and non-specific binding was tested in the presence of 10 μM unlabeled GTPγS. The data were analyzed on a Packard Top Count.

### Data

% of Basal=(stimulated−non specific)*100/(basal−non specific).

EC₅₀ value values were calculated using GraphPad Prism.
The data obtained are shown in Table 1, below.

### [³⁵S]GTPγS Binding Assays in CHO-hMOR Cell Membranes

Methods: CHO-hMOR cell membranes can be purchased from Receptor Biology, Inc. (Baltimore, Md.). About 10 mg/mL of membrane protein can be suspended in 10 mM TRIS-HCl pH 7.2, 2 mM EDTA, 10% sucrose, and the suspension kept on ice. A 1 mL volume of membranes can be added to 15 mL cold binding assay buffer containing 50 mM HEPES, pH 7.6, 5 mM MgCl₂, 100 mM NaCl, 1 mM DTT and 1 mM EDTA. The membrane suspension can be homogenized with a Polytron and centrifuged at 3,000 rpm for 10 min. The supernatant can then be centrifuged at 18,000 rpm for 20 min. The pellet can be resuspended in 10 mL assay buffer with a Polytron. The membranes can be preincubated with wheat germ agglutinin coated SPA beads (Amersham) at 25° C. for 45 min in the assay buffer. The SPA bead (5 mg/mL) coupled membranes (10 μg/mL) can be then incubated with 0.5 nM [³⁵S]GTPγS in the assay buffer. The basal binding can be that taking place in the absence of added test compound; this unmodulated binding can be considered as 100%, with agonist stimulated binding rising to levels significantly above this value. A range of concentrations of receptor agonist can be used to stimulate [³⁵S]GTPγS binding. Both basal and non-specific binding can be tested in the absence of agonist; non-specific binding determination included 10 μM unlabeled GTPγS.

Compounds can be tested for function as antagonists by evaluating their potential to inhibit agonist-stimulated GTPγS binding. Radioactivity can be quantified on a Packard TopCount. The following parameters can be calculated:

$$\% stimulation = \frac{( test compound cpm - non \text{-} specific cpm )}{( basal cpm - non \text{-} specific cpm ) .} \times 100$$

$$\% inhibition = \frac{( \begin{matrix}\% stimulation by 1 µM DAMGO - \\ \% stimulation by test compound\end{matrix} )}{( \% stimulation by 1 µM DAMGO - 100 )} \times 100$$

EC₅₀ values can be calculated using GraphPad Prism.

TABLE 1

| | | | | delta | |
|---|---|---|---|---|---|
| | | | mu | GTPγS | delta |
| | | delta | (Ki, | EC50 | GTPγS |
| Compound | Ex. # | (Ki, nM) | nM) | (nM) | Rel Eff |
| 5-Hydroxy-9-piperidin-4-yl-9H- | 7c | 2.0 | 4234 | 18.7 | 0.51 |
| xanthene-3-carboxylic acid | | | | | |
| diethylamide | | | | | |
| 9-Piperidin-4-yl-9H-xanthene-3- | 8a | 15 | 8792.5 | 266 | |
| carboxylic acid diethylamide | | | | | |
| N,N-Diethyl-3-(5-hydroxy-9-piperidin- | 3j | 37.8 | 535 | 342 | 0.63 |
| 4-yl-9H-xanthen-3-yl)-benzamide | | | | | |
| N-[2-(5-Hydroxy-9-piperidin-4-yl-9H- | 4e | 38.1 | 289 | | |
| xanthen-3-yl)-phenyl]-acetamide | | | | | |
| N,N-Diethyl-9-piperidin-4-yl-9H- | 1p | 51.12 | >10000 | | |
| xanthene-3-carboxamidine | | | | | |
| 9-(1-Benzyl-piperidin-4-yl)-9H- | 10a | 72 | 5372.0 | | |
| xanthene-3-carboxylic acid | | | | | |
| diethylamide | | | | | |
| N-{2-[9-(1-Furan-3-ylmethyl- | 3g | 100 | 4440 | | |
| piperidin-4-yl)-9H-xanthen-3-yl]- | | | | | |
| phenyl}-acetamide | | | | | |
| 9-[1-(1H-Imidazol-2-ylmethyl)- | 13a | 106 | 1702 | | |
| piperidin-4-yl]-9H-xanthene-3- | | | | | |
| carboxylic acid diethylamide | | | | | |
| 9-(1-Furan-3-ylmethyl-piperidin-4-yl)- | 9a | 141 | 51041 | | |
| 9H-xanthene-3-carboxylic acid | | | | | |
| diethylamide | | | | | |
| 3-(9-Piperidin-4-yl-9H-xanthen-3-yl)- | 4f | 161 | 1002 | | |
| pyridine | | | | | |
| N-{2-[9-(1-Pyridin-2-ylmethyl- | 4g | 167 | 6976 | | |
| piperidin-4-yl)-9H-xanthen-3-yl]- | | | | | |
| phenyl}-acetamide | | | | | |
| [9-(1-Furan-3-ylmethyl-piperidin-4- | 5n | 176.5 | >10000 | | |
| yl)-9H-xanthen-3-yl]-(3-(S)-hydroxy- | | | | | |
| pyrrolidin-1-yl)-methanone | | | | | |
| 5-Hydroxy-9-piperidin-4-yl-9H- | 5d | 209 | 876 | | |
| xanthene-3-carboxylic acid | | | | | |
| ethylamide | | | | | |
| 9-Piperidin-4-yl-6-pyridin-3-yl-9H- | 3i | 242 | >10000 | | |
| xanthen-4-ol | | | | | |
| 5-Chloro-9-piperidin-4-yl-9H- | 6k | 274 | 5701 | | |
| xanthene-3-carboxylic acid | | | | | |
| diethylamide | | | | | |
| 5-Methoxy-9-piperidin-4-yl-9H- | 6c | 277 | >10000 | | |
| xanthene-3-carboxylic acid | | | | | |
| diethylamide | | | | | |
| 3-[9-(1-Furan-3-ylmethyl-piperidin-4- | 5f | 303 | 3156 | | |
| yl)-9H-xanthen-3-yl]-pyridine | | | | | |
| 3-(5-Methoxy-9-piperidin-4-yl-9H- | 7b | 318 | >10000 | | |
| xanthen-3-yl)-pyridine | | | | | |
| (3-(S)-Hydroxy-pyrrolidin-1-yl)-(9- | 4n | 434 | >10000 | | |
| piperidin-4-yl-9H-xanthen-3-yl)- | | | | | |
| methanone | | | | | |
| 9-(1-Thiophen-2-ylmethyl-piperidin- | 15a | 450 | 4122 | | |
| 4-yl)-9H-xanthene-3-carboxylic acid | | | | | |
| diethylamide | | | | | |
| 1-Furan-3-ylmethyl-4-[3-(1H-tetrazol- | 4l | 498.1 | 6320 | | |
| 5-yl)-9H-xanthen-9-yl]-piperidine | | | | | |
| 4-(5-Methoxy-9-piperidin-4-yl-9H- | 8b | 619 | >10000 | | |
| xanthen-3-yl)-pyridine | | | | | |
| 3-(9-Piperidin-4-yl-9H-xanthen-3-yl)- | 2m | 634 | >10000 | | |
| 4H-[1,2,4]oxadiazol-5-one | | | | | |
| N-[2-(5-Methoxy-9-piperidin-4-yl-9H- | 9b | 760.95 | 4449 | | |
| xanthen-3-yl)-phenyl]-acetamide | | | | | |
| 4-[3-(1H-Tetrazol-5-yl)-9H-xanthen- | 3l | 768 | >10000 | | |
| 9-yl]-piperidine | | | | | |
| 3-[9-(1-Benzyl-piperidin-4-yl)-9H- | 7f | 783 | 3120 | | |
| xanthen-3-yl]-pyridine | | | | | |
| 4-[9-(1-Furan-3-ylmethyl-piperidin-4- | 3h | 791 | 4261 | | |
| yl)-9H-xanthen-3-yl]-pyridine | | | | | |
| N-[2-(9-Piperidin-4-yl-9H-xanthen-3- | 2g | 1020 | >10000 | | |
| yl)-phenyl]-acetamide | | | | | |
| 9-(1-Cyclopropylmethyl-piperidin-4- | 14a | 1085 | 6990 | | |
| yl)-9H-xanthene-3-carboxylic acid | | | | | |
| diethylamide | | | | | |
| N-{2-[9-(1-Allyl-piperidin-4-yl)-9H- | 6g | 1093 | 9629 | | |
| xanthen-3-yl]-phenyl}-acetamide | | | | | |
| 9-(1-Methyl-piperidin-4-yl)-9H- | 16a | 1143 | 35240 | | |
| xanthene-3-carboxylic acid | | | | | |
| diethylamide | | | | | |
| 4-[9-(1-Pyridin-2-ylmethyl-piperidin- | 4h | 2641 | 2053 | | |
| 4-yl)-9H-xanthen-3-yl]-pyridine | | | | | |
| 9-(1-Pyridin-2-ylmethyl-piperidin-4- | 11a | 2702 | 15090 | | |
| yl)-9H-xanthene-3-carboxylic acid | | | | | |
| diethylamide | | | | | |
| 3-[9-(1-Phenethyl-piperidin-4-yl)-9H- | 8f | 2992 | 907 | | |
| xanthen-3-yl]-pyridine | | | | | |
| 9-(1-Phenethyl-piperidin-4-yl)-9H- | 12a | 3831 | 976 | | |
| xanthene-3-carboxylic acid | | | | | |
| diethylamide | | | | | |
| 4-(9-Piperidin-4-yl-9H-xanthen-3-yl)- | 2h | 4533 | >10000 | | |
| pyridine | | | | | |
| N-{2-[9-(1-Phenethyl-piperidin-4-yl)- | 5g | 4571 | 995 | | |
| 9H-xanthen-3-yl]-phenyl}-acetamide | | | | | |
| (3-(S)-Hydroxy-pyrrolidin-1-yl)-[9-(1- | 6n | 7052.5 | 3404.5 | | |
| phenethyl-piperidin-4-yl)-9H- | | | | | |
| xanthen-3-yl]-methanone | | | | | |
| [9-(1-Allyl-piperidin-4-yl)-9H-xanthen- | 7n | 9225.0 | | | |
| 3-yl]-(3-(S)-hydroxy-pyrrolidin-1-yl)- | | | | | |
| methanone | | | | | |
| 6-Pyridin-3-yl-9-(1-thiophen-2- | 4i | 96.7 | | | |
| ylmethyl-piperidin-4-yl)-9H-xanthen- | | | | | |
| 4-ol | | | | | |
| 9-(1-Benzyl-piperidin-4-yl)-6-pyridin- | 5i | 94.0 | | | |
| 3-yl-9H-xanthen-4-ol | | | | | |
| N-[2-(5-Methoxy-9-piperidin-4-yl-9H- | 13q | 109.7 | | | |
| thioxanthen-3-yl)-phenyl]-acetamide | | | | | |
| N-{2-[5-Hydroxy-9-(1-pyridin-2- | 5e | 0.10 | | | |
| ylmethyl-piperidin-4-yl)-9H-xanthen- | | | | | |
| 3-yl]-phenyl}-acetamide | | | | | |
| N-{2-[5-Hydroxy-9-(1-thiophen-2- | 6e | 0.1 | | | |
| ylmethyl-piperidin-4-yl)-9H-xanthen- | | | | | |
| 3-yl]-phenyl}-acetamide | | | | | |
| 3-(5-Methoxy-9-piperidin-4-yl-9H- | 9q | 87.8 | | | |
| thioxanthen-3-yl)-pyridine | | | | | |
| 4-(5-Methoxy-9-piperidin-4-yl-9H- | 11q | 68.1 | | | |
| thioxanthen-3-yl)-pyridine | | | | | |
| 9-(1-Allyl-piperidin-4-yl)-6-pyridin-3- | 6i | 27.5 | | | |
| yl-9H-xanthen-4-ol | | | | | |
| N,N-Diethyl-9-(1-furan-3-ylmethyl- | 3p | 16.1 | | | |
| piperidin-4-yl)-9H-xanthene-3- | | | | | |
| carboxamidine | | | | | |
| 9-Piperidin-4-yl-6-pyridin-4-yl-9H- | 12q | 0.7 | | | |
| thioxanthen-4-ol | | | | | |
| 9-Piperidin-4-yl-6-pyridin-3-yl-9H- | 10q | 0.2 | | | |
| thioxanthen-4-ol | | | | | |
| N-[2-(5-Hydroxy-9-piperidin-4-yl-9H- | 14q | 0.2 | | | |
| thioxanthen-3-yl)-phenyl]-acetamide | | | | | |
| N-{2-[9-(1-Allyl-piperidin-4-yl)-5- | 16q | 121.4 | | | |
| hydroxy-9H-thioxanthen-3-yl]- | | | | | |
| phenyl}-acetamide | | | | | |
| 6-Pyridin-4-yl-9-(1-pyridin-2- | 15q | 0.1 | | | |
| ylmethyl-piperidin-4-yl)-9H- | | | | | |
| thioxanthen-4-ol | | | | | |
| 6-Pyridin-3-yl-9-(1-pyridin-2- | 18q | 0.4 | | | |
| ylmethyl-piperidin-4-yl)-9H- | | | | | |
| thioxanthen-4-ol | | | | | |
| 9-Piperidin-4-yl-6-(1H-tetrazol-5-yl)- | 4r | 14.0 | | | |
| 9H-xanthen-4-ol | | | | | |
| 5-Hydroxy-9-piperidin-4-yl-9H- | 5r | 165.60 | | | |
| xanthene-3-carbonitrile | | | | | |
| N-{2-[5-Hydroxy-9-(1-pyridin-2- | 19q | 0.1 | | | |
| ylmethyl-piperidin-4-yl)-9H- | | | | | |
| thioxanthen-3-yl]-phenyl}-acetamide | | | | | |

We claim:

1. A compound of Formula (I): 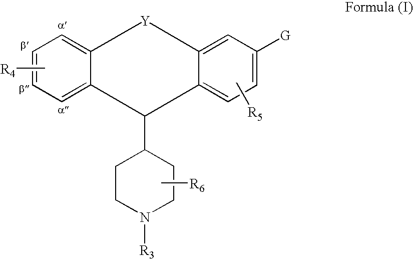
wherein:
G is —C(Z)N(R₁)R₂, C₆₋₁₀aryl, or a heterocycle selected from the group consisting of imidazolyl, triazolyl, tetrazolyl, oxadiazolyl, thiadiazolyl, oxathiadiazolyl, imidazolinyl, tetrahydropyrimidinyl, thienyl, pyrazolyl, pyrimidinyl, triazinyl, furyl, indazolyl, indolyl, indolinyl, isothiazolyl, isoxazolyl, oxazolyl, isoxadiazolyl, benzoxazolyl, quinolinyl, isoquinolinyl, and pyridinyl; wherein aryl and the heterocycles of G are optionally substituted with one to three substituents independently selected from the group consisting of C₁₋₈alkanyl, C₂₋₈alkenyl, C₂₋₈alkynyl, C₁₋₈alkanyloxy, hydroxy(C₁₋₈)alkanyl, carboxy(C₁₋₈)alkanyl, C₁₋₈alkanylcarbonylamino, halogen, hydroxy, cyano, nitro, oxo, thioxo, amino, C₁₋₆alkanylamino, di(C₁₋₆alkanyl)amino, C₁₋₈alkanylthio, C₁₋₈alkanylsulfonyl, C₁₋₈alkanylsulfonylamino, aminocarbonyl, aminothiocarbonyl, aminocarbonylamino, aminothiocarbonylamino, C₁₋₈alkanylaminocarbonyl, di(C₁₋₈alkanyl)aminocarbonyl, and C₁₋₆alkanyloxycarbonylamino;
R₁ is a substituent selected from the group consisting of hydrogen, C₁₋₈alkanyl, C₂₋₈alkenyl, and C₂₋₈alkynyl;
R₂ is a substituent selected from the group consisting of hydrogen; C₁₋₈alkanyl; C₂₋₈alkenyl; C₂₋₈alkynyl; C₆₋₁₀aryl; and
C₁₋₈cycloalkanyl;
wherein C₁₋₈alkanyl is optionally substituted with one to three substituents independently selected from the group consisting of phenyl, amino, C₁₋₆alkanylamino, di(C₁₋₆alkanyl)amino, C₁₋₆alkanyloxy, thioC₁₋₆alkanyloxy, hydroxy, fluoro, chloro, cyano, aminocarbonyl, C₁₋₈alkanylaminocarbonyl, di(C₁₋₈alkanyl)aminocarbonyl, C₁₋₆alkanyloxycarbonyl, and aryloxy; and wherein any aryl-containing substituents and C₁₋₈cycloalkanyl substituents of R₂ are optionally substituted with one to three substituents independently selected from the group consisting of C₁₋₈alkanyl, C₂₋₈alkenyl, C₂₋₈alkynyl, C₁₋₈alkanyloxy, trifluoromethyl, trifluoromethoxy, phenyl, halogen, cyano, hydroxy, C₁₋₈alkanylthio, C₁₋₈alkanylsulfonyl, and C₁₋₈alkanylsulfonylamino; or R₁ and R₂ taken together with the nitrogen to which they are attached form a 5-7 membered cycloheteroalkyl optionally substituted with one to three substituents independently selected from the group consisting of C₁₋₈alkanyl, hydroxy(C₁₋₈)alkanyl, hydroxy, amino, C₁₋₆alkanylamino, di(C₁₋₆alkanyl)amino, and halogen;
R₃ is a substituent selected from the group consisting of hydrogen, C₁₋₈alkanyl, halo₁₋₃(C₁₋₈)alkanyl, C₂₋₈alkenyl, C₂₋₈alkynyl, C₃₋₈cycloalkanyl, cycloalkanyl(C₁₋₈)alkanyl, C₁₋₈alkanyloxy(C₁₋₈)alkanyl, C₁₋₈alkanylthio(C₁₋₈)alkanyl, hydroxyC₁₋₈alkanyl, C₁₋₈alkanyloxycarbonyl, halo₁₋₃(C₁₋₈)alkanylcarbonyl, formyl, thioformyl, carbamimidoyl, phenylimino(C₁₋₈)alkanyl, phenyl(C₁₋₈)alkanyl, phenyl(C₁₋₈)alkenyl, phenyl(C₁₋₈)alkynyl, naphthyl(C₁₋₈)alkanyl and heteroaryl(C₁₋₈)alkanyl wherein the heteroaryl is selected from the group consisting of benzo[1,3]dioxolyl, imidazolyl, furanyl, pyridinyl, thienyl, indazolyl, indolyl, indolinyl, isoindolinyl, isoquinolinyl, isothiazolyl, isoxazolyl, oxazolyl, pyrazinyl, pyrazolyl, pyridazinyl, pyrimidinyl, pyrrolyl, quinolinyl, isoquinolinyl, tetrazolyl, thiazolyl; wherein phenyl, naphthyl, and heteroaryl are optionally substituted with one to three substituents independently selected from the group consisting of C₁₋₆alkanyl, C₂₋₆alkenyl, C₁₋₆alkanyloxy, amino, C₁₋₆alkanylamino, di(C₁₋₆alkanyl)amino, C₁₋₆alkanylcarbonyl, C₁₋₆alkanylcarbonyloxy, C₁₋₆alkanylcarbonylamino, C₁₋₆alkanylthio, C₁₋₆alkanylsulfonyl, halogen, hydroxy, cyano, fluoro(C₁₋₆)alkanyl, thioureido, and fluoro(C₁₋₆)alkanyloxy; alternatively, when phenyl and heteroaryl are optionally substituted with alkanyl or alkanyloxy substituents attached to adjacent carbon atoms, the two substituents can together form a fused cyclic alkanyl or cycloheteroalkanyl selected from the group consisting of —(CH₂)₃₋₅—, —O(CH₂)₂₋₄—(CH₂)₂₋₄O—, and —O(CH₂)₁₋₃O—;
R₄ is one to three substituents independently selected from the group consisting of hydrogen; C₁₋₆alkanyl; C₂₋₆alkenyl; C₂₋₆alkynyl; aryl(C₂₋₆)alkynyl; C₁₋₆alkanyloxy; amino; C₁₋₆alkanylamino; di(C₁₋₆alkanyl)amino; C₆₋₁₀arylamino wherein C₆₋₁₀aryl is optionally substituted with one to three substitutents independently selected from the group consisting of C₁₋₆alkanyl, C₁₋₆alkoxy, halogen, and hydroxy; formylamino; pyridinylamino; C₁₋₆alkanylcarbonyl; C₁₋₆alkanylcarbonyloxy; C₁₋₆alkanyloxycarbonyl; aminocarbonyl; C₁₋₆alkanylaminocarbonyl; di(C₁₋₆alkanyl)aminocarbonyl; C₁₋₆alkanylcarbonylamino; C₁₋₆alkanylthio; C₁₋₆alkanylsulfonyl; halogen; hydroxy; cyano; hydroxycarbonyl; C₆₋₁₀aryl; chromanyl; chromenyl; furanyl; imidazolyl; indazolyl; indolyl; indolinyl; isoindolinyl; isoquinolinyl; isothiazolyl; isoxazolyl; naphthyridinyl; oxazolyl; pyrazinyl; pyrazolyl; pyridazinyl; pyridinyl; pyrimidinyl; pyrrolyl; quinazolinyl; quinolinyl; quinolizinyl; quinoxalinyl; tetrazolyl; thiazolyl; thienyl; fluoroalkanyl and fluoroalkanyloxy; or optionally, when R₄ is two substituents attached to adjacent carbon atoms, the two substituents together form a single fused moiety; wherein the fused moiety is —(CH₂)₃₋₅—, —O(CH₂)₂₋₄—(CH₂)₂₋₄O—, —O(CH₂)₁₋₃O—, or —S—C(NH₂)═N—;
R₅ is one to two substituents independently selected from the group consisting of hydrogen, C₁₋₆alkanyl, C₂₋₆alkenyl, C₁₋₆alkanyloxy, amino, C₁₋₆alkanylamino, di(C₁₋₆alkanyl)amino, C₁₋₆alkanylcarbonyl, C₁₋₆alkanylcarbonyloxy, C₁₋₆alkanyloxycarbonyl, C₁₋₆alkanylaminocarbonyl, C₁₋₆alkanylcarbonylamino, C₁₋₆alkanylthio, C₁₋₆alkanylsulfonyl, halogen, hydroxy, cyano, fluoro(C₁₋₆)alkanyl and fluoro(C₁₋₆)alkanyloxy;
R₆ is one to four substituents independently selected from the group consisting of hydrogen, C₁₋₆alkanyl, C₂₋₆alkenyl, C₁₋₆alkanyloxy, amino, C₁₋₆alkanylamino, di(C₁₋₆alkanyl)amino, C₁₋₆alkanylcarbonyl, C₁₋₆alkanylcarbonyloxy, C₁₋₆alkanyloxycarbonyl, C₁₋₆alkanylaminocarbonyl, C₁₋₆alkanylcarbonylamino, C₁₋₆alkanylthio, C₁₋₆alkanylsulfonyl, halogen, hydroxy, cyano, fluoro(C₁₋₆)alkanyl and fluoro(C₁₋₆)alkanyloxy;
Y is O or S;
Z is O, S, NH, N(C₁₋₆alkanyl), N(OH), N(OC₁₋₆alkanyl), or N(phenyl); and enantiomers, diastereomers, tautomers, solvates, or pharmaceutically acceptable salts thereof.

2. The compound according to claim 1 wherein G is —C(Z)N(R₁)R₂, phenyl, or a heterocycle selected from the group consisting of imidazolyl, triazolyl, tetrazolyl, oxadiazolyl, thiadiazolyl, oxathiadiazolyl, imidazolinyl, tetrahydropyrimidinyl, thienyl, pyrazolyl, pyrimidinyl, triazinyl, isothiazolyl, isoxazolyl, oxazolyl, isoxadiazolyl, and pyridinyl; wherein phenyl and the heterocycles of G are optionally substituted with one to three substituents independently selected from the group consisting of C₁₋₈alkanyl, C₁₋₈alkanyloxy, hydroxy(C₁₋₈)alkanyl, carboxy(C₁₋₈)alkanyl, C₁₋₈alkanylcarbonylamino, halogen, hydroxy, cyano, oxo, thioxo, amino, C₁₋₆alkanylamino, di(C₁₋₆alkanyl)amino, C₁₋₈alkanylthio, aminocarbonyl, aminothiocarbonyl, C₁₋₈alkanylaminocarbonyl, di(C₁₋₈alkanyl)aminocarbonyl, and C₁₋₆alkanyloxycarbonylamino.

3. The compound according to claim 1 wherein G is —C(Z)N(R₁)R₂, phenyl, or a heterocycle selected from the group consisting of imidazolyl, tetrazolyl, oxadiazolyl, thiadiazolyl, oxathiadiazolyl, imidazolinyl, thienyl, pyrazolyl, pyrimidinyl, triazinyl, isothiazolyl, isoxazolyl, oxazolyl, isoxadiazolyl, and pyridinyl; wherein phenyl and the heterocycles of G are optionally substituted with one to three substituents independently selected from the group consisting of C₁₋₄alkanyl, C₁₋₄alkanyloxy, hydroxy(C₁₋₄)alkanyl, carboxy(C₁₋₄)alkanyl, C₁₋₄alkanylcarbonylamino, hydroxy, cyano, oxo, thioxo, amino, C₁₋₆alkanylamino, di(C₁₋₆alkanyl)amino, C₁₋₈alkanylthio, aminocarbonyl, aminothiocarbonyl, C₁₋₈alkanylaminocarbonyl, and di(C₁₋₈alkanyl)aminocarbonyl.

4. The compound according to claim 1 wherein G is —C(Z)N(R₁)R₂, phenyl, or a heterocycle selected from the group consisting of imidazolyl, tetrazolyl, oxadiazolyl, thiadiazolyl, oxathiadiazolyl, thienyl, isothiazolyl, isoxazolyl, isoxadiazolyl, and pyridinyl; wherein phenyl and the heterocycles of G are optionally substituted with one to three substituents independently selected from the group consisting of C₁₋₄alkanyl, C₁₋₄alkanyloxy, hydroxy(C₁₋₄)alkanyl, C₁₋₄alkanylcarbonylamino, hydroxy, cyano, oxo, thioxo, and aminocarbonyl.

5. The compound according to claim 1 wherein G is —C(Z)N(R₁)R₂, phenyl, or a heterocycle selected from the group consisting of tetrazolyl, oxadiazolyl, and pyridinyl; wherein phenyl and the heterocycles of G are optionally substituted with one to three substituents independently selected from the group consisting of C₁₋₄alkanylcarbonylamino and oxo.

6. The compound according to claim 1 wherein G is N,N-diethylaminocarbonyl, 3-(N,N-diethylaminocarbonyl)-phenyl, 2-methylcarbonylaminophenyl, N—N-diethylamidino, pyridin-3-yl, 3-(S)hydroxypyrrolidin-1-ylcarbonyl, N-ethylaminocarbonyl, 1H-tetrazol-4-yl, pyridine-4-yl, or 4H-[1,2,4]-oxadiazol-5-oxo-3-yl.

7. The compound according to claim 1 wherein R₁ is a substituent selected from the group consisting of hydrogen and C₁₋₄alkanyl.

8. The compound according to claim 1 wherein R₁ is selected from the group consisting of hydrogen, methyl, ethyl, and propyl.

9. The compound according to claim 1 wherein R₁ is selected from the group consisting of hydrogen, methyl, and ethyl.

10. The compound according to claim 1 wherein R₁ is selected from the group consisting of hydrogen and ethyl.

11. The compound according to claim 1 wherein R₂ is selected from the group consisting of hydrogen; C₁₋₄alkanyl; phenyl; and C₁₋₆cycloalkanyl; wherein C₁₋₄alkanyl is optionally substituted with one to three substituents independently selected from the group consisting of phenyl, amino, C₁₋₆alkanylamino, di(C₁₋₆alkanyl)amino, C₁₋₄alkanyloxy, hydroxy, fluoro, chloro, cyano, aminocarbonyl, C₁₋₈alkanylaminocarbonyl, di(C₁₋₈alkanyl)aminocarbonyl, and phenoxy; and wherein any phenyl-containing substituents and C₁₋₆cycloalkanyl substituents of R₂ are optionally substituted with one to three substituents independently selected from the group consisting of C₁₋₈alkanyl, C₁₋₈alkanyloxy, trifluoromethyl, phenyl, fluoro, hydroxy, C₁₋₈alkanylthio, C₁₋₈alkanylsulfonyl, and C₁₋₈alkanylsulfonylamino; or R₁ and R₂ taken together with the nitrogen to which they are attached form a 5-7 membered cycloheteroalkyl optionally substituted with one to three substituents independently selected from the group consisting of C₁₋₄alkanyl, hydroxy(C₁₋₄)alkanyl, hydroxy, amino, C₁₋₆alkanylamino, di(C₁₋₆alkanyl)amino, and fluoro.

12. The compound according to claim 1 wherein R₂ is selected from the group consisting of hydrogen, C₁₋₄alkanyl, phenyl, and C₁₋₆cycloalkanyl; wherein C₁₋₄alkanyl is optionally substituted with one to three substituents independently selected from the group consisting of phenyl, C₁₋₄alkanyloxy, hydroxy, fluoro, aminocarbonyl, C₁₋₈alkanylaminocarbonyl, di(C₁₋₈alkanyl)aminocarbonyl, and phenoxy; and wherein any phenyl-containing substituent of R₂ is optionally substituted with one to three substituents independently selected from the group consisting of C₁₋₆alkanyl, C₁₋₆alkanyloxy, fluoro, hydroxy, and C₁₋₆alkanylthio; or R₁ and R₂ taken together with the nitrogen to which they are attached form a pyrrolidinyl or piperidinyl ring wherein said pyrrolidinyl or piperidinyl is optionally substituted with a substituent selected from the group consisting of C₁₋₄alkanyl and hydroxy.

13. The compound according to claim 1 wherein R₂ is selected from the group consisting of hydrogen, C₁₋₄alkanyl and phenyl; wherein C₁₋₄alkanyl is optionally substituted with one to three substituents independently selected from the group consisting of phenyl, C₁₋₄alkanyloxy, hydroxy, fluoro, and phenoxy; and wherein any phenyl-containing substituent of R₂ is optionally substituted with one to three substituents independently selected from the group consisting of C₁₋₆alkanyl, C₁₋₆alkanyloxy, fluoro, and hydroxy; or R₁ and R₂ taken together with the nitrogen to which they are attached form a pyrrolidinyl or piperidinyl ring wherein said pyrrolidinyl or piperidinyl is optionally substituted with a substituent selected from the group consisting of C₁₋₃alkanyl and hydroxy.

14. The compound according to claim 1 wherein R₂ is selected from the group consisting of hydrogen and C₁₋₄alkanyl, or R₁ and R₂ taken together with the nitrogen to which they are attached form a pyrrolidinyl ring optionally substituted with hydroxy.

15. The compound according to claim 1 wherein R₂ is hydrogen or ethyl, or R₁ and R₂ taken together with the nitrogen to which they are attached form a pyrrolidinyl ring optionally substituted with hydroxy.

16. The compound according to claim 1 wherein R₃ is selected from the group consisting of hydrogen, C₁₋₈alkanyl, C₂₋₈alkenyl, C₂₋₈alkynyl, C₁₋₈alkanyloxy(C₁₋₈)alkanyl, C₁₋₈alkanylthio(C₁₋₈)alkanyl, hydroxyC₁₋₈alkanyl, thioformyl, phenylimino(C₁₋₈)alkanyl, phenyl(C₁₋₈)alkanyl, and heteroaryl(C₁₋₈)alkanyl wherein heteroaryl is selected from the group consisting of benzo[1,3]dioxolyl, imidazolyl, furanyl, pyridinyl, thienyl, indolyl, indolinyl, isoquinolinyl, pyrazinyl, pyrazolyl, pyridazinyl, pyrimidinyl, pyrrolyl, quinolinyl, isoquinolinyl, tetrazolyl; wherein phenyl and heteroaryl are optionally substituted with one to three substituents independently selected from the group consisting of C₁₋₆alkanyloxy and hydroxy; or optionally, when phenyl and heteroaryl are optionally substituted with two substituents attached to adjacent carbon atoms, the two substituents together form a single fused moiety; wherein the moiety is selected from —O(CH₂)₁₋₃O—.

17. The compound according to claim 1 wherein R₃ is selected from the group consisting of hydrogen, methyl, allyl, 2-methyl-allyl, propynyl, hydroxyethyl, methylthioethyl, methoxyethyl, thioformyl, phenyliminomethyl, phenethyl, and heteroaryl(C₁₋₈)alkanyl wherein the heteroaryl is selected from the group consisting of benzo[1,3]dioxolyl, imidazolyl, furanyl, pyridinyl, thienyl, pyrimidinyl, pyrrolyl, quinolinyl, isoquinolinyl, tetrazolyl; wherein the phenyl in any phenyl-containing substituent is optionally substituted with one hydroxyl group.

18. The compound according to claim 1 wherein R₃ is hydrogen, C₁₋₈alkanyl, C₂₋₈alkenyl, C₃₋₈cycloalkanyl, phenyl(C₁₋₈)alkanyl, or heteroaryl(C₁₋₈)alkanyl wherein the heteroaryl is imidazolyl, furanyl, pyridinyl, or thienyl.

19. The compound according to claim 1 wherein R₃ is hydrogen, methyl, allyl, or heteroarylmethyl wherein heteroaryl is selected from the group consisting of benzo[1,3]dioxolyl, imidazolyl, furanyl, pyridinyl, and thienyl.

20. The compound according to claim 1 wherein R₃ is hydrogen, methyl, ethyl, propenyl, cyclopropylmethyl, benzyl, phenethyl, or heteroarylmethyl wherein the heteroaryl is furanyl, imidazolyl, pyridinyl, or thienyl.

21. The compound according to claim 1 wherein R₄ is one to three substituents independently selected from the group consisting of hydrogen; C₁₋₆alkanyl; C₁₋₆alkanyloxy; C₆₋₁₀arylamino wherein C₆₋₁₀aryl is optionally substituted with one to three substitutents independently selected from the group consisting of C₁₋₆alkanyl, C₁₋₆alkoxy, halogen, and hydroxy; formylamino; pyridinylamino; aminocarbonyl; C₁₋₆alkanylaminocarbonyl; C₁₋₆alkanylcarbonylamino; halogen; hydroxy; C₆₋₁₀aryl; chromanyl; chromenyl; furanyl; imidazolyl; indazolyl; indolyl; indolinyl; isoindolinyl; isoquinolinyl; isothiazolyl; isoxazolyl; naphthyridinyl; oxazolyl; pyrazinyl; pyrazolyl; pyridazinyl; pyridinyl; pyrimidinyl; pyrrolyl; quinazolinyl; quinolinyl; quinolizinyl; quinoxalinyl; tetrazolyl; thiazolyl; and thienyl.

22. The compound according to claim 1 wherein R₄ is one to two substituents independently selected from the group consisting of hydrogen, C₁₋₄alkanyl, C₁₋₄alkanyloxy, halogen, phenyl, furanyl, imidazolyl, indazolyl, indolyl, indolinyl, isoindolinyl, isoquinolinyl, isothiazolyl, isoxazolyl, oxazolyl, pyrazinyl, pyrazolyl, pyridazinyl, pyridinyl, pyrimidinyl, pyrrolyl, quinolinyl, tetrazolyl, thiazolyl, thienyl, and hydroxy.

23. The compound according to claim 1 wherein R₄ is one to two substituents independently selected from the group consisting of hydrogen, methyl, methoxy, bromo, fluoro, α′- or β′-phenyl, α′- or β′-pyridinyl, α′- or β′-furanyl, and hydroxy.

24. The compound according to claim 1 wherein R₄ is one substituent selected from the group consisting of hydrogen, methoxy, chloro, and hydroxy.

25. The compound according to claim 1 wherein R₄ is one substituent and is hydrogen or hydroxy.

26. The compound according to claim 1 wherein R₅ is one to two substituents independently selected from the group consisting of hydrogen and halogen.

27. The compound according to claim 1 wherein R₅ is hydrogen.

28. The compound according to claim 1 wherein R₆ is one to four substituents independently selected from the group consisting of hydrogen, C₁₋₆alkanyl, C₂₋₆alkenyl, C₁₋₆alkanyloxy, halogen, hydroxy, fluoro(C₁₋₆)alkanyl and fluoro(C₁₋₆)alkanyloxy.

29. The compound according to claim 1 wherein R₆ is one to two substituents independently selected from the group consisting of hydrogen and C₁₋₄alkanyl.

30. The compound according to claim 1 wherein R₆ is one to two substituents independently selected from the group consisting of hydrogen and methyl.

31. The compound according to claim 1 wherein R₆ is hydrogen.

32. The compound according to claim 1 wherein Y is O or S.

33. The compound according to claim 1 wherein Y is O.

34. The compound according to claim 1 wherein Z is O, NH, N(C₁₋₆alkanyl), N(OH), N(OC₁₋₆alkanyl), or N(phenyl).

35. The compound according to claim 1 wherein Z is O, NH, or N(OH).

36. The compound according to claim 1 wherein Z is O or NH.

37. The compound according to claim 1 wherein Z is O.

38. The compound according to claim 1 wherein R₄ is hydrogen and Y is O.

39. The compound according to claim 1 wherein R₄ is α′-hydroxy and Y is O.

40. The compound according to claim 1 wherein R₄ is hydrogen and Y is S.

41. The compound according to claim 1 wherein R₄ is α′-hydroxy and Y is S.

42. A compound of Formula (I): 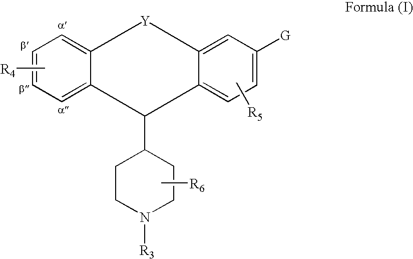
wherein:
G is independently selected from —C(Z)N(R₁)R₂, phenyl, or a heterocycle selected from the group consisting of imidazolyl, triazolyl, tetrazolyl, oxadiazolyl, thiadiazolyl, oxathiadiazolyl, imidazolinyl, tetrahydropyrimidinyl, thienyl, pyrazolyl, pyrimidinyl, triazinyl, isothiazolyl, isoxazolyl, oxazolyl, isoxadiazolyl, and pyridinyl; wherein phenyl and the heterocycles of G are optionally substituted with one to three substituents independently selected from the group consisting of C₁₋₈alkanyl, C₁₋₈alkanyloxy, hydroxy(C₁₋₈)alkanyl, carboxy(C₁₋₈)alkanyl, C₁₋₈alkanylcarbonylamino, halogen, hydroxy, cyano, oxo, thioxo, amino, C₁₋₆alkanylamino, di(C₁₋₆alkanyl)amino, C₁₋₈alkanylthio, aminocarbonyl, aminothiocarbonyl, C₁₋₈alkanylaminocarbonyl, di(C₁₋₈alkanyl)aminocarbonyl, and C₁₋₆alkanyloxycarbonylamino; provided that when G is pyridin-3-yl or thien-3-yl and R³ is hydrogen, R⁴ is other than hydrogen;
R₁ is hydrogen or C₁₋₄alkanyl;
R₂ is selected from the group consisting of hydrogen; C₁₋₄alkanyl; phenyl; and C₁₋₆cycloalkanyl; wherein C₁₋₄alkanyl is optionally substituted with one to three substituents independently selected from the group consisting of phenyl, amino, C₁₋₆alkanylamino, di(C₁₋₆alkanyl)amino, C₁₋₄alkanyloxy, hydroxy, fluoro, chloro, cyano, aminocarbonyl, C₁₋₈alkanylaminocarbonyl, di(C₁₋₈alkanyl)aminocarbonyl, and phenoxy; and wherein any phenyl-containing substituent of R₂ and C₁₋₆cycloalkanyl substituents of R₂ are optionally substituted with one to three substituents independently selected from the group consisting of C₁₋₈alkanyl, C₁₋₈alkanyloxy, trifluoromethyl, phenyl, fluoro, hydroxy, C₁₋₈alkanylthio, C₁₋₈alkanylsulfonyl, and C₁₋₈alkanylsulfonylamino; or R₁ and R₂ taken together with the nitrogen to which they are attached form a 5-7 membered heterocycloalkyl wherein said heterocycloalkyl is optionally substituted with one to three substituents independently selected from the group consisting of C₁₋₈alkanyl, hydroxy(C₁₋₈)alkanyl, and hydroxy;
R₃ is selected from the group consisting of hydrogen, C₁₋₈alkanyl, C₂₋₈alkenyl, C₂₋₈alkynyl, C₁₋₈alkanyloxy(C₁₋₁₈)alkanyl, C₁₋₈alkanylthio(C₁₋₈)alkanyl, hydroxyC₁₋₈alkanyl, thioformyl, phenylimino(C₁₋₈)alkanyl, phenyl(C₁₋₈)alkanyl, and heteroaryl(C₁₋₈)alkanyl wherein heteroaryl is selected from the group consisting of benzo[1,3]dioxolyl, imidazolyl, furanyl, pyridinyl, thienyl, indolyl, indolinyl, isoquinolinyl, pyrazinyl, pyrazolyl, pyridazinyl, pyrimidinyl, pyrrolyl, quinolinyl, isoquinolinyl, tetrazolyl; wherein phenyl and heteroaryl are optionally substituted with one to three substituents independently selected from the group consisting of C₁₋₆alkanyloxy and hydroxy; or optionally, when phenyl and heteroaryl are optionally substituted with two substituents attached to adjacent carbon atoms, the two substituents together form a single fused moiety; wherein the moiety is selected from —O(CH₂)₁₋₃O—;
R₄ is one to three substituents independently selected from the group consisting of hydrogen; C₁₋₆alkanyl; C₁₋₆alkanyloxy; C₆₋₁₀arylamino wherein C₆₋₁₀aryl is optionally substituted with one to three substitutents independently selected from the group consisting of C₁₋₆alkanyl, C₁₋₆alkoxy, halogen, and hydroxy; formylamino; pyridinylamino; aminocarbonyl; C₁₋₆alkanylaminocarbonyl; C₁₋₆alkanylcarbonylamino; halogen; hydroxy; C₆₋₁₀aryl; chromanyl; chromenyl; furanyl; imidazolyl; indazolyl; indolyl; indolinyl; isoindolinyl; isoquinolinyl; isothiazolyl; isoxazolyl; naphthyridinyl; oxazolyl; pyrazinyl; pyrazolyl; pyridazinyl; pyridinyl; pyrimidinyl; pyrrolyl; quinazolinyl; quinolinyl; quinolizinyl; quinoxalinyl; tetrazolyl; thiazolyl; and thienyl;
R₅ is one to two substituents independently selected from the group consisting of hydrogen and halogen;
R₆ is one to four substituents independently selected from the group consisting of hydrogen, C₁₋₆alkanyl, C₂₋₆alkenyl, C₁₋₆alkanyloxy, halogen, hydroxy, fluoro(C₁₋₆)alkanyl and fluoro(C₁₋₆)alkanyloxy.
Y is O or S;
Z is O, NH, N(C₁₋₆alkanyl), N(OH), N(OC₁₋₆alkanyl), or N(phenyl); and
enantiomers, diastereomers, tautomers, solvates, and pharmaceutically acceptable salts thereof.

43. A compound of Formula (J): 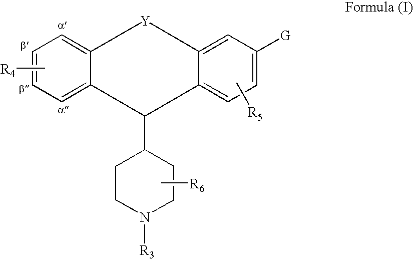
wherein:
G is selected from —C(Z)N(R₁)R₂, phenyl, or a heterocycle selected from the group consisting of imidazolyl, tetrazolyl, oxadiazolyl, thiadiazolyl, oxathiadiazolyl, imidazolinyl, thienyl, pyrazolyl, pyrimidinyl, triazinyl, isothiazolyl, isoxazolyl, oxazolyl, isoxadiazolyl, and pyridinyl; wherein phenyl and the heterocycles of G are optionally substituted with one to three substituents independently selected from the group consisting of C₁₋₄alkanyl, C₁₋₄alkanyloxy, hydroxy(C₁₋₄)alkanyl, carboxy(C₁₋₄)alkanyl, C₁₋₄alkanylcarbonylamino, hydroxy, cyano, oxo, thioxo, amino, C₁₋₆alkanylamino, di(C₁₋₆alkanyl)amino, C₁₋₈alkanylthio, aminocarbonyl, aminothiocarbonyl, C₁₋₈alkanylaminocarbonyl, and di(C₁₋₈alkanyl)aminocarbonyl;
R₁ is selected from the group consisting of hydrogen, methyl, ethyl, and propyl;
R₂ is selected from the group consisting of hydrogen, C₁₋₄alkanyl, phenyl, and C₁₋₆cycloalkanyl; wherein C₁₋₄alkanyl is optionally substituted with one to three substituents independently selected from the group consisting of phenyl, C₁₋₄alkanyloxy, hydroxy, fluoro, aminocarbonyl, C₁₋₈alkanylaminocarbonyl, di(C₁₋₈alkanyl)aminocarbonyl, and phenoxy; and wherein any phenyl-containing substituent of R₂ is optionally substituted with one to three substituents independently selected from the group consisting of C₁₋₆alkanyl, C₁₋₆alkanyloxy, fluoro, hydroxy, and C₁₋₆alkanylthio; or R₁ and R₂ taken together with the nitrogen to which they are attached form pyrrolidinyl or piperidinyl ring wherein said pyrrolidinyl or piperidinyl is optionally substituted with a substituent selected from the group consisting of C₁₋₃alkanyl and hydroxy;
R₃ is selected from the group consisting of hydrogen, methyl, allyl, 2-methyl-allyl, propynyl, hydroxyethyl, methylthioethyl, methoxyethyl, thioformyl, phenyliminomethyl, phenethyl, and heteroaryl(C₁₋₈)alkanyl wherein the heteroaryl is selected from the group consisting of benzo[1,3]dioxolyl, imidazolyl, furanyl, pyridinyl, thienyl, pyrimidinyl, pyrrolyl, quinolinyl, isoquinolinyl, tetrazolyl; wherein the phenyl in any phenyl-containing substituent is optionally substituted with one hydroxyl group;
R₄ is one to two substituents independently selected from the group consisting of hydrogen, C₁₋₄alkanyl, C₁₋₄alkanyloxy, halogen, phenyl, furanyl, imidazolyl, indazolyl, indolyl, indolinyl, isoindolinyl, isoquinolinyl, isothiazolyl, isoxazolyl, oxazolyl, pyrazinyl, pyrazolyl, pyridazinyl, pyridinyl, pyrimidinyl, pyrrolyl, quinolinyl, tetrazolyl, thiazolyl, thienyl, and hydroxy;
R₅ is hydrogen;
R₆ is one to two substituents independently selected from the group consisting of hydrogen and C₁₋₄alkanyl;
Y is O or S;
Z is O, NH, or N(OH); and
enantiomers, diastereomers, tautomers, solvates, and pharmaceutically acceptable salts thereof.

44. The compound according to claim 26 wherein G is —C(Z)N(R₁)R₂, phenyl, or a heterocycle selected from the group consisting of imidazolyl, tetrazolyl, oxadiazolyl, thiadiazolyl, oxathiadiazolyl, thienyl, isothiazolyl, isoxazolyl, isoxadiazolyl, and pyridinyl; wherein phenyl and the heterocycles of G are optionally substituted with one to three substituents independently selected from the group consisting of C₁₋₄alkanyl, C₁₋₄alkanyloxy, hydroxy(C₁₋₄)alkanyl, C₁₋₄alkanylcarbonylamino, hydroxy, cyano, oxo, thioxo, and aminocarbonyl.

45. The compound according to claim 26 wherein G is —C(Z)N(R₁)R₂, 2-methylcarbonylaminophenyl, 2-aminocarbonyl-phenyl, 1H-tetrazol-4-yl, 2-methyl-tetrazol-5-yl, 4H-[1,2,4]-oxadiazol-5-oxo-3-yl, 4H-[1,2,4]-oxadiazol-5-thioxo-3-yl, 4H-[1,2,4]thiadiazol-5-oxo-3-yl, [1,2,3,5]oxathiadiazol-2-oxo-4-yl, or pyridin-3-yl.

46. The compound according to claim 26 wherein R₂ is selected from the group consisting of hydrogen, C₁₋₄alkanyl and phenyl; wherein C₁₋₄alkanyl is optionally substituted with one to three substituents independently selected from the group consisting of phenyl, C₁₋₄alkanyloxy, hydroxy, fluoro, and phenoxy; and wherein any phenyl-containing substituent is optionally substituted with one to three substituents independently selected from the group consisting of C₁₋₆alkanyl, C₁₋₆alkanyloxy, fluoro, and hydroxy; or R₁ and R₂ taken together with the nitrogen to which they are attached form a pyrrolidinyl or piperidinyl ring wherein said pyrrolidinyl or piperidinyl is optionally substituted with a substituent selected from C₁₋₃alkanyl or hydroxy; and R₃ is a substituent selected from the group consisting of benzo[1,3]dioxol-5-ylmethyl, carbamimidoyl, 1-H-imidazol-4-ylmethyl, phenyliminomethyl, 1-prop-2-ynyl, thioformyl, 2-hydroxyphenyl-methyl, hydroxy-ethyl, methoxy-ethyl, 2-methyl-allyl, 2-methyl-but-2-enyl, allyl, furan-3-ylmethyl, H, Me, methylthioethyl, phenethyl, pyridin-2-yl methyl, and thiophen-2-yl methyl.

47. A compound of Formula (J): 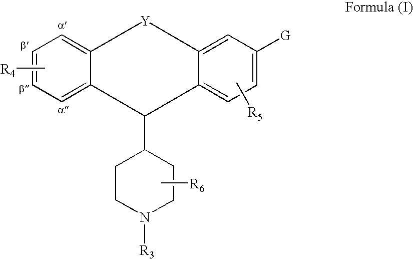
wherein:
G is selected from —C(Z)N(R₁)R₂, 2-methylcarbonylaminophenyl, 2-aminocarbonyl-phenyl, 1H-tetrazol-4-yl, 2-methyl-tetrazol-5-yl, 4H-[1,2,4]-oxadiazol-5-oxo-3-yl, 4H-[1,2,4]-oxadiazol-5-thioxo-3-yl, 4H-[1,2,4]thiadiazol-5-oxo-3-yl, [1,2,3,5]oxathiadiazol-2-oxo-4-yl, or pyridin-3-yl;
R₁ is hydrogen, methyl, or ethyl;
R₂ is selected from the group consisting of hydrogen, C₁₋₄alkanyl and phenyl; wherein C₁₋₄alkanyl is optionally substituted with one to three substituents independently selected from the group consisting of phenyl, C₁₋₄alkanyloxy, hydroxy, fluoro, and phenoxy; and wherein any phenyl-containing substituent of R₂ is optionally substituted with one to three substituents independently selected from the group consisting of C₁₋₆alkanyl, C₁₋₆alkanyloxy, fluoro, and hydroxy; or R₁ and R₂ taken together with the nitrogen to which they are attached form a pyrrolidinyl or piperidinyl ring wherein said pyrrolidinyl or piperidinyl is optionally substituted with a substituent selected from C₁₋₃alkanyl or hydroxy;
R₃ is selected from the group consisting of hydrogen, methyl, allyl, 2-methyl-allyl, propynyl, hydroxyethyl, methylthioethyl, methoxyethyl, thioformyl, phenyliminomethyl, phenethyl, and heteroaryl(C₁₋₈)alkanyl wherein the heteroaryl is selected from the group consisting of benzo[1,3]dioxolyl, imidazolyl, furanyl, pyridinyl, thienyl, pyrimidinyl, pyrrolyl, quinolinyl, isoquinolinyl, tetrazolyl; wherein the phenyl in any phenyl-containing substituent is optionally substituted with one hydroxyl group;
R₄ is one to three substituents independently selected from the group consisting of hydrogen, C₁₋₄alkanyl, C₁₋₄alkanyloxy, halogen, phenyl, furanyl, imidazolyl, indazolyl, indolyl, indolinyl, isoindolinyl, isoquinolinyl, isothiazolyl, isoxazolyl, oxazolyl, pyrazinyl, pyrazolyl, pyridazinyl, pyridinyl, pyrimidinyl, pyrrolyl, quinolinyl, tetrazolyl, thiazolyl, thienyl, and hydroxy;
R₅ is hydrogen;
R₆ is one to two substituents independently selected from the group consisting of hydrogen and methyl;
Y is O or S;
Z is O or NH; and
enantiomers, diastereomers, tautomers, solvates, and pharmaceutically acceptable salts thereof.

48. The compound according to claim 31 wherein R₂ is a substituent selected from the group consisting of hydrogen, C₁₋₄alkanyl and phenyl; wherein C₁₋₄alkanyl is optionally substituted with one to three substituents independently selected from the group consisting of phenyl, C₁₋₄alkanyloxy, hydroxy, and 2,6-dimethyl-phenoxy; and wherein any phenyl-containing substituent of R₂ is optionally substituted with one to three substituents independently selected from the group consisting of C₁₋₆alkanyl, C₁₋₆alkanyloxy, fluoro, and hydroxy; or R₁ and R₂ taken together with the nitrogen to which they are attached form a pyrrolidinyl or piperidinyl ring wherein said pyrrolidinyl or piperidinyl is optionally substituted with a substituent selected from C₁₋₃alkanyl or hydroxy.

49. The compound according to claim 31 wherein R₃ is a substituent selected from the group consisting of benzo[1,3]dioxol-5-ylmethyl, carbamimidoyl, 1-H-imidazol-4-ylmethyl, phenyliminomethyl, 1-prop-2-ynyl, thioformyl, 2-hydroxyphenyl-methyl, hydroxy-ethyl, methoxy-ethyl, 2-methyl-allyl, 2-methyl-but-2-enyl, allyl, furan-3-ylmethyl, H, Me, methylthioethyl, phenethyl, pyridin-2-yl methyl, and thiophen-2-ylmethyl; and R₄ is one to two substituents independently selected from the group consisting of hydrogen, C₁₋₄alkanyl, C₁₋₄alkanyloxy, halogen, phenyl, furanyl, imidazolyl, indazolyl, indolyl, indolinyl, isoindolinyl, isoquinolinyl, isothiazolyl, isoxazolyl, oxazolyl, pyrazinyl, pyrazolyl, pyridazinyl, pyridinyl, pyrimidinyl, pyrrolyl, quinolinyl, tetrazolyl, thiazolyl, thienyl, and hydroxy.

50. The compound according to claim 31 wherein R₃ is a substituent selected from the group consisting of benzo[1,3]dioxol-5-ylmethyl, carbamimidoyl, 1-H-imidazol-4-yl methyl, phenyliminomethyl, 1-prop-2-ynyl, thioformyl, 2-hydroxyphenyl-methyl, hydroxyethyl, methoxyethyl, allyl, furan-3-yl methyl, H, Me, methylthioethyl, and phenethyl; R₄ is one to two substituents independently selected from the group consisting of hydrogen, methyl, methoxy, bromo, fluoro, α′- or β′-phenyl, α′- or β′-pyridinyl, α′- or β′-furanyl, and hydroxy.

51. The compound according to claim 31 wherein R₃ is a substituent selected from the group consisting of H, benzo[1,3]dioxol-5-ylmethyl, 1-H-imidazol-4-yl methyl, furan-3-ylmethyl, pyridin-2-ylmethyl, and phenyliminomethyl; and R₄ is a substituent independently selected from the group consisting of hydrogen, methyl, methoxy, bromo, fluoro, α′- or β′-phenyl, α′- or β′-pyridinyl, α′- or β′-furanyl, and hydroxy.

52. A compound of Formula (J): 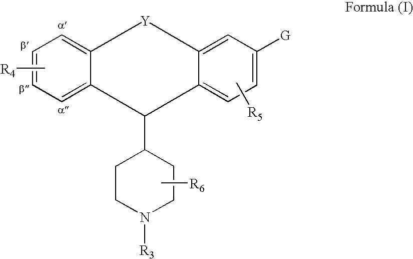
wherein:
G is —C(Z)N(R₁)R₂, phenyl, or a heterocycle selected from the group consisting of tetrazolyl, oxadiazolyl, and pyridinyl; wherein phenyl and the heterocycles of G are optionally substituted with one to three substituents independently selected from the group consisting of C₁₋₄alkanylcarbonylamino and oxo;
R₁ is selected from the group consisting of hydrogen and ethyl;
R₂ is selected from the group consisting of hydrogen and C₁₋₄alkanyl;
or R₁ and R₂ taken together with the nitrogen to which they are attached form a pyrrolidinyl ring optionally substituted with hydroxy;
R₃ is hydrogen, C₁₋₈alkanyl, C₂₋₈alkenyl, C₃₋₈cycloalkanyl, phenyl(C₁₋₈)alkanyl, or heteroaryl(C₁₋₈)alkanyl wherein the heteroaryl is imidazolyl, furanyl, pyridinyl, or thienyl;
R₄ is one substituent selected from the group consisting of hydrogen, methoxy, chloro, and hydroxy;
R₅ is hydrogen;
R₆ is hydrogen;
Y is O or S;
Z is O; and
enantiomers, diastereomers, tautomers, solvates, and pharmaceutically acceptable salts thereof.

53. A compound according to claim 52 wherein R₄ is hydrogen and Y is O.

54. A compound according to claim 52 wherein R₄ is α′-hydroxy and Y is O.

55. A compound according to claim 52 wherein R₄ is hydrogen and Y is S.

56. A compound according to claim 52 wherein R₄ is α′-hydroxy and Y is S.

57. A compound of Formula (I): 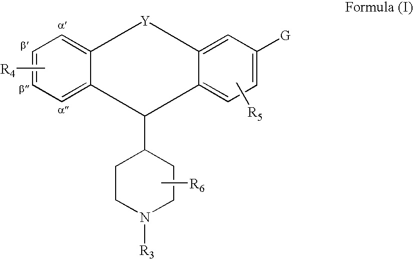
wherein:
G is N,N-diethylaminocarbonyl, 3-(N,N-diethylaminocarbonyl)-phenyl, 2-methylcarbonylaminophenyl, N—N-diethylamidino, pyridin-3-yl, 3-(S)-hydroxypyrrolidin-1-ylcarbonyl, N-ethylaminocarbonyl, 1H-tetrazol-4-yl, pyridine-4-yl, or 4H-[1,2,4]-oxadiazol-5-oxo-3-yl;
R₁ is selected from the group consisting of hydrogen and ethyl;
R₂ is hydrogen or ethyl;
or R₁ and R₂ taken together with the nitrogen to which they are attached form a pyrrolidinyl ring optionally substituted with hydroxy;
R₃ is hydrogen, methyl, ethyl, propenyl, cyclopropylmethyl, benzyl, phenethyl, or heteroaryl wherein the heteroaryl is furanyl, imidazolyl, pyridinyl, or thienyl;
R₄ is one substituent and is hydrogen or hydroxy;
R₅ is hydrogen;
R₆ is hydrogen;
Y is O or S;
Z is O; and
enantiomers, diastereomers, tautomers, solvates, and pharmaceutically acceptable salts thereof.

58. A compound according to claim 57 wherein R₄ is hydrogen and Y is O.

59. A compound according to claim 57 wherein R₄ is α′-hydroxy and Y is O.

60. A compound according to claim 57 wherein R₄ is hydrogen and Y is S.

61. A compound according to claim 57 wherein R₄ is α′-hydroxy and Y is S.

62. A compound that is:
N-{2-[5-Hydroxy-9-(1-pyridin-2-ylmethyl-piperidin-4-yl)-9H-xanthen-3-yl]-phenyl}-acetamide;
N-{2-[5-Hydroxy-9-(1-thiophen-2-ylmethyl-piperidin-4-yl)-9H-xanthen-3-yl]-phenyl}-acetamide;
6-Pyridin-4-yl-9-(1-pyridin-2-ylmethyl-piperidin-4-yl)-9H-thioxanthen-4-ol;
N-{2-[5-Hydroxy-9-(1-pyridin-2-ylmethyl-piperidin-4-yl)-9H-thioxanthen-3-yl]-phenyl}-acetamide;
N-[2-(5-Hydroxy-9-piperidin-4-yl-9H-thioxanthen-3-yl)-phenyl]-acetamide;
9-Piperidin-4-yl-6-pyridin-3-yl-9H-thioxanthen-4-ol;
6-Pyridin-3-yl-9-(1-pyridin-2-ylmethyl-piperidin-4-yl)-9H-thioxanthen-4-ol;
9-Piperidin-4-yl-6-pyridin-4-yl-9H-thioxanthen-4-ol;
5-Hydroxy-9-piperidin-4-yl-9H-xanthene-3-carboxylic acid diethylamide;
9-Piperidin-4-yl-6-(1H-tetrazol-5-yl)-9H-xanthen-4-ol;
9-Piperidin-4-yl-9H-xanthene-3-carboxylic acid diethylamide;
N,N-Diethyl-9-(1-furan-3-ylmethyl-piperidin-4-yl)-9H-xanthene-3-carboxamidine;
9-(1-Allyl-piperidin-4-yl)-6-pyridin-3-yl-9H-xanthen-4-ol;
N,N-Diethyl-3-(5-hydroxy-9-piperidin-4-yl-9H-xanthen-3-yl)-benzamide;
N-[2-(5-Hydroxy-9-piperidin-4-yl-9H-xanthen-3-yl)-phenyl]-acetamide;
N,N-Diethyl-9-piperidin-4-yl-9H-xanthene-3-carboxamidine;
4-(5-Methoxy-9-piperidin-4-yl-9H-thioxanthen-3-yl)-pyridine;
9-(1-Benzyl-piperidin-4-yl)-9H-xanthene-3-carboxylic acid diethylamide;
3-(5-Methoxy-9-piperidin-4-yl-9H-thioxanthen-3-yl)-pyridine;
9-(1-Benzyl-piperidin-4-yl)-6-pyridin-3-yl-9H-xanthen-4-ol;
6-Pyridin-3-yl-9-(1-thiophen-2-ylmethyl-piperidin-4-yl)-9H-xanthen-4-ol;
N-{2-[9-(1-Furan-3-ylmethyl-piperidin-4-yl)-9H-xanthen-3-yl]-phenyl}-acetamide;
9-[1-(1H-Imidazol-2-ylmethyl)-piperidin-4-yl]-9H-xanthene-3-carboxylic acid diethylamide;
N-[2-(5-Methoxy-9-piperidin-4-yl-9H-thioxanthen-3-yl)-phenyl]-acetamide;
N-{2-[9-(1-Allyl-piperidin-4-yl)-5-hydroxy-9H-thioxanthen-3-yl]-phenyl}-acetamide;
9-(1-Furan-3-ylmethyl-piperidin-4-yl)-9H-xanthene-3-carboxylic acid diethylamide;
3-(9-Piperidin-4-yl-9H-xanthen-3-yl)-pyridine;
5-Hydroxy-9-piperidin-4-yl-9H-xanthene-3-carbonitrile;
N-{2-[9-(1-Pyridin-2-ylmethyl-piperidin-4-yl)-9H-xanthen-3-yl]-phenyl}-acetamide;
[9-(1-Furan-3-ylmethyl-piperidin-4-yl)-9H-xanthen-3-yl]-(3-(S)-hydroxy-pyrrolidin-1-yl)-methanone;
5-Hydroxy-9-piperidin-4-yl-9H-xanthene-3-carboxylic acid ethylamide;
9-Piperidin-4-yl-6-pyridin-3-yl-9H-xanthen-4-ol;
5-Chloro-9-piperidin-4-yl-9H-xanthene-3-carboxylic acid diethylamide;
5-Methoxy-9-piperidin-4-yl-9H-xanthene-3-carboxylic acid diethylamide;
3-[9-(1-Furan-3-ylmethyl-piperidin-4-yl)-9H-xanthen-3-yl]-pyridine;
3-(5-Methoxy-9-piperidin-4-yl-9H-xanthen-3-yl)-pyridine;
(3-(S)-Hydroxy-pyrrolidin-1-yl)-(9-piperidin-4-yl-9H-xanthen-3-yl)methanone;
9-(1-Thiophen-2-ylmethyl-piperidin-4-yl)-9H-xanthene-3-carboxylic acid diethylamide;
1-Furan-3-ylmethyl-4-[3-(1H-tetrazol-5-yl)-9H-xanthen-9-yl]-piperidine;
4-(5-Methoxy-9-piperidin-4-yl-9H-xanthen-3-yl)-pyridine;
3-(9-Piperidin-4-yl-9H-xanthen-3-yl)-4H-[1,2,4]oxadiazol-5-one;
N-[2-(5-Methoxy-9-piperidin-4-yl-9H-xanthen-3-yl)-phenyl]-acetamide;
4-[3-(1H-Tetrazol-5-yl)-9H-xanthen-9-yl]-piperidine;
3-[9-(1-Benzyl-piperidin-4-yl)-9H-xanthen-3-yl]-pyridine;
4-[9-(1-Furan-3-ylmethyl-piperidin-4-yl)-9H-xanthen-3-yl]-pyridine;
N-[2-(9-Piperidin-4-yl-9H-xanthen-3-yl)-phenyl]-acetamide;
9-(1-Cyclopropylmethyl-piperidin-4-yl)-9H-xanthene-3-carboxylic acid diethylamide;
N-{2-[9-(1-Allyl-piperidin-4-yl)-9H-xanthen-3-yl]-phenyl}-acetamide;
9-(1-Methyl-piperidin-4-yl)-9H-xanthene-3-carboxylic acid diethylamide;
4-[9-(1-Pyridin-2-ylmethyl-piperidin-4-yl)-9H-xanthen-3-yl]-pyridine;
9-(1-Pyridin-2-ylmethyl-piperidin-4-yl)-9H-xanthene-3-carboxylic acid diethylamide;
3-[9-(1-Phenethyl-piperidin-4-yl)-9H-xanthen-3-yl]-pyridine;
9-(1-Phenethyl-piperidin-4-yl)-9H-xanthene-3-carboxylic acid diethylamide;
4-(9-Piperidin-4-yl-9H-xanthen-3-yl)-pyridine;
N-{2-[9-(1-Phenethyl-piperidin-4-yl)-9H-xanthen-3-yl]-phenyl}-acetamide;
(3-(S)-Hydroxy-pyrrolidin-1-yl)-[9-(1-phenethyl-piperidin-4-yl)-9H-xanthen-3-yl]-methanone;
[9-(1-Allyl-piperidin-4-yl)-9H-xanthen-3-yl]-(3-(S)-hydroxy-pyrrolidin-1-yl)methanone;
3-{9-[1-(1H-Imidazol-2-ylmethyl)-piperidin-4-yl]-9H-xanthen-3-yl}-pyridine;
3-[9-(1-Thiophen-2-ylmethyl-piperidin-4-yl)-9H-xanthen-3-yl]-pyridine;
1-Phenethyl-4-[3-(1H-tetrazol-5-yl)-9H-xanthen-9-yl]-piperidine;
3-{9-[1-(1H-Imidazol-2-ylmethyl)-piperidin-4-yl]-9H-xanthen-3-yl}-4H-[1,2,4]oxadiazol-5-one;
9-Piperidin-4-yl-6-pyridin-4-yl-9H-xanthen-4-ol;
3-[9-(1-Furan-3-ylmethyl-piperidin-4-yl)-9H-xanthen-3-yl]-4H-[1,2,4]oxadiazol-5-one;
N,N-Diethyl-9-(1-phenethyl-piperidin-4-yl)-9H-xanthene-3-carboxamidine;
N-{2-[9-(1-Furan-3-ylmethyl-piperidin-4-yl)-5-hydroxy-9H-thioxanthen-3-yl]-phenyl}-acetamide;
N,N-Diethyl-5-hydroxy-9-piperidin-4-yl-9H-xanthene-3-carboxamidine; or
5-Hydroxy-9-(1-pyridin-2-ylmethyl-piperidin-4-yl)-9H-xanthene-3-carbonitrile.

63. A compound that is:
N-{2-[5-Hydroxy-9-(1-pyridin-2-ylmethyl-piperidin-4-yl)-9H-xanthen-3-yl]-phenyl}-acetamide;
N-{2-[5-Hydroxy-9-(1-thiophen-2-ylmethyl-piperidin-4-yl)-9H-xanthen-3-yl]-phenyl}-acetamide;
6-Pyridin-4-yl-9-(1-pyridin-2-ylmethyl-piperidin-4-yl)-9H-thioxanthen-4-ol;
N-{2-[5-Hydroxy-9-(1-pyridin-2-ylmethyl-piperidin-4-yl)-9H-thioxanthen-3-yl]-phenyl}-acetamide;
N-[2-(5-Hydroxy-9-piperidin-4-yl-9H-thioxanthen-3-yl)-phenyl]-acetamide;
9-Piperidin-4-yl-6-pyridin-3-yl-9H-thioxanthen-4-ol;
6-Pyridin-3-yl-9-(1-pyridin-2-ylmethyl-piperidin-4-yl)-9H-thioxanthen-4-ol;
9-Piperidin-4-yl-6-pyridin-4-yl-9H-thioxanthen-4-ol;
5-Hydroxy-9-piperidin-4-yl-9H-xanthene-3-carboxylic acid diethylamide; or
9-Piperidin-4-yl-6-(1H-tetrazol-5-yl)-9H-xanthen-4-ol.

64. A composition comprising the dextrorotatory enantiomer of a compound of claim 1 wherein said composition is substantially free from the levorotatory isomer of said compound.

65. A composition comprising the levororotatory enantiomer of a compound of claim 1 wherein said composition is substantially free from the dextrorotatory isomer of said compound.

66. A pharmaceutical composition comprising a compound, salt or solvate according to claim 1 admixed with a pharmaceutically acceptable carrier, excipient or diluent.

67. A veterinary composition comprising a compound, salt or solvate according to claim 1 admixed with a veterinarily acceptable carrier, excipient or diluent.

68. A pharmaceutical composition comprising a compound, salt or solvate according to claim 42 admixed with a pharmaceutically acceptable carrier, excipient or diluent.

69. A veterinary composition comprising a compound, salt or solvate according to claim 42 admixed with a veterinarily acceptable carrier, excipient or diluent.

70. A pharmaceutical composition comprising a compound, salt or solvate according to claim 43 admixed with a pharmaceutically acceptable carrier, excipient or diluent.

71. A veterinary composition comprising a compound, salt or solvate according to claim 43 admixed with a veterinarily acceptable carrier, excipient or diluent.

72. A pharmaceutical composition comprising a compound, salt or solvate according to claim 47 admixed with a pharmaceutically acceptable carrier, excipient or diluent.

73. A veterinary composition comprising a compound, salt or solvate according to claim 47 admixed with a veterinarily acceptable carrier, excipient or diluent.

74. A pharmaceutical composition comprising a compound, salt or solvate according to claim 52 admixed with a pharmaceutically acceptable carrier, excipient or diluent.

75. A veterinary composition comprising a compound, salt or solvate according to claim 52 admixed with a veterinarily acceptable carrier, excipient or diluent.

76. A pharmaceutical composition comprising a compound, salt or solvate according to claim 57 admixed with a pharmaceutically acceptable carrier, excipient or diluent.

77. A veterinary composition comprising a compound, salt or solvate according to claim 57 admixed with a veterinarily acceptable carrier, excipient or diluent.

78. A pharmaceutical composition comprising a compound, salt or solvate according to claim 62 admixed with a pharmaceutically acceptable carrier, excipient or diluent.

79. A veterinary composition comprising a compound, salt or solvate according to claim 62 admixed with a veterinarily acceptable carrier, excipient or diluent.

80. A pharmaceutical composition comprising a compound, salt or solvate according to claim 63 admixed with a pharmaceutically acceptable carrier, excipient or diluent.

81. A veterinary composition comprising a compound, salt or solvate according to claim 63 admixed with a veterinarily acceptable carrier, excipient or diluent.
